SEQUENCE LISTING (6 sequences)

SEQ ID NO: 1 - protein, 5 aa, Murinae gen. sp.
SYDMS

SEQ ID NO: 2 - protein, 7 aa, Murinae gen. sp.
KVSSGGG

SEQ ID NO: 3 - protein, 8 aa, Murinae gen. sp.
HNYGSFAY

SEQ ID NO: 4 - protein, 11 aa, Murinae gen. sp.
QASQSISNHLH

SEQ ID NO: 5 - protein, 7 aa, Murinae gen. sp.
YRSQSIS

SEQ ID NO: 6 - protein, 9 aa, Murinae gen. sp.
QQSGSWPHT

(19) United States
(12) Patent Application Publication
(10) Pub. No.: US 2009/0053238 A1
(43) Pub. Date: Feb. 26, 2009

(54) Stabilized High Concentration Anti-Integrin alphavbeta3 Antibody Formulations

(75) Inventor: Christian B. ALLAN, Brookeville, MD (US)

Correspondence Address: MEDIMMUNE, LLC;Jonathan Klein-Evans, ONE MEDIMMUNE WAY, GAITHERSBURG, MD, 20878, US

(73) Assignee: MEDIMMUNE, INC., Gaithersburg, MD (US)

(21) Appl. No.: 11/462,277

(22) Filed: Aug. 3, 2006

Related U.S. Application Data
(63) Continuation of application No. 10/769,712, filed on Jan. 30, 2004, now abandoned.
(60) Provisional application No. 60/443,777, filed on Jan. 30, 2003.

(51) Int. Cl. A61K 39/395

(52) U.S. Cl. 424/172.1

(57) ABSTRACT

The present invention provides liquid formulations of antibodies or antibody fragments that immunospecifically bind to integrin α_{V}β₃, which formulations exhibit stability, low to undetectable levels of aggregation, and very little to no loss of the biological activities of the antibodies or antibody fragments, even during long periods of storage. In particular, the present invention provides liquid formulations of antibodies or fragments thereof that immunospecifically bind to integrin α_{V}β₃, which formulations are substantially free of surfactant, inorganic salts, and/or other common excipients. Furthermore, the invention provides methods of preventing, treating or ameliorating an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer utilizing the liquid formulations of the present invention.

This application is entitled to and claims priority benefits to U.S. Provisional Application Ser. No. 60/443,777, filed Jan. 30, 2003, which is incorporated herein by reference in its entirety.

### 1. INTRODUCTION

The present invention relates to high concentration liquid formulations of antibodies or fragments thereof that immunospecifically bind to integrin α_{V}β₃ which formulations exhibit stability, low to undetectable levels of antibody fragmentation, low to undetectable levels of aggregation, and very little to no loss of the biological activities of the antibodies or antibody fragments, even during long periods of storage. In particular, the present invention relates to liquid formulations of antibodies or fragments thereof that immunospecifically bind to integrin α_{V}β₃, which formulations are substantially free of surfactant, inorganic salts or both. The present invention also relates to methods of preventing, treating, managing or ameliorating inflammatory diseases, autoimmune diseases, disorders associated with aberrant expression and/or activity of integrin α_{V}β₃, disorders associated with abnormal bone metabolism, disorders associated with aberrant angiogenesis and cancers, utilizing high concentration liquid formulations of antibodies or fragments thereof that immunospecifically bind to integrin α_{V}β₃.

### 2. BACKGROUND OF THE INVENTION

Currently, many antibodies are provided as lyophilized formulations. Lyophilized formulations of antibodies have a number of limitations, including a prolonged process for lyophilization and resulting high cost for manufacturing. In addition, a lyophilized formulation has to be reconstituted aseptically and accurately by healthcare practitioners prior to administering to patients. The reconstitution step itself requires certain specific procedures: (1) a sterile diluent (i.e., water of intravenous administration and 5% dextrose in water for intramuscular administration) is added to the vial containing lyophilized antibody, slowly and aseptically, and the vial must be swirled very gently for 30 seconds to avoid foaming; (2) the reconstituted antibody may need to stand at room temperature for a minimum of 20 minutes until the solution clarifies; and (3) the reconstituted preparation must be administered within six (6) hours after the reconstitution. Such reconstitution procedure is cumbersome and the time limitation after the reconstitution can cause a great inconvenience in administering the formulation to patients, leading to significant waste, if not reconstituted properly or if the reconstituted dose is not used within six (6) hours and must be discarded.

Thus, a need exists for liquid formulations of antibodies, in particular, anti-integrin α_{V}β₃ antibodies, at a concentration comparable to or higher than the reconstituted lyophilized formulations so that there is no need to reconstitute the formulation prior to administration. This allows healthcare practitioners much quicker and easier administration of antibodies to a patient.

Prior liquid antibody preparations have short shelf lives and may lose biological activity of the antibodies resulting from chemical and physical instabilities during the storage. Chemical instability may be caused by deamidation, racemization, hydrolysis, oxidation, beta elimination or disulfide exchange, and physical instability may be caused by antibody denaturation, aggregation, precipitation or adsorption. Among those, aggregation, deamidation and oxidation are known to be the most common causes of the antibody degradation (Wang et al., 1988, J. of Parenteral Science & Technology 42(Suppl):S4-S26; Cleland et al., 1993, Critical Reviews in Therapeutic Drug Carrier Systems 10(4):307-377). Thus, there is a need for a stable liquid formulation of antibodies, in particular, stable liquid anti-integrin α_{V}β₃ antibodies.

### 3. SUMMARY OF INVENTION

The present invention is based, in part, on the development of high concentration liquid formulations of antibodies or fragments thereof that immunospecifically bind to integrin α_{V}β₃, which formulations exhibit, in the absence of surfactant and/or inorganic salt, stability, low to undetectable levels of antibody fragmentation and/or aggregation, and very little to no loss of the biological activities of the antibodies or antibody fragments during manufacture, preparation, transportation, and storage. The liquid formulations of the present invention facilitate the administration of antibodies or fragments thereof that immunospecifically bind to integrin α_{V}β₃ for the prevention, treatment, management and amelioration of one or more symptoms associated with inflammatory diseases and autoimmune diseases, including, but not limited to: rheumatoid arthritis, scapulohumeral periarthritis, multiple sclerosis, psoriasis, myasthenia gravis, vasculitis, pemphigus vulgaris, ulcerative colitis, Crohn's disease, Hashimoto's thyroiditis, systemic lupus erythematosus, scleroderma, polymyositis, osteolysis, ankylosing spondylitis, psoriatic arthritis and Sjogren's syndrome. The liquid formulations of the present invention also facilitate the administration of antibodies or fragments thereof that immunospecifically bind to integrin α_{V}β₃ for the prevention, treatment, management and amelioration of one or more symptoms associated with cancer including, but not limited to: glioblastoma, bone cancer, breast cancer and colon cancer. The liquid formulations of the present invention also facilitate the administration of antibodies or fragments thereof that immunospecifically bind to integrin α_{V}β₃ for the prevention, treatment, management and amelioration of one or more symptoms associated with abnormal bone metabolism including, but not limited to: degenerative joint disease (osteoarthritis), gouty arthritis, chondrocalcinosis, and tumor and tumor-like lesions of bone (non-limiting examples are primary tumors of bone, secondary or metastatic tumors of bone, neurogenic arthropathy, and arthritis in sarcoidosis). In particular, the liquid formulations of the present invention enable a healthcare professional to quickly administer a sterile dosage of an antibody or an antibody fragment that immunospecifically binds to integrin α_{V}β₃ without having to accurately and sterilely reconstitute the antibody or antibody fragment prior to administration.

The present invention provides liquid formulations substantially free of surfactant and/or inorganic salts, said formulations comprising histidine and a concentration of 50 mg/ml or higher of antibodies or fragments thereof that immunospecifically bind to integrin α_{V}β₃. The present invention also provides liquid formulations substantially free of surfactant and/or inorganic salt, said formulations having a pH ranging from about 5.0 to about 7.0, preferably about pH 6.0, and comprising histidine, and a concentration of 50 mg/ml or higher of antibodies or antibody fragments that immunospecifically bind to integrin α_{V}β₃. The liquid formulations of the present invention may further comprise one or more excipients such as a saccharide, an amino acid (e.g., arginine, lysine, and methionine) and a polyol. In a preferred embodiment, a liquid formulation of the present invention comprises histidine and a concentration of 95 mg/ml or higher of antibodies or antibody fragments that immunospecifically bind to integrin α_{V}β₃, and said formulation is substantially free of surfactant and salt.

The present invention encompasses stable liquid formulations of Vitaxin® (see, e.g., Wu et al., 1998, PNAS USA 95(11):6037-6042) which exhibit low to undetectable levels of antibody aggregation and/or fragmentation with very little to no loss of the biological activities of Vitaxin® during manufacture, preparation, transportation, and long periods of storage. The present invention also encompasses stable liquid formulations of antibodies that immunospecifically bind to integrin α_{V}β₃₃ and have increased in vivo half-lives relative to known antibodies such as, e.g., Vitaxin®, said formulations exhibiting low to undetectable levels of antibody aggregation and/or fragmentation, and very little to no loss of the biological activities of the antibodies or antibody fragments. The present invention also encompasses stable liquid formulations of antibodies that immunospecifically bind to integrin α_{V}β₃, said antibodies or antibody fragments comprising a variable heavy (VH) and/or variable light (VL) domain having the amino acid sequence of the VH and/or VL domain of LM609 or VITAXIN®, said formulations exhibiting low to undetectable levels of antibody aggregation and/or fragmentation, and very little to no loss of the biological activities of the antibodies or antibody fragments. The present invention further encompasses stable liquid formulations of antibodies or antibody fragments that immunospecifically bind to integrin α_{V}β₃, said antibodies or antibody fragments comprising one or more VH complementarity determining regions (CDRs) and/or one or more VL CDRs having the amino acid sequence of one or more VH CDRs and/or VL CDRs listed in Table 1, said formulations exhibiting low to undetectable levels of antibody aggregation and/or fragmentation, and very little to no loss of the biological activities of the antibodies or antibody fragments.

TABLE 1

CDR Sequences Of LM609

| | | |
|---|---|---|
| CDR | Sequence | SEQ ID NO: |

| | | |
|---|---|---|
| VH1 | SYDMS | 1 |
| VH2 | KVSSGGG | 2 |
| VH3 | HNYGSFAY | 3 |
| VL1 | QASQSISNHLH | 4 |
| VL2 | YRSQSIS | 5 |
| VL3 | QQSGSWPHT | 6 |

The present invention encompasses liquid formulations of antibodies or antibody fragments that immunospecifically bind to integrin α_{V}β₃, said formulations having stability at 38-42° C. as assessed by high performance size exclusion chromatography (HPSEC). The liquid formulations of the present invention exhibit stability, as assessed by HPSEC, at temperature ranges of 38-42° C. for at least 15 days but no more than 25 days; at temperature ranges of 20-24° C. for at least 6 months but not more than 1.5 years; and at temperature ranges of 2-8° C. (especially at 4° C.) for at least 1.5 years, at least 2 years, at least 2.5 years, or at least 3 years. The present invention also encompasses liquid formulations of antibodies or fragments thereof that immunospecifically bind to integrin α_{V}β₃ said formulations having low to undetectable levels of antibody aggregation as measured by HPSEC. In a preferred embodiment, the liquid formulations of the present invention exhibit stability at 38-42° C. for at least 15 days and exhibit low to undetectable levels of antibody aggregation as measured by HPSEC and, further, exhibit very little to no loss of the biological activity of the antibodies or antibody fragments of the formulation compared to the reference antibodies as measured by antibody binding assays such as, e.g., ELISAs.

The present invention provides methods for preparing liquid formulations of an antibody or antibody fragment that immunospecifically binds to integrin α_{V}β₃, said methods comprising concentrating a fraction containing the purified antibody to a final antibody concentration ranging from about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 150 mg/ml, about 175 mg/ml, or about 200 mg/ml using a semipermeable membrane with an appropriate molecular weight (MW) cutoff (e.g., a 30 kD cutoff for whole antibody molecules and F(ab′)₂ fragments; and a 10 kD cutoff for antibody fragments such as Fab fragments) and diafiltering the concentrated antibody fraction into the formulation buffer using the same membrane. The formulation buffer of the present invention comprises histidine at a concentration ranging from about 1 mM to about 100 mM, preferably from about 5 mM to about 50 mM, more preferably about 10 mM to about 25 mM. The pH of the formulation may range from about 5.0 to about 7.0, preferably 5.5 to about 6.5, more preferably about 5.8 to about 6.2, and most preferably about 6.0. To obtain an appropriate pH for a particular antibody, it is preferable that histidine (and glycine, if added) is first dissolved in water to obtain a buffer solution with higher pH than the desired pH and then the pH is brought down to the desired level by the addition of HCl. This way, the formation of inorganic salts (e.g., the formation of NaCl when, e.g., histidine hydrochloride is used as the source of histidine and the pH is raised to the desired level by the addition of NaOH) can be avoided.

The liquid formulations of the present invention may be sterilized by sterile filtration using a 0.2μ filter. Sterilized liquid formulations of the present invention may be administered to a subject to prevent, treat, manage or ameliorate inflammatory diseases, autoimmune diseases, disorders associated with abnormal bone metabolism, disorders associated with abnormal angiogenesis, disorders associated with aberrant expression and/or activity of integrin α_{V}β₃, cancer, or one or more symptoms thereof. The liquid formulations of the present invention may be administered in combination with other therapies (e.g., prophylactic or therapeutic agents other than antibodies that immunospecifically bind to integrin α_{V}β₃, such as anti-inflammatory agents, anti-cancer agents, bone metabolism regulating agents, immunomodulatory agents and anti-angiogenic agents).

The present invention also provides kits comprising the liquid formulations of antibodies or antibody fragments that immunospecifically bind to integrin α_{V}β₃ for use by, e.g., a healthcare professional. The present invention further provides methods of preventing, treating, managing or ameliorating inflammatory diseases, autoimmune diseases, disorders associated with abnormal bone metabolism, disorders associated with abnormal angiogenesis, disorders associated with aberrant expression and/or activity of integrin α_{V}β₃, and cancer by administering the liquid formulations of the present invention. The liquid formulations of the invention can be administered parenterally (e.g., intradermally, intramuscularly, intraperitoneally, intravenously and subcutaneously) or orally to a subject to prevent, treat, manage or ameliorate an inflammatory disease, an autoimmune disease, a disorder associated with abnormal bone metabolism, a disorder associated with abnormal angiogenesis, a disorder associated with aberrant expression and/or activity of integrin α_{V}β₃ or cancer.

The liquid formulations of the present invention can also be used to diagnose, detect or monitor disorders associated with abnormal expression of integrin α_{V}β₃, inflammatory diseases, autoimmune diseases, disorders associated with abnormal bone metabolism, disorders associated with abnormal angiogenesis and cancer.

### 3.1. TERMINOLOGY

All liquid formulations of antibodies and/or antibody fragments that immunospecifically bind to integrin α_{V}β₃ described above are herein collectively referred to as “liquid formulations of the invention,” “antibody liquid formulations of the invention,” “antibody formulations of the invention,” “liquid formulations of antibodies or fragments thereof that immunospecifically bind to integrin α_{V}β₃,” or “liquid formulations of anti-integrin α_{V}β₃ antibodies.”

As used herein the term “aberrant” refers to a deviation from the norm, e.g., the average healthy subject and/or a population of average healthy subjects. The term “aberrant expression,” as used herein, refers to abnormal expression of a gene product (e.g., RNA, protein, polypeptide, or peptide) by a cell or subject relative to a normal, healthy cell or subject and/or population of normal, healthy cells or subjects. Such aberrant expression may be the result of the amplification of the gene. In a specific embodiment, the term “aberrant expression” refers to abnormal expression of integrin αvβ3 by a cell or subject relative to the expression of the gene product by a normal, healthy cell or subject and/or population of normal, healthy cells or subjects and encompasses the expression of an integrin αvβ3 gene product at an unusual location within the cell or subject, the expression of an integrin αvβ3 gene product at an altered level in the cell or subject, the expression of a mutated integrin αvβ3 gene product, or a combination thereof. In another embodiment, the term “aberrant expression” refers to the overexpression of an integrin αvβ3 gene product and/or an integrin αvβ3 gene product by a cell or subject relative to the expression of an integrin αvβ3 gene product by a normal, healthy cell or subject and/or population of normal, healthy cells or subjects. In accordance with this embodiment, the overexpression may be the result of gene amplification. The term “aberrant activity,” as used herein, refers to an altered level of a gene product, the increase of an activity by a gene product, or the loss of an activity of a gene product in a cell or subject relative to a normal, healthy cell or subject and/or population of normal, healthy cells or subjects. In a specific embodiment, the term “aberrant activity” refers to an integrin αvβ3 activity that deviates from that normally found in a healthy cell or subject and/or population of healthy cells or subjects. In another embodiment, the term “aberrant activity” refers to an increase in an integrin αvβ3 activity relative to that normally found in a healthy cell or subject and/or population of healthy cells or subjects. Examples of integrin αvβ3 activities include, but are not limited to, being a receptor for fibrinogen, fibronectin, von Willebrand's factor, Vitronectin, Tsp (Thrombospondin), osteopontin and Bsp1 (bone sialoprotein 1), mediating platelet aggregation and endothelial cell adhesion to ECM proteins, and promoting angiogenesis. In certain embodiments, an integrin α_{V}β₃ exhibits aberrant activity, such as but not limited to, forming stronger bond with its ligand. Examples of integrin α_{V}β₃ ligands include, but are not limited to, vitronectin, osteopontin, bone sialoprotein, echistatin, RGD-containing peptides, RGD mimetics and blocking antibodies. (See e.g., Dresner-Pollak et al., J. Cell Biochem. 56(3):323-30; Duong et al., Front. Biosci. 1(3):d757-68).

As used herein, the term “about” in the context of a given numerate value or range refers to a value or range that is within 20%, preferably within 10%, and more preferably within 5% of the given value or range.

As used herein, the term “angiogenesis” refers to the growth of new blood vessels. Accordingly, “abnormal angiogenesis” or “aberrant angiogenesis” refers to altered (e.g., increased or decreased) activity of angiogenesis, i.e., any angiogenesis that deviates from the normal process of angiogenesis, such as but not limited to, increased angiogenesis activity in a body, and angiogenesis at an abnormal location of the body. A disease or disorder may be completely caused by abnormal angiogenesis or may be exacerbated by abnormal angiogenesis. Abnormal angiogenesis-related diseases or disorders due to excessive angiogenesis may include, but are not limited to, cancer, tumors, rheumatoid arthritis, psoriasis, rosacea and metastasis of cancerous cells. Abnormal angiogenesis-related disorders due to insufficient angiogenesis may include, but are not limited to, coronary artery disease, stroke, ulcers and delayed wound healing. In some embodiments, “abnormal angiogenesis” refers to increased or excessive activity of angiogenesis.

As used herein, the term “analog” in the context of proteinaceous agent (e.g., proteins, polypeptides, peptides, and antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possess a similar or identical function as a first organic or inorganic molecule and is structurally similar to the first organic or inorganic molecule.

The term “antibody fragment” as used herein refers to a fragment of an antibody that immunospecifically binds to integrin α_{V}β₃. Antibody fragments may be generated by any technique known to one of skill in the art. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the complete light chain, and the variable region, the CH1 region and the hinge region of the heavy chain. Antibody fragments can be also produced by recombinant DNA technologies. Antibody fragments may be one or more complementarity determining regions (CDRs) of antibodies, or one or more antigen-binding fragments of an antibody.

The terms “antibody” and “antibodies” as used herein refer to monoclonal antibodies, bispecific antibodies, multispecific antibodies, human antibodies, humanized antibodies, chimeric antibodies, camelised antibodies, single-chain Fvs (scFv), single chain antibodies, disulfide-linked Fvs (sdFv), and anti-idiotypic (anti-Id) antibodies (including, e.g., anti-Id antibodies to antibodies of the invention). In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

The term “antibodies or antibody fragments that immunospecifically bind to integrin α_{V}β₃” and analogous terms as used herein refer to antibodies or antibody fragments that specifically bind to an integrin α_{V}β₃ polypeptide or a fragment of an integrin α_{V}β₃ polypeptide and do not specifically bind to other polypeptides. Preferably, antibodies or antibody fragments that immunospecifically bind to an integrin α_{V}β₃ polypeptide or fragment thereof do not cross-react with other antigens. Antibodies or antibody fragments that immunospecifically bind to an integrin α_{V}β₃ polypeptide can be identified, for example, by immunoassays or other techniques known to those of skill in the art. See, e.g., Paul ed., 1989, Fundamental Immunology, 2^{nd} ed., Raven Press, New York at pages 332-336 for a discussion regarding antibody specificity. Preferably antibodies or antibody fragments that immunospecifically bind to an integrin α_{V}β₃ polypeptide or fragment thereof only antagonize integrin α_{V}β₃ and do not significantly antagonize other activities, such as activities of an integrin comprising αᵥ but not β3 (e.g., integrin αᵥβ1, which is a receptor for fibronectin).

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e, by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

The terms “disorder” and “disease” are used herein interchangeably to refer to a condition in a subject. In particular, the term “autoimmune disease” is used interchangeably with the term “autoimmune disorder” to refer to a condition in a subject characterized by cellular, tissue and/or organ injury caused by an immunologic reaction of the subject to its own cells, tissues and/or organs. The term “inflammatory disease” is used interchangeably with the term “inflammatory disorder” to refer to a condition in a subject characterized by inflammation, preferably chronic inflammation. Autoimmune disorders may or may not be associated with inflammation. Moreover, inflammation may or may not be caused by an autoimmune disorder. Certain conditions may be characterized as more than one disorder, for example, certain disorders may be characterized as both autoimmune and inflammatory disorders.

As used herein, the term “effective amount” in the context of therapy refers to the amount of a therapy (e.g., a prophylactic or therapeutic agent) which is sufficient to reduce and/or ameliorate the severity and/or duration of a disease or disorder (e.g., an inflammatory disease, an autoimmune disease, a disorder associated with abnormal bone metabolism, a disorder associated with abnormal angiogenesis, a disorder associated with aberrant expression and/or activity of integrin αvβ3, or cancer) or a symptom thereof, prevent the advancement of said disorder or disease, cause regression of said disorder or disease, prevent the recurrence, development, or onset of one or more symptoms associated with said disorder or disease, or enhance or improve the prophylactic or therapeutic effect(s) of another therapy (e.g., prophylactic or therapeutic agent).

As used herein, the term “effective amount” in the context of detecting the expression of integrin αvβ3 refers to the amount of antibodies, antibody fragments, or a formulation comprising antibodies or antibody fragments which is sufficient to detect integrin αvβ3 expression.

The term “epitope” as used herein refers to portions of an integrin α_{V}β₃ polypeptide having antigenic or immunogenic activity in an animal, preferably in a mammal, and most preferably in a human. An epitope having immunogenic activity is a portion of an integrin α_{V}β₃ polypeptide that elicits an antibody response in an animal. An epitopes having antigenic activity is a portion of an integrin α_{V}β₃ polypeptide to which an antibody immunospecifically binds as determined by any method well known in the art, for example, by the immunoassays described herein (see Section 5.4.3 infra). Antigenic epitopes need not necessarily be immunogenic.

The term “excipient” as used herein refers to an inert substance which is commonly used as a diluent, vehicle, preservative, binder or stabilizing agent for drugs which imparts a beneficial physical property to a formulation, such as increased protein stability, increased protein solubility, and decreased viscosity. Examples of excipients include, but are not limited to, proteins (e.g., serum albumin), amino acids (e.g., aspartic acid, glutamic acid, lysine, arginine, glycine and histidine), surfactants (e.g., SDS, polysorbate and nonionic surfactant), saccharides (e.g., glucose, sucrose, maltose and trehalose), polyols (e.g., mannitol and sorbitol), fatty acids and phospholipids (e.g., alkyl sulfonates and caprylate). For additional information regarding excipients, see Remington's Pharmaceutical Sciences (by Joseph P. Remington, 18^{th} ed., Mack Publishing Co., Easton, Pa.), which is incorporated herein in its entirety.

The term “fragment” as used herein refers to a peptide, polypeptide, or protein (including an antibody) comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide or protein. In a specific embodiment, a fragment of a protein or polypeptide retains at least one function of the protein or polypeptide. In another embodiment, a fragment of a protein or polypeptide retains at least two, three or four functions of the protein or polypeptide. Preferably a fragment of an antibody that immunospecifically binds to integrin α_{V}β₃ retains the ability to bind to integrin α_{V}β₃. A “functional fragment” is a fragment that retains at least one function of the protein or polypeptide.

The term “fusion protein” as used refers to a polypeptide or protein that comprises an amino acid sequence of a first protein, polypeptide or functional fragment, analog or derivative thereof, and an amino acid sequence of a heterologous protein or polypeptide (i.e., a second protein, polypeptide or functional fragment, analog or derivative thereof different than the first protein, polypeptide or functional fragment, analog or derivative thereof, or a second protein, polypeptide or functional fragment, analog or derivative thereof not naturally found conjugated to the first protein, polypeptide or functional fragment, analog, or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent.

The terms “high concentration” and “concentrated antibody” as used herein refer to a concentration of 50 mg/ml or higher, preferably 95 mg/ml or higher of an antibody or fragment thereof that immunospecifically binds to integrin α_{V}β₃, in an antibody formulation.

The term “host cell” as used herein includes a subject cell transfected or transformed with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “hybridizes under stringent conditions” as used herein describes conditions for hybridization and washing under which nucleotide sequences at least 30% (preferably at least 35%, at least 40%, at least 45%, at least 50%, at least 55%, at least 60%, at least 65%, at least 70%, at least 75%, at least 80%, at least 85%, at least 90%, or at least 95%) identical to each other typically remain hybridized to each other. Such stringent conditions are known to those skilled in the art and can be found in Current Protocols in Molecular Biology, John Wiley & Sons, N.Y. (1989), 6.3.1-6.3.6. Generally, stringent conditions are selected to be about 5 to 10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

In one, non-limiting example stringent hybridization conditions are hybridization at 6× sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

The term “immunomodulatory agent” and variations thereof including, but not limited to, immunomodulatory agents, as used herein refer to an agent that modulates a host's immune system. In certain embodiments, an immunomodulatory agent is an immunosuppressant agent. In certain other embodiments, an immunomodulatory agent is an immunostimulatory agent. In accordance with the invention, an immunomodulatory agent used in the combination therapies of the invention does not include an anti-integrin αᵥβ₃ antibody or fragment thereof. Immunomodulatory agents include, but are not limited to, small molecules, peptides, polypeptides, proteins, fusion proteins, antibodies, inorganic molecules, mimetic agents, and organic molecules.

The term “in combination” as used herein refers to the use of more than one therapies (e.g., prophylactic and/or therapeutic agents). The use of the term “in combination” does not restrict the order in which therapies (e.g., prophylactic and/or therapeutic agents) are administered to a subject with a disease or disorder (e.g., an inflammatory disease, an autoimmune disease, a disorder associated with abnormal bone metabolism, a disorder associated with abnormal angiogenesis, a disorder associated with aberrant expression and/or activity of integrin αvβ3, or cancer). A first therapy (e.g., a prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a disease or disorder (e.g., an inflammatory disease, an autoimmune disease, a disorder associated with abnormal bone metabolism, a disorder associated with abnormal angiogenesis, a disorder associated with aberrant expression and/or activity of integrin αvβ3, or cancer).

The term “inorganic salt” as used herein refers to any compounds containing no carbon that result from replacement of part or all of the acid hydrogen or an acid by a metal or a group acting like a metal and are often used as tonicity adjusting compounds in pharmaceutical compositions and preparations of biological materials. The most common inorganic salt are NaCl, KCl, NaH₂PO₄, etc.

The term “α_{V}β₃” or “integrin α_{V}β₃” refers to the heterodimer of the integrin subunit α_{V} and the integrin subunit β₃ and includes analogs, derivatives or fragments of the subunits of the heterodimer, and fusion proteins comprising the heterodimer integrin α_{V}β₃ analogs, derivatives or a fragments of the subunits of the heterodimer. The integrin α_{V}β₃ may be from any species. The nucleotide and/or amino acid sequences of integrin α_{V}β₃ can be found in the literature or public databases, or the nucleotide and/or amino acid sequences can be determined using cloning and sequencing techniques known to one of skill in the art. For example, the nucleotide sequence of human integrin α_{V}β₃ can be found in the GenBank database (see, e.g., Accession No. NM_{—}002210 for α_{V}, and Accession No. L28832 for β₃). The amino acid sequence of human α_{V}β₃ can be found in the GenBank database (see, e.g., Accession No. AAA 61631 for α_{V}, and Accession No. S44360 for β₃). In a preferred embodiment, an integrin α_{V}β₃ is human integrin α_{V}β₃, an analog, derivative or a fragment thereof. Antibodies and antibody fragments that immunospecifically bind to an integrin αvβ3 that is a fusion protein bind to the portion of the fusion protein comprising the heterodimer integrin αvβ3, an analog, derivative or a fragment thereof.

As used herein, the term “isolated” or “purified” in the context of a proteinaceous agent (e.g., a peptide, polypeptide, fusion protein, or antibody) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, 5%, 1%, 0.5%, or 0.1% (by dry weight) of heterologous protein, polypeptide, peptide, or antibody (also referred to as a “contaminating protein”). When the proteinaceous agent is recombinantly produced, it is also preferably substantially free of culture medium, i.e., culture medium represents less than about 20%, 10%, 5%, 1%, 0.5%, or 0.1% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5%, 1%, 0.5%, or 0.1% (by dry weight) of chemical precursors or compounds other than the proteinaceous agent of interest. In a preferred embodiment, an antibody of the invention is isolated. In a specific embodiment, a proteinaceous agent (e.g., a peptide, polypeptide, fusion protein, or antibody) is “purified” by applying the steps described in FIG. 1 to separate the proteinaceous agent from other materials.

As used herein, the term “isolated” or “purified” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment, a nucleic acid molecule encoding an antibody of the invention is isolated.

As used herein, the term “isolated” or “purified” in the context of an organic or inorganic molecule (whether it be a small or large molecule), other than a proteinaceous agent or a nucleic acid, refers to an organic or inorganic molecule substantially free of a different organic or inorganic molecule. Preferably, an organic or inorganic molecule is 60%, 65%, 70%, 75%, 80%, 85%, 90%, 95%, 99%, 99.5%, 99.9%, or 100% free of a second, different organic or inorganic molecule. In a preferred embodiment, an organic and/or inorganic molecule is isolated or purified.

The phrase “low to undetectable levels of aggregation” as used herein refers to samples containing no more than 5%, no more than 4%, no more than 3%, no more than 2%, no more than 1% and most preferably no more than 0.5% aggregation by weight of protein as measured by high performance size exclusion chromatography (HPSEC).

The term “low to undetectable levels of fragmentation” as used herein refers to samples containing equal to or more than 80%, 85%, 90%, 95%, 98% or 99% of the total protein, for example, in a single peak as determined by HPSEC, or in two peaks (heavy- and light-chains) by reduced Capillary Gel Electrophoresis (rCGE), representing the non-degraded antibody or a non-degraded fragment thereof, and containing no other single peaks having more than 5%, more than 4%, more than 3%, more than 2%, more than 1%, or more than 0.5% of the total protein in each. The term “reduced Capillary Gel Electrophoresis” as used herein refers to capillary gel electrophoresis under reducing conditions sufficient to reduce disulfide bonds in an antibody or fragment thereof.

As used herein, the terms “manage”, “managing” and “management” refer to the beneficial effects that a subject derives from a therapy (e.g., a prophylactic or therapeutic agent), which does not result in a cure of the disease. In certain embodiments, a subject is administered one or more therapies (e.g., prophylactic or therapeutic agents) to “manage” a disorder so as to prevent the progression or worsening of the disorder.

The terms “non-responsive” and “refractory” as used herein describe patients treated with a currently available therapy (such as but not limited to, a prophylactic or therapeutic agent) for a disease or disorder (e.g., an inflammatory disorder, an autoimmune disorder, a disorder associated with aberrant expression and/or activity of integrin α_{V}β₃, a disorder associated with abnormal bone metabolism, a disorder associated with aberrant angiogenesis, or cancer) which is not clinically adequate to relieve one or more symptoms associated with the disease or disorder. Typically, such patients suffer from severe, persistently active disease and require additional therapy to ameliorate the symptoms associated with their disease or disorder.

The phrase “pharmaceutically acceptable” as used herein means approved by a regulatory agency of the Federal or a state government, or listed in the U.S. Pharmacopeia, European Pharmacopia or other generally recognized pharmacopeia for use in animals, and more particularly in humans.

The term “polyol” as used herein refers to a sugar that contains many —OH groups compared to a normal saccharide.

The terms “prophylactic agent” and “prophylactic agents” as used refer to any agent(s) which can be used in the prevention of a disease or disorder (e.g., an autoimmune disorder, an inflammatory disorder, a disorder associated with abnormal bone metabolism, a disorder associated with abnormal angiogenesis, or cancer) or one or more symptoms thereof. In certain embodiments, the term “prophylactic agent” refers to an antibody or antibody fragment that immunospecifically binds to integrin α_{V}β₃ (e.g., VITAXIN®). In accordance with these embodiments, the antibody or antibody fragment may be a component of a liquid formulation of the invention. In certain other embodiments, the term “prophylactic agent” does not refer to an antibody or antibody fragment that immunospecifically binds to integrin α_{V}β₃ (e.g., VITAXIN®). Preferably, a prophylactic agent is an agent which is known to be useful to, or has been or is currently being used to the prevent or impede the onset, development, progression, and/or severity of a disease or disorder (e.g., of an autoimmune disorder, an inflammatory disorder, a cancer, a disorder characterized by abnormal bone metabolism, a disorder characterized by aberrant expression and/or activity of integrin α_{V}β₃ or a disorder characterized by abnormal angiogenesis). Prophylactic agents may be characterized as different agents based upon one or more effects the agents have in vitro and/or in vivo. For example, an anti-angiogenic agent may also be characterized as an immunomodulatory agent.

The terms “prevent”, “preventing” and “prevention” as used herein refer to the prevention of the recurrence, onset, or development of a disease or disorder (e.g., an autoimmune disorder, an inflammatory disorder, a disorder characterized by abnormal bone metabolism, a disorder characterized by aberrant expression and/or activity of integrin α_{V}β₃, a disorder characterized by abnormal angiogenesis, or a cancer) or one or more symptoms thereof in a subject resulting from the administration of a therapy (e.g., a prophylactic or therapeutic agent), or combination therapies (e.g., the administration of a combination of prophylactic agents).

The phrase “prophylactically effective amount” as used herein refers to the amount of a therapy (e.g., a prophylactic agent (e.g., an antibody or fragment thereof that immunospecifically binds to integrin α_{V}β₃ or the amount of a liquid formulation of the invention comprising said antibody or antibody fragment)), which is sufficient to result in the prevention of the development, recurrence, onset, or progression of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) or one or more symptoms thereof, or to enhance or improve the prophylactic effect(s) of another therapy (e.g., a prophylactic agent).

The term “saccharide” as used herein refers to a class of molecules that are derivatives of polyhydric alcohols. Saccharides are commonly referred to as carbohydrates and may contain different amounts of sugar (saccharide) units, e.g., monosaccharides, disaccharides and polysaccharides.

The phrase “side effects” as used herein encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky.

Side effects from administration of REMICADE™ include, but are not limited to, risk of serious infection and hypersensitivity reactions. Other side effects range from nonspecific symptoms such as fever or chills, pruritus or urticaria, and cardiopulmonary reactions such as chest pain, hypotension, hypertension or dyspnea, to effects such as myalgia and/or arthralgia, rash, facial, hand or lip edema, dysphagia, sore throat, and headache. Yet other side effects include, but are not limited to, abdominal hernia, splenic infarction, splenomegaly, dizziness, upper motor neuron lesions, lupus erythematosus syndrome, rheumatoid nodules, ceruminosis, abdominal pain, diarrhea, gastric ulcers, intestinal obstruction, intestinal perforation, intestinal stenosis, nausea, pancreatitis, vomiting, back pain, bone fracture, tendon disorder or injury, cardiac failure, myocardial ischema, lymphoma, thrombocytopenia, cellulitis, anxiety, confusion, delirium, depression, somnolence, suicide attempts, anemia, abscess, bacterial infections, and sepsis. Side effects from administration of ENBREL™ include, but are not limited to, risk of serious infection and sepsis, including fatalities. Adverse side effects range from serious infections such as pyelonephritis, bronchitis, septic arthritis, abdominal abscess, cellulitis, osteomyelitis, wound infection, pneumonia, foot abscess, leg ulcer, diarrhea, sinusitis, sepsis, headache, nausea, rhinitis, dizziness, pharyngitis, cough, asthenia, abdominal pain, rash, peripheral edema, respirator disorder, dyspepsia, sinusitis, vomiting, mouth ulcer, alopecia, and pheumonitis to other less frequent adverse effects such as heart failure, myocardial infarction, myocardia ischemia, cerebral ischemia, hypertension, hypotension, cholecystitis, pancreatitis, gastrointestinal hemorrhage, bursitis, depression, dyspnea, deep vein thrombosis, pulmonary embolism, membranous glomerulonephropathy, polymyositis, and thrombophlebitis. The side effects resulting from administration of methotrexate include, but are not limited to, serious toxic reactions, which can be fatal, such as unexpectedly severe bone marrow suppression, gastrointestinal toxicity, hepatotoxicity, fibrosis and cirrhosis after prolonged use, lung diseases, diarrhea and ulcerative stomatitis, malignant lymphomas and occasionally fatal severe skin reactions.

Side effects from chemotherapy include, but are not limited to, gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Other side effects include gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (56th ed., 2002 and 57th ed., 2003).

The term “small molecule” and analogous terms include, but are not limited to, peptides, peptidomimetics, amino acids, amino acid analogs, polynucleotides, polynucleotide analogs, nucleotides, nucleotide analogs, organic or inorganic compounds (i.e., including heterorganic and/or ganometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The terms “stability” and “stable” as used herein in the context of a liquid formulation comprising an antibody or antibody fragment that immunospecifically binds to integrin α_{V}β₃ refer to the resistance of the antibody or antibody fragment in the formulation to degradation or fragmentation under given manufacture, preparation, transportation and storage conditions. The “stable” formulations of the invention retain biological activity under given manufacture, preparation, transportation and storage conditions. The stability of said antibody or antibody fragment can be assessed by degrees of degradation or fragmentation, as measured by HPSEC, compared to a reference formulation. A reference formulation is a reference standard frozen at −70° C. consisting of 10 mg/ml of an antibody or antibody fragment (e.g., VITAXIN®) in histidine-HCl buffer, pH 6.0 that contains 150 mM NaCl, which reference formulation regularly gives a single monomer peak (≧97% area) by HPSEC. Alternatively, a reference formulation is a reference standard frozen at −70° C. consisting of 10 mg/ml of an antibody or antibody fragment (e.g., VITAXIN®) in histidine-HCl buffer at pH 6.0, which reference formulation regularly gives a single monomer peak (≧−97% area) by HPSEC. The overall stability of a formulation comprising an antibody or antibody fragment that immunospecifically binds to integrin α_{V}β₃ can be assessed by various immunological assays including, for example, ELISA and radioimmunoassay using isolated integrin α_{V}β₃ molecules or cells expressing the same.

The terms “subject” and “patient” are used interchangeably herein. As used herein, the terms “subject” and “subjects” refer to an animal, preferably a mammal including a non-primate (e.g., a cow, pig, horse, cat, dog, rat, and mouse) and a primate (e.g., a chimpanzee, a monkey such as a cynomolgous monkey, and a human), and more preferably a human. In one embodiment, the subject is not an immunocompromised or immunosuppressed mammal, preferably a human (e.g., an HIV patient). In another embodiment, the subject is not a mammal, preferably a human, with a lymphocyte count under approximately 500 cells/mm³. In another embodiment, the subject is a mammal, preferably a human, who is or has previously been treated with one or more TNF-α antagonists. In another embodiment, the subject is a mammal, preferably a human, who is or has previously been treated with one or more TNF-α antagonists and methotrexate. In another embodiment, the subject is a mammal, preferably a human, who is not currently being treated with a TNF-α antagonist or methotrexate. In another embodiment, the subject is a mammal, preferably a human, with an inflammatory disorder or an autoimmune disorder that is refractory to treatment with a TNF-α antagonist, a non-steriodal anti-inflammatory agent or methotrexate alone. In yet another embodiment, the subject is a mammal, preferably a human, with cancer that is refractory to treatment with one or more chemotherapeutic agents and/or radiation therapy. In a preferred embodiment, the subject is a human.

The term “substantially free of surfactant” as used herein refers to a formulation of an antibody or antibody fragment that immunospecifically binds to integrin α_{V}β₃, said formulation containing less than 0.0005%, less than 0.0003%, or less than 0.0001% of surfactants.

The term “substantially free of salt” as used herein refers to a formulation of an antibody or antibody fragment that immunospecifically binds to integrin α_{V}β₃, said formulation containing less than 0.0005%, less than 0.0003%, or less than 0.0001% of inorganic salts.

The term “synergistic” as used herein refers to a combination of therapies (e.g., use of prophylactic or therapeutic agents) which is more effective than the additive effects of any two or more single therapy. For example, a synergistic effect of a combination of prophylactic or therapeutic agents permits the use of lower dosages of one or more of the agents and/or less frequent administration of said agents to a subject with a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer). The ability to utilize lower dosages of prophylactic or therapeutic agents and/or to administer said agents less frequently reduces the toxicity associated with the administration of said agents to a subject without reducing the efficacy of said agents in the prevention, management or treatment of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer). In addition, a synergistic effect can result in improved efficacy of therapies in the prevention or treatment of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer). Finally, synergistic effect of a combination of therapies (e.g., prophylactic or therapeutic agents) may avoid or reduce adverse or unwanted side effects associated with the use of any single therapy.

The terms “therapeutic agent” and “therapeutic agents” as used herein refer to any agent(s) which can be used in the treatment, management or amelioration a disease or disorder (e.g., an inflammatory disease, an autoimmune disease, a disorder characterized by abnormal bone metabolism, a disorder characterized by aberrant expression and/or activity of integrin β_{V}β₃, a disorder characterized by abnormal angiogenesis, or cancer) or one or more symptoms thereof. In certain embodiments, the term “therapeutic agent” refers to an antibody or fragment thereof that immunospecifically binds to integrin α_{V}β₃ (e.g., VITAXIN®). In accordance with these embodiments, the antibody or antibody fragment may be a component of a liquid formulation of the invention. In certain other embodiments, the term “therapeutic agent” refers does not refer to an antibody or fragment thereof that immunospecifically binds to integrin α_{V}β₃ (e.g., VITAXIN®). Preferably, a therapeutic agent is an agent which is known to be useful for, or has been or is currently being used for the treatment, management or amelioration of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) or one or more symptoms thereof. Therapeutic agents may be characterized as different agents based upon one or more effects the agents have in vitro and/or in vivo, for example, an inflammatory agent may also be characterized as an immunomodulatory agent.

The term “therapeutically effective amount” as used herein refers to the amount of a therapy (e.g., a therapeutic agent (e.g., an antibody or a fragment thereof, which immunospecifically binds to integrin α_{V}β₃ or a liquid formulation of the invention comprising said antibody or antibody fragment)), which is sufficient to reduce the severity of a disease or disorder, reduce the duration of a disease or disorder, ameliorate one or more symptoms associated with a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer), prevent the advancement of a disease or disorder, cause regression of the disease or disorder, or enhance or improve the therapeutic effect(s) of another therapy (e.g., another therapeutic agent).

As used herein, the terms “therapies” and “therapy” can refer to any protocol(s), method(s) and/or agent(s) that can be used in the prevention, treatment, management or amelioration of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) or one or more symptoms thereof. In certain embodiments, the terms “therapy” and “therapies” refer to chemotherapy, radiation therapy, hormonal therapy, biological therapy, and/or other therapies useful for the treatment of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) known to medical personnel skilled.

The terms “treat”, “treatment” and “treating” as used herein refer to the reduction or amelioration of the progression, severity, and/or duration of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) or the amelioration of one or more symptoms thereof resulting from one or more therapies (including but not limited to, the administration of one or more prophylactic or therapeutic agents, radiation therapy, hormone therapy, surgery, physical therapy, and any other methods or agents that can be used). In certain embodiments, such terms refer to a reduction in the swelling of one or more joints, organs or tissues, or a reduction in the pain associated with an autoimmune or inflammatory disorder resulting from the administration of one or more prophylactic or therapeutic agents to a subject with such a disorder. In other embodiments, such terms refer to a reduction in a human's PASI score. In other embodiments, such terms refer to an improvement in a human's global assessment score. In other embodiments, such terms refer to the inhibition or reduction in the proliferation of cancerous cells, the inhibition or reduction the spread of tumor cells (metastasis), the inhibition or reduction in the onset, development or progression of one or more symptoms associated with cancer, or the reduction in the size of a tumor. In other embodiments, such terms refer to inhibition or reduction in bone loss or bone readsorption. In yet other embodiments, such terms refer to inhibition or reduction in abnormal angiogenesis.

The term “very little to no loss of the biological activities” as used herein refers to antibody activities, including specific binding abilities of antibodies or antibody fragments to integrin α_{V}β₃ as measured by various immunological assays, including, but not limited to ELISAs and radioimmunoassays. In one embodiment, the antibodies or antibody fragments of the formulations of the invention retain approximately 50%, preferably 55%, 60%, 65%, 70%, 75%, 80%, 85%, 90%, 95% or 98% of the ability to immunospecifically bind to integrin α_{V}β₃ (i.e., the vitronectin receptor (VnR)) as compared to a reference antibody or antibody fragment (e.g., VITAXIN®) as measured by an immunological assay known to one of skill in the art or described herein. For example, an ELISA based assay may be used to compare the ability of an antibody or antibody fragment to immunospecifically bind to integrin α_{V}β₃ to a VITAXIN® reference standard. In this assay, referred to as the VnR Binding ELISA, plates are coated with integrin α_{V}β₃ isolated from human placenta and the binding signal of a set concentration of a VITAXIN® reference standard is compared to the binding signal of the same concentration of a test antibody or antibody fragment. A “reference standard” as used herein refers to an antibody or antibody fragment (e.g., VITAXIN®) that is frozen at −70° C. consisting of 10 mg/ml of an antibody or antibody fragment (e.g., VITAXIN®) in histidine-HCl buffer, pH 6.0, and containing 150 mM NaCl, which reference formulation regularly gives a single monomer peak (≧97% area) by HPSEC.

### 4. BRIEF DESCRIPTION OF THE FIGURES

FIG. 1 is a schematic diagram showing the outline for preparing purified antibodies that immunospecifically bind to integrin α_{V}β₃.

### 5. DETAILED DESCRIPTION OF THE INVENTION

The liquid formulations of the present invention provide a ready-to-use preparation of an antibody or antibody fragment that immunospecifically binds to integrin α_{V}β₃ for administering to a subject without having to reconstitute the preparation accurately and aseptically and waiting for a period of time until the solution clarifies before administering the formulation to the subject. It simplifies the procedure of administering the formulation to a subject for a healthcare professional. Furthermore, due to its high stability during the storage, the formulations of the present invention can contain an antibody or antibody fragment that immunospecifically binds to integrin α_{V}β₃ at concentrations in the range of about 15 mg/ml to about 300 mg/ml without causing an adverse effect on the biological activities of the antibody or antibody fragment due to protein aggregation and/or fragmentation during a prolonged storage. Such stability not only ensures the efficacy of the antibodies or antibody fragments but also reduces possible risks of causing adverse effects on a subject. Furthermore, the use of fewer components in the formulation results in fewer risks of introducing contamination. In addition, the manufacturing process of the liquid formulations of the present invention is simplified and more efficient than the manufacturing process for the lyophilized version because all stages of the manufacturing of the liquid formulations are carried out in an aqueous solution, involving no drying process, such as lyophilization and freeze-drying. Accordingly, it is more cost effective as well.

5.1. Antibody Formulations

The liquid formulations of the present invention provide antibody formulations which are substantially free of surfactant, inorganic salts, and/or other excipients and yet exhibit high stability during long periods of storage. In a specific embodiment, such antibody formulations are homogeneous. The formulations of the present invention comprise histidine and an antibody or antibody fragment which immunospecifically binds to integrin α_{V}β₃ at concentrations of about 15 mg/ml to about 300 mg/ml. In one embodiment, the formulations of the invention do not comprise other ingredients except for water or suitable solvents. In a specific embodiment, the antibody or antibody fragment that immunospecifically binds to integrin α_{V}β₃ which is included in the liquid formulations of the invention is VITAXIN® or an antigen-binding fragment thereof. In another embodiment, the antibody or antibody fragment that immunospecifically binds to integrin α_{V}β₃ which is included in the liquid formulations of the invention is not VITAXIN® or an antigen-binding fragment thereof. In a preferred embodiment, the antibody or antibody fragment that immunospecifically binds to integrin α_{V}β₃ which is included in the liquid formulations of the invention is an antibody or antibody fragment comprising one or more VH CDRs and/or one or more VL CDRs listed in Table 1, supra. In another embodiment, the antibody or antibody fragment that immunospecifically binds to integrin α_{V}β₃ which is included in the liquid formulations of the invention is an antibody or antibody fragment conjugated to another moiety, including but not limited to, a heterologous polypeptide, another antibody or an antibody fragment, a marker sequence, a diagnostic agent, a therapeutic agent, a radioactive metal ion, a polymer, albumin, and a solid support. In yet another embodiment, liquid formulations of the invention comprise two or more antibodies or antibody fragments that immunospecifically binds to integrin α_{V}β₃, wherein at least one of the antibodies or antibody fragments is VITAXIN® or an antigen-binding fragment thereof.

The concentration of an antibody or antibody fragment that immunospecifically binds to integrin α_{V}β₃ which is included in the liquid formulations of the invention is at least 15 mg/ml, at least 20 mg/ml, at least 25 mg/ml, at least 30 mg/ml, at least 35 mg/ml, at least 40 mg/ml, at least 45 mg/ml, at least 50 mg/ml, at least 55 mg/ml, at least 60 mg/ml, at least 65 mg/ml, at least 70 mg/ml, at least 75 mg/ml, at least 80 mg/ml, at least 85 mg/ml, at least 90 mg/ml, at least 95 mg/ml, at least 100 mg/ml, at least 105 mg/ml, at least 110 mg/ml, at least 115 mg/ml, at least 120 mg/ml, at least 125 mg/ml, at least 130 mg/ml, at least 135 mg/ml, at least 140 mg/ml, at least 150 mg/ml, at least 175 mg/ml, at least 200 mg/ml, at least 250 mg/ml, at least 275 mg/ml, or at least 300 mg/ml. In a specific embodiment, the concentration of an antibody or antibody fragment that immunospecifically binds to integrin α_{V}β₃ which is included in the liquid formulations of the invention is about 75 mg/ml, about 100 mg/ml, about 125 mg/ml, about 150 mg/ml, about 175 mg/ml, about 200 mg/ml, about 225 mg/ml, about 250 mg/ml, about 275 mg/ml, or about 300 mg/ml. In another embodiment, the concentration of an antibody or antibody fragment that immunospecifically binds to integrin α_{V}β₃ which is included in the liquid formulation of the invention is between 15-500 mg/ml, between 50-300 mg/ml, between 50-250 mg/ml, between 50-200 mg/ml, between 50-175 mg/ml, between 50-150 mg/ml, between 50-125 mg/ml, or between 50-100 mg/ml.

The concentration of histidine which is included in the liquid formulations of the invention ranges from 1 mM to 100 mM, preferably 5 mM to 50 mM, and more preferably 10 mM to about 25 mM. In a specific embodiment, the concentration of histidine which is included in the liquid formulations of the invention is 5 mM, 10 mM, 15 mM, 20 mM, 25 mM, 30 mM, 35 mM, 40 mM, 45 mM, or 50 mM. Histidine can be in the form of L-histidine, D-histidine, or a mixture thereof, but L-histidine is the most preferable. Histidine can be also in the form of hydrates. Histidine may be used in a form of pharmaceutically acceptable salt, such as hydrochloride (e.g., monohydrochloride and dihydrochloride), hydrobromide, sulfate, acetate, etc. The purity of histidine should be at least 98%, preferably at least 99%, and most preferably at least 99.5%. As used herein, the term “purity” in the context of histidine refers to chemical purity of histidine as understood in the art, e.g., as described in The Merck Index, 13^{th} ed., O'Neil et al. ed. (Merck & Co., 2001).

The pH of the formulation should not be equal to the isoelectric point of the particular antibody or antibody fragment to be used in the formulation (e.g., the isoelectric point of VITAXIN® ranges from 8.65 to 8.89) and may range from about 5.0 to about 7.0, preferably about 5.5 to about 6.5, more preferably about 5.8 to about 6.2, and most preferably about 6.0.

In addition to histidine and an antibody or antibody fragment that immunospecifically binds to integrin α_{V}β₃, the formulations of the present invention may further comprise glycine at a concentration of less than 150 mM, less than 100 mM, less than 75 mM, less than 50 mM, less than 25 mM, less than 10 mM, less than 5.0 mM, or less than 2.0 mM. In a specific embodiment, the formulations of the present invention further comprise glycine at a concentration of 1-150 mM, 1-100 mM, 1-75 mM, 1-50 mM, 1-25 mM, 1-10 mM, 1-5.0 mM, or 1-2.0 mM. The amount of glycine in the formulation should not cause a significant buffering effect so that antibody precipitation at its isoelectric point can be avoided. Glycine may be also used in a form of pharmaceutically acceptable salt, such as hydrochloride, hydrobromide, sulfate, acetate, etc. The purity of glycine should be at least 98%, preferably at least 99%, and most preferably 99.5%. As used herein, the term “purity” in the context of glycine refers to chemical purity of glycine as understood in the art, e.g., as described in The Merck Index, 13^{th} ed., O'Neil et al. ed. (Merck & Co., 2001). In a specific embodiment, glycine is not included in the formulations of the present invention.

Optionally, the formulations of the present invention may further comprise other excipients, such as saccharides (e.g., sucrose, mannose, trehalose, etc.) and polyols (e.g., mannitol, sorbitol, etc.). In one embodiment, the other excipient is a saccharide. In a specific embodiment, the saccharide is sucrose, which is at a concentration ranging from between about 1% to about 20%, preferably about 5% to about 15%, and more preferably about 8% to 10% of the formulation. In another embodiment, the saccharide is sucrose, which is at a concentration of 1%, 3%, 5%, 8%, 10%, 15%, or 20% of the formulation. In another embodiment, the other excipient is a polyol. Preferably, however, the liquid formulations of the present invention do not contain mannitol. In a specific embodiment, the polyol is polysorbate (e.g., Tween 20), which is at a concentration ranging from between about 0.001% to about 1%, preferably, about 0.01% to about 0.1% of the formulation. In a specific embodiment, the polyol is polysorbate (e.g., Tween 20), which is at a concentration of 0.001%, 0.005%, 0.01%, 0.02%, 0.05%, 0.08%, 0.1%, 0.5%, or 1% of the formulation.

The liquid formulations of the present invention exhibit stability at the temperature range of 38° C.-42° C. for at least 15 days and, in some embodiments, not more than 25 days, at the temperature range of 20° C.-24° C. for at least 6 months, at the temperature range of 2° C.-8° C. (in particular, at 4° C.) or at least 6 months, at least 1 year, at least 1.5 years, at least 2 years, at least 2.5 years, at least 3 years or at least 4 years, and at the temperature of −20° C. for at least 2 years, at least 3 years, at least 4 years, or at least 5 years, as assessed by high performance size exclusion chromatography (HPSEC). Namely, the liquid formulations of the present invention have low to undetectable levels of aggregation and/or fragmentation, as defined herein, after the storage for the defined periods as set forth above. Preferably, no more than 5%, no more than 4%, no more than 3%, no more than 2%, no more than 1%, and most preferably no more than 0.5% of the antibody or antibody fragment forms an aggregate as measured by HPSEC, after the storage for the defined periods as set forth above. Furthermore, liquid formulations of the present invention exhibit almost no loss in biological activities of the antibody or antibody fragment during the prolonged storage under the condition described above, as assessed by various immunological assays including, for example, enzyme-linked immunosorbent assay (ELISA) and radioimmunoassay to measure the ability of the antibody or antibody fragment to immunospecifically bind to integrin α_{V}β₃. The liquid formulations of the present invention retain after the storage for the above-defined periods more than 80%, more than 85%, more than 90%, more than 95%, more than 98%, more than 99%, or more than 99.5% of the initial biological activities (e.g., the ability to bind to integrin αvβ3) of the formulation prior to the storage. In some embodiments, the liquid formulations of the present invention retain after the storage for the above-defined periods at least 80%, at least 85%, at least 90%, at least 95%, at least 98%, at least 99%, or at least 99.5% of the biological activities (e.g., the ability to bind to integrin αvβ3) compared to a reference antibody representing the antibody prior to the storage.

The liquid formulations of the present invention can be prepared as unit dosage forms. For example, a unit dosage per vial may contain 1 ml, 2 ml, 3 ml, 4 ml, 5 ml, 6 ml, 7 ml, 8 ml, 9 ml, 10 ml, 15 ml, or 20 ml of different concentrations of an antibody or antibody fragment that immunospecifically binds to integrin α_{V}β₃ ranging from about 15 mg/ml to about 300 mg/ml, about 50 mg/ml to about 300 mg/ml, about 75 mg/ml to about 300 mg/ml, about 95% mg/ml to about 300 mg/ml, about 100 mg/ml to about 300 mg/ml, about 150 mg/ml to about 300 mg/ml, about 200 mg/ml to about 300 mg/ml, about 100 mg/ml to about 200 mg/ml, about 100 mg/ml to about 150 mg/ml, or about 100 mg/ml to about 175 mg/ml. If necessary, these preparations can be adjusted to a desired concentration by adding a sterile diluent to each vial.

The invention encompasses stable liquid formulations comprising a single antibody or antibody fragment that immunospecifically binds to integrin α_{V}β₃. The invention also encompasses stable liquid formulations comprising two or more antibodies or antibody fragments that immunospecifically bind to integrin α_{V}β₃. In a specific embodiment, a stable liquid formulation of the invention comprises VITAXIN® or a fragment thereof that immunospecifically binds to integrin α_{V}β₃. In another embodiment, a stable liquid formulation of the invention comprises two or more antibodies or antibody fragments that immunospecifically bind to integrin α_{V}β₃, wherein one of the antibodies or antibody fragments is VITAXIN® or an antigen-binding fragment thereof. In an alternative embodiment, a stable liquid formulation of the invention comprises two or more antibodies or antibody fragments that immunospecifically bind to integrin α_{V}β₃, with the proviso that the antibodies or antibody fragments do not include VITAXIN® or an antigen-binding fragment thereof.

5.1.1. Antibodies Immunospecific for Integrin α_{V}β₃

It should be recognized that antibodies that immunospecifically bind to integrin α_{V}β₃ and function as antagonists are known in the art. Examples of known antibodies that immunospecifically bind to integrin α_{V}β₃ include, but are not limited to, 11D2 (Searle), the murine monoclonal LM609 (Scripps, International Publication Nos. WO 89/05155 and U.S. Pat. No. 5,753,230, which is incorporated herein by reference in its entirety), the humanized monoclonal antibody MEDI-522 (a.k.a. VITAXIN®, MedImmune, Inc., Gaithersburg, Md.; Wu et al., 1998, PNAS USA 95(11):6037-6042; International Publication Nos WO 98/33919 and WO 00/78815, each of which is incorporated herein by reference in its entirety), 17661-37E and 17661-37E 1-5 (USBiological), MON 2032 and 2033 (CalTag), ab7166 (BV3) and ab 7167 (BV4) (Abcam), and WOW-1 (Kiosses et al., Nature Cell Biology, 3:316-320).

Antibodies that immunospecifically bind to integrin αᵥβ₃ include, but are not limited to, monoclonal antibodies, bispecific antibodies, multispecific antibodies, human antibodies, humanized antibodies, chimeric antibodies, camelised antibodies, single-chain Fvs (scFv), single chain antibodies, disulfide-linked Fvs (sdFv), and anti-idiotypic (anti-Id) antibodies (including, e.g., anti-Id antibodies to antibodies of the invention). In particular, antibodies of the present invention include immunoglobulin molecules and immunologically active portions of immunoglobulin molecules, i.e., molecules that contain an antigen binding site that immunospecifically binds to integrin αᵥβ₃. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. In a preferred embodiment, antibodies and antibody fragments that immunospecifically bind to integrin αᵥβ₃ are antagonists of integrin αᵥβ₃. In one embodiment, antibodies and antibody fragments that immunospecifically bind to integrin αᵥβ₃ are not antagonists of integrin αvβ1, αvβ5, αvβ6, or αvβ8. In another preferred embodiment, antibodies and antibody fragments that immunospecifically bind to integrin αᵥβ₃ inhibit or reduce angiogenesis. In one embodiment, antibodies and antibody fragments that immunospecifically bind to integrin αᵥβ₃ inhibit or reduce angiogenesis at least 90%, at least 80%, at least 70%, at least 60%, at least 50%, at least 40%, at least 30%, at least 20%, at least 10%, or at least 5% relative to a negative control using an assay known in the art, e.g., Chick Chorioallantoic Membrane (CAM) angiogenesis assay (see, e.g., Nguyen et al., Microvascular Res. 1994, 47:31-40); and Matrigel Plug Assay (see, e.g., Kragh et al., International J. of Oncology, 22:305-311 (2003)).

The antibodies and antibody fragments that immunospecifically bind to integrin αᵥβ₃ may be from any animal origin including birds and mammals (e.g., human, murine, donkey, sheep, rabbit, goat, guinea pig, camel, horse, or chicken). Preferably, the antibodies that immunospecifically bind to integrin αᵥβ₃ are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies that immunospecifically bind to integrin αvβ₃ may be monospecific, bispecific, trispecific or of greater multispecificity. Multispecific antibodies may be specific for different epitopes of integrin αvβ₃ or may be specific for both an integrin αvβ₃ epitope as well as for a heterologous epitope, such as a heterologous polypeptide or solid support material. See, e.g., International Patent Publication Nos WO 93/17715, WO 92/08802, WO 91/00360, and WO 92/05793; Tutt, et al., J. Immunol. 147:60-69 (1991); U.S. Pat. Nos. 4,474,893, 4,714,681, 4,925,648, 5,573,920, and 5,601,819; and Kostelny et al., J. Immunol. 148:1547-1553 (1992).

The present invention encompasses antibodies and antibody fragments that have a high binding affinity for integrin αvβ₃. In a specific embodiment, an antibody or antibody fragment that immunospecifically binds to integrin αvβ₃ has an association rate constant or kₒₙ rate (antibody (Ab)+antigen (Ag)^{k}^{ₒₙ}→Ab-Ag) of at least 10⁵ M⁻¹ s⁻¹, at least 5×10⁵ M⁻¹ s⁻¹, at least 10⁶ M⁻¹ s⁻¹, at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹. In a preferred embodiment, an antibody or antibody fragment that immunospecifically binds to integrin αᵥβ₃ has a kₒₙ of at least 2×10⁵ M⁻¹ s⁻¹, at least 5×10⁵ M⁻¹ s⁻¹, at least 10⁶ M⁻¹ s⁻¹, at least 5×10⁶ M⁻¹ s⁻¹, at least 10⁷ M⁻¹ s⁻¹, at least 5×10⁷M⁻¹ s⁻¹, or at least 10⁸ M⁻¹ s⁻¹.

In another embodiment, an antibody or antibody fragment that immunospecifically binds to integrin αvβ₃ has a k_{off} rate (antibody (Ab)+antigen (Ag)^{K}^{_{off}}←Ab-Ag) of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹. In a preferred embodiment, an antibody or antibody fragment that immunospecifically binds to integrin αvβ₃ has a kₒₙ of less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹.

In another embodiment, an antibody or antibody fragment that immunospecifically binds to integrin αvβ₃ has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In yet another embodiment, an antibody or antibody fragment that immunospecifically binds to integrin αvβ₃ has a dissociation constant or K_{d} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M.

In a specific embodiment, an antibody or antibody fragment that immunospecifically binds to integrin αᵥβ₃ is LM609 or an antigen-binding fragment thereof (e.g., one or more complementarity determining regions (CDRs) of LM609). LM609 has the amino acid sequence disclosed, e.g., in International Publication No. WO 89/05155 (which is incorporated herein by reference in its entirety), or the amino acid sequence of the monoclonal antibody produced by the cell line deposited with the American Type Culture Collection (ATCC®), 10801 University Boulevard, Manassas, Va. 20110-2209 as Accession Number HB 9537. In an alternative embodiment, an antibody that immunospecifically binds to integrin αvβ₃ is not LM609 or an antigen-binding fragment of LM609.

In a preferred embodiment, an antibody that immunospecifically binds to integrin αvβ₃ is VITAXIN® or an antigen-binding fragment thereof (e.g., one or more CDRs of VITAXIN®). VITAXIN® is disclosed, e.g., in International Publication Nos. WO 98/33919, WO 00/78815, and WO 02/070007, and U.S. application Ser. No. 09/339,222, each of which is incorporated herein by reference in its entirety. In an alternative embodiment, an antibody that immunospecifically binds to integrin αᵥβ₃ is not VITAXIN® or an antigen-binding fragment of VITAXIN®.

The present invention also encompasses antibodies and antibody fragments that immunospecifically bind integrin αvβ₃, said antibodies and antibody fragments comprising a variable heavy (“VH”) domain having an amino acid sequence of the VH domain for LM609 or VITAXIN®. The present invention also provides antibodies and antibody fragments that immunospecifically bind to integrin αᵥβ₃, said antibodies and antibody fragments comprising a VH CDR having an amino acid sequence of any one of the VH CDRs listed in Table 1.

In one embodiment, antibodies or antibody fragments that immunospecifically bind to integrin αᵥβ₃ comprise a VH CDR1 having the amino acid sequence of SEQ ID NO:1. In another embodiment, antibodies and antibody fragments that immunospecifically bind to integrin αᵥβ₃ comprise a VH CDR2 having the amino acid sequence of SEQ ID NO:2. In another embodiment, antibodies and antibody fragments that immunospecifically bind to integrin αᵥβ₃ comprise a VH CDR3 having the amino acid sequence of SEQ ID NO:3. In another embodiment, antibodies or antibody fragments that immunospecifically bind to integrin αᵥβ₃ comprise a VH CDR1 having the amino acid sequence of SEQ ID NO:1 and a VH CDR2 having the amino acid sequence of SEQ ID NO:2. In another embodiment, antibodies and antibody fragments that immunospecifically bind to integrin αᵥβ₃ comprise a VH CDR1 having the amino acid sequence of SEQ ID NO:1 and a VH CDR3 having the amino acid sequence of SEQ ID NO:3. In another embodiment, antibodies and antibody fragments that immunospecifically bind to integrin αᵥβ₃ comprise a VH CDR2 having the amino acid sequence of SEQ ID NO:2 and a VH CDR3 having the amino acid sequence of SEQ ID NO:3. In a preferred embodiment, antibodies that immunospecifically bind to integrin αᵥβ₃ comprise a VH CDR1 having the amino acid sequence of SEQ ID NO:1, a VH CDR2 having the amino acid sequence of SEQ ID NO:2, and a VH CDR3 having the amino acid sequence of SEQ ID NO:3.

The present invention also encompasses antibodies and antibody fragments that immunospecifically bind to integrin αᵥβ₃, said antibodies and antibody fragments comprising a variable light (“VL”) domain having an amino acid sequence of the VL domain for LM609 or VITAXIN®. The present invention also provides antibodies and antibody fragments that immunospecifically bind to integrin αᵥβ₃ said antibodies and antibody fragments comprising a VL CDR having an amino acid sequence of any one of the VL CDRs listed in Table 1.

In one embodiment, antibodies or antibody fragments that immunospecifically bind to integrin αᵥβ₃ comprise a VL CDR1 having the amino acid sequence of SEQ ID NO:4. In another embodiment, antibodies or antibody fragments that immunospecifically bind to integrin αᵥβ₃ comprise a VL CDR2 having the amino acid sequence of SEQ ID NO:5. In another embodiment, antibodies or antibody fragments that immunospecifically bind to integrin αᵥβ₃ comprise a VL CDR3 having the amino acid sequence of SEQ ID NO:6. In another embodiment, antibodies or antibody fragments that immunospecifically bind to integrin αᵥβ₃ comprise a VL CDR1 having the amino acid sequence of SEQ ID NO:4 and a VL CDR2 having the amino acid sequence of SEQ ID NO:5. In another embodiment, antibodies or antibody fragments that immunospecifically bind to integrin αᵥβ₃ comprise a VL CDR1 having the amino acid sequence of SEQ ID NO:4 and a VL CDR3 having the amino acid sequence of SEQ ID NO:6. In another embodiment, antibodies or antibody fragments that immunospecifically bind to integrin αᵥβ₃ comprise a VL CDR2 having the amino acid sequence of SEQ ID NO:5 and a VL CDR3 having the amino acid sequence of SEQ ID NO:6. In a preferred embodiment, antibodies or antibody fragments that immunospecifically bind to integrin αᵥβ₃ comprise a VL CDR1 having the amino acid sequence of SEQ ID NO:4, a VL CDR2 having the amino acid sequence of SEQ ID NO:5, and a VL CDR3 having the amino acid sequence of SEQ ID NO:6.

The present invention also encompasses antibodies and antibody fragments that immunospecifically bind to integrin αᵥβ₃, said antibodies comprising a VH domain disclosed herein combined with a VL domain disclosed herein, or other VL domain. The present invention further provides antibodies and antibody fragments that immunospecifically bind to integrin αᵥβ₃, said antibodies and antibody fragments comprising a VL domain disclosed herein combined with a VH domain disclosed herein, or other VH domain.

The present invention also encompasses antibodies and antibody fragments that immunospecifically bind to integrin αᵥβ₃, said antibodies and antibody fragments comprising one or more VH CDRs and one or more VL CDRs listed in Table 1. In particular, the invention provides for an antibody that immunospecifically binds to integrin αᵥβ₃, said antibody or antibody fragment comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 1 supra.

In one embodiment, an antibody or antibody fragment that immunospecifically binds to integrin αᵥβ₃ comprises a VH CDR1 having the amino acid sequence of SEQ ID NO:1 and a VL CDR1 having the amino acid sequence of SEQ ID NO:4. In another embodiment, an antibody or antibody fragment that immunospecifically binds to integrin αᵥβ₃ comprises a VH CDR1 having the amino acid sequence of SEQ ID NO:1 and a VL CDR2 having the amino acid sequence of SEQ ID NO:5. In another embodiment, an antibody or antibody fragment that immunospecifically binds to integrin αᵥβ₃ comprises a VH CDR1 having the amino acid sequence of SEQ ID NO:1 and a VL CDR3 having the amino acid sequence of SEQ ID NO:6.

In another embodiment, an antibody or antibody fragment that immunospecifically binds to integrin αᵥβ₃ comprises a VH CDR2 having the amino acid sequence of SEQ ID NO:2 and a VL CDR1 having the amino acid sequence of SEQ ID NO:4. In another embodiment, an antibody or antibody fragment that immunospecifically binds to integrin αᵥβ₃ comprises a VH CDR2 having the amino acid sequence of SEQ ID NO:2 and a VL CDR2 having the amino acid sequence of SEQ ID NO:5. In another embodiment, an antibody or antibody fragment that immunospecifically binds to integrin αᵥβ₃ comprises a VH CDR2 having the amino acid sequence of SEQ ID NO:2 and a VL CDR3 having the amino acid sequence of SEQ ID NO:6.

In another embodiment, an antibody or antibody fragment that immunospecifically binds to integrin αᵥβ₃ comprises a VH CDR3 having the amino acid sequence of SEQ ID NO:3 and a VL CDR1 having the amino acid sequence of SEQ ID NO:4. In another embodiment, an antibody or antibody fragment that immunospecifically binds to integrin αᵥβ₃ comprises a VH CDR3 having the amino acid sequence of SEQ ID NO:3 and a VL CDR2 having the amino acid sequence of SEQ ID NO:5. In a preferred embodiment, an antibody or antibody fragment that immunospecifically binds to integrin αᵥβ₃ comprises a VH CDR3 having the amino acid sequence of SEQ ID NO:3 and a VL CDR3 having the amino acid sequence of SEQ ID NO:6.

The present invention also encompasses a nucleic acid molecule, generally isolated, encoding an antibody or antibody fragment that immunospecifically binds to integrin αᵥβ₃. In a specific embodiment, an isolated nucleic acid molecule encodes an antibody that immunospecifically binds to integrin αᵥβ₃, said antibody or antibody fragment having the amino acid sequence of LM609 or VITAXIN®. In one embodiment, an isolated nucleic acid molecule encodes an antibody or antibody fragment that immunospecifically binds to integrin αᵥβ₃, said antibody or antibody fragment comprising a VH domain having the amino acid sequence of the VH domain of LM609 or VITAXIN®. In another embodiment, an isolated nucleic acid molecule encodes an antibody or antibody fragment that immunospecifically binds to integrin αᵥβ₃ said antibody or antibody fragment comprising a VH domain having the amino acid sequence of the VH domain of the monoclonal antibody produced by the cell line deposited with the ATCC® as Accession Number HB 9537. In another embodiment, an isolated nucleic acid molecule encodes an antibody or antibody fragment that immunospecifically binds to integrin αᵥβ₃ said antibody or antibody fragment comprising a VL domain having the amino acid sequence of the VL domain of LM609 or VITAXIN®. In another embodiment, an isolated nucleic acid molecule encodes an antibody or antibody fragment that immunospecifically binds to integrin αᵥβ₃, said antibody or antibody fragment comprising a VL domain having the amino acid sequence of the VL domain of the monoclonal antibody produced by the cell line deposited with the ATCC® as Accession Number HB 9537.

The invention encompasses an isolated nucleic acid molecule(s) encoding an antibody or antibody fragment that immunospecifically binds to integrin αᵥβ₃ said antibody or antibody fragments comprising a VH CDR having the amino acid sequence of any of the VH CDRs listed in Table 1, supra. In particular, the invention encompasses an isolated nucleic acid molecule(s) encoding an antibody or antibody fragment that immunospecifically binds to integrin αᵥβ₃, said antibody or antibody fragment comprising one, two, three, four, five or more VH CDRs having the amino acid sequence of any of the VH CDRs listed in Table 1, supra. In one embodiment, an isolated nucleic acid molecule encodes an antibody or antibody fragment that immunospecifically binds to integrin αᵥβ₃ said antibody or antibody fragments comprising a VH CDR1 having the amino acid sequence of the VH CDR1 listed in Table 1, supra. In another embodiment, an isolated nucleic acid molecule encodes an antibody or antibody fragment that immunospecifically binds to integrin αᵥβ₃, said antibody or antibody fragments comprising a VH CDR2 having the amino acid sequence of the VH CDR2 listed in Table 1. In yet another embodiment, an isolated nucleic acid molecule encodes an antibody or antibody fragment that immunospecifically binds to integrin αᵥβ₃, said antibody or antibody fragment comprising a VH CDR3 having the amino acid sequence of the VH CDR3 listed in Table 1.

The present invention encompasses an isolated nucleic acid molecule(s) encoding an antibody or antibody fragment that immunospecifically binds to integrin αᵥβ₃, said antibody or antibody fragments comprising a VL CDR having an amino acid sequence of any of the VL CDRs listed in Table 1, supra. In particular, the invention provides an isolated nucleic acid molecule encoding an antibody or antibody fragment that immunospecifically binds to integrin αᵥβ₃ said antibody or antibody fragment comprising one, two, three or more VL CDRs having the amino acid sequence of any of the VL CDRs listed in Table 1, supra. In one embodiment, an isolated nucleic acid molecule encodes an antibody or antibody fragment that immunospecifically binds to integrin αᵥβ₃ said antibody or antibody fragment comprising a VL CDR1 having the amino acid sequence of the VL CDR1 listed in Table 1. In another embodiment, an isolated nucleic acid molecule encodes an antibody or antibody fragment that immunospecifically bind to integrin αᵥβ₃, said antibody or antibody fragment comprising a VL CDR2 having the amino acid sequence of the VL CDR2 listed in Table 1. In yet another embodiment, an isolated nucleic acid molecule encodes an antibody or antibody fragment that immunospecifically binds to integrin αᵥβ₃ said antibody comprising a VL CDR3 having the amino acid sequence of the VL CDR3 listed in Table 1.

In another embodiment, an isolated nucleic acid molecule encodes an antibody or antibody fragment that immunospecifically binds to integrin αᵥβ₃, said antibody or antibody fragment comprising a VH domain having the amino acid sequence of the VH domain of LM609 or VITAXIN® and a VL domain having the amino acid sequence of the VL domain of LM609 or VITAXIN®. In another embodiment, an isolated nucleic acid molecule encodes an antibody or antibody fragment that immunospecifically binds to integrin αᵥβ₃, said antibody or antibody fragment comprising a VH CDR1, a VL CDR1, a VH CDR2, a VL CDR2, a VH CDR3, a VL CDR3, or any combination thereof having an amino acid sequence listed in Table 1.

The present invention also encompasses antibodies and antibody fragments that immunospecifically bind to integrin αᵥβ₃, said antibodies and antibody fragments comprising derivatives of the VH domains, VH CDRs, VL domains, or VL CDRs described herein that immunospecifically bind to integrin αᵥβ₃. Standard techniques known to those of skill in the art can be used to introduce mutations (e.g., additions, deletions, and/or substitutions) in the nucleotide sequence encoding an antibody of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues (i.e., amino acid residues which are not critical for the antibody to immunospecifically bind to integrin αᵥβ₃). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody or antibody fragment can be expressed and the activity of the antibody or antibody fragments can be determined.

The present invention encompasses antibodies and antibody fragments that immunospecifically bind to integrin αᵥβ₃, said antibodies and antibody fragments comprising the amino acid sequence of LM609 or VITAXIN® with one or more amino acid residue substitutions in the variable light (VL) domain and/or variable heavy (VH) domain. The present invention also encompasses antibodies and antibody fragments that immunospecifically bind to integrin αᵥβ₃ said antibodies and antibody fragments comprising the amino acid sequence of LM609 or VITAXIN® with one or more amino acid residue substitutions in one or more VL CDRs and/or one or more VH CDRs. The antibody or antibody fragment engineered to contain substitutions in the VH domain, VH CDRs, VL domain and/or VL CDRs of LM609 or VITAXIN® can be tested in vitro and/or in vivo, for example, for its ability to bind to integrin αᵥβ₃ (by, e.g., immunoassays including, but not limited to ELISAs and BIAcore), or for its ability to prevent, treat, manage or ameliorate an autoimmune, an inflammatory disorder, a disorder associated with abnormal bone metabolism, a disorder associated with aberrant angiogenesis, a disorder associated with aberrant expression and/or activity of integrin αᵥβ₃ or cancer, or a symptom thereof.

In a specific embodiment, an antibody or antibody fragment that immunospecifically binds to integrin αᵥβ₃ is encoded by a nucleotide sequence that hybridizes to the nucleotide sequence encoding the monoclonal antibody produced by the cell line deposited with the ATCC® as Accession Number 1B 9537 under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In a specific embodiment, an antibody or antibody fragment that immunospecifically binds to integrin αᵥβ₃ is encoded by a nucleotide sequence that hybridizes to the nucleotide sequence encoding the LM609 or VITAXIN® under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

In a specific embodiment, an antibody or antibody fragment that immunospecifically binds to integrin αᵥβ₃ comprises an amino acid sequence of a VH domain and/or an amino acid sequence a VL domain encoded by a nucleotide sequence that hybridizes to the nucleotide sequence encoding the VH and/or VL domains of LM609 or VITAXIN® under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In another embodiment, an antibody or antibody fragment that immunospecifically binds to integrin αᵥβ₃ comprises an amino acid sequence of a VH CDR or an amino acid sequence of a VL CDR encoded by a nucleotide sequence that hybridizes to the nucleotide sequence encoding any one or more of the VH CDRs or VL CDRs listed in Table 1 under stringent conditions e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody or antibody fragment that immunospecifically binds to integrin αᵥβ₃ comprises an amino acid sequence of a VH CDR or an amino acid sequence of a VL CDR encoded by a nucleotide sequence that hybridizes to the nucleotide sequence encoding any one of VH CDRs or VL CDRs of the monoclonal antibody produced by the cell line deposited with the ATCC® as Accession Number HB 9537 under stringent conditions e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody or antibody fragment that immunospecifically binds to integrin αᵥβ₃ comprises an amino acid sequence of a VH CDR and an amino acid sequence of a VL CDR encoded by nucleotide sequences that hybridizes to the nucleotide sequences encoding any one of the VH CDRs and VL CDRs listed in Table 1 under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody or antibody fragment that immunospecifically binds to integrin αᵥβ₃ comprises an amino acid sequence of a VH CDR and an amino acid sequence of a VL CDR encoded by nucleotide sequences that hybridizes to the nucleotide sequences encoding the monoclonal antibody produced by the cell line deposited with the ATCC® as Accession Number HB 9537 under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In a specific embodiment, an antibody or antibody fragment that immunospecifically binds to integrin αᵥβ₃ comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of the monoclonal antibody produced by the cell line deposited with the ATCC® as Accession Number HB 9537. In another embodiment, an antibody that immunospecifically binds to integrin αᵥβ₃ comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of VITAXIN®. The determination of percent identity of two amino acid sequences can be determined by any method known to one skilled in the art, including BLAST protein searches.

In another embodiment, an antibody or antibody fragment that immunospecifically binds to integrin αᵥβ₃ comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of VITAXIN®. In another embodiment, an antibody or antibody fragment that immunospecifically binds to integrin αᵥβ₃ comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of the monoclonal antibody produced by the cell line deposited with the ATCC® as Accession Number HB 9537.

In another embodiment, an antibody or antibody fragment that immunospecifically binds to integrin αᵥβ₃ comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VH CDRs listed in Table 1. In another embodiment, an antibody or antibody fragment that immunospecifically binds to integrin αᵥβ₃ comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of one of the VH CDRs of the monoclonal antibody produced by the cell line deposited with the ATCC® as Accession Number HB 9537.

In another embodiment, an antibody or antibody fragment that immunospecifically binds to integrin αᵥβ₃ comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of VITAXIN®. In another embodiment, an antibody or antibody fragment that immunospecifically binds to integrin αᵥβ₃ comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of the monoclonal antibody produced by the cell line deposited with the ATCC® as Accession Number HB 9537.

In another embodiment, an antibody or antibody fragment that immunospecifically binds to integrin αᵥβ₃ comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 1. In another embodiment, an antibody or antibody fragment that immunospecifically binds to integrin αᵥβ₃ comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs of the monoclonal antibody produced by the cell line deposited with the ATCC® as Accession Number HB 9537.

The present invention encompasses antibodies and antibody fragments that compete with an antibody described herein for binding to integrin αᵥβ₃. In a specific embodiment, the present invention encompasses antibodies and antibody fragments that compete with LM609 or an antigen-binding fragment thereof for binding to integrin αᵥβ₃. In a preferred embodiment, the present invention encompasses antibodies and antibody fragments that compete with VITAXIN® or an antigen-binding fragment thereof for binding to integrin αᵥβ₃.

The present invention also encompasses proteins, polypeptides, or peptides comprising (or alternatively consisting of) a VH domain that competes with the VH domain of LM609 or VITAXIN™, or a protein, polypeptide, or peptide comprising (or alternatively consisting of) a VH domain of LM609 or VITAXIN™ for binding to integrin αᵥβ₃. The present invention also encompasses proteins, polypeptides, or peptides comprising (or alternatively consisting of) a VL domain that competes with a VL domain of LM609 or VITAXIN™, or a protein, polypeptide, or peptide comprising a VL domain of LM609 or VITAXIN™ for binding to integrin αᵥβ₃.

The present invention also encompasses proteins, polypeptides, or peptides comprising (or alternatively consisting of) at least one VH CDR that competes with a VH CDR listed in Table 1 or a protein, polypeptide, or peptide comprising a VH CDR listed in Table 1 for binding to integrin αᵥβ₃, or a VH CDR of the monoclonal antibody produced by the cell line deposited with the ATCC™ as Accession Number HB 9537 for binding to integrin αᵥβ₃. The present invention also encompasses proteins, polypeptides, or peptides comprising (or alternatively consisting of) at least one VL CDR that competes with a VL CDR listed in Table 1 or a protein, polypeptide or peptide comprising a VL CDR listed in Table 1 for binding to integrin αᵥβ₃, or a VL CDR of the monoclonal antibody produced by the cell line deposited with the ATCC™ as Accession Number HB 9537 for binding to integrin αᵥβ₃.

Antibodies or antibody fragments that immunospecifically bind to integrin αᵥβ₃ include derivatives that are modified, i.e, by the covalent attachment of any type of molecule to the antibody or antibody fragment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also encompasses antibodies that immunospecifically bind to integrin αvβ₃, said antibodies and antibody fragments comprising a framework region known to those of skill in the art. In a non-limiting example, framework regions are generated or derived from a human germline immunoglobulin sequence using a method known to those of skill in the art, e.g., using polymerase chain reaction (PCR). Human germline immunoglobulin sequences can be found, e.g., at the NCBI website (see also, Kawasaki et al., 2001, Eur. J. Immunol. 31:1017-1028; Schable and Zachau, 1993, Biol. Chem. Hoppe Seyler 374:1001-1022; Matsuda et al., 1998, J. Exp. Med., 188:1973-1975). Preferably, the fragment region of an antibody of the invention is human. In a specific embodiment, an antibody that immunospecifically binds to integrin αvβ₃ comprises the framework region of VITAXIN®.

The present invention also encompasses antibodies and antibody fragments which immunospecifically bind to integrin αvβ₃, said antibodies comprising the amino acid sequence of VITAXIN® with one or more mutations (e.g., one or more amino acid substitutions) in the framework regions. In certain embodiments, antibodies and antibody fragments which immunospecifically bind to integrin αvβ₃ comprise the amino acid sequence of VITAXIN® with one or more amino acid residue substitutions in the framework regions of the VH and/or VL domains.

The present invention also encompasses antibodies and antibody fragments which immunospecifically bind to integrin αvβ₃, said antibodies and antibody fragments comprising the amino acid sequence of VITAXIN® with one or more mutations (e.g., one or more amino acid residue substitutions) in the variable and framework regions.

The present invention also encompasses antibodies and antibody fragments which immunospecifically bind to integrin αvβ₃, said antibodies and antibody fragments comprising constant regions known to those of skill in the art. See e.g., Wu & An, 2003, Methods Mol. Biol., 207, 213-233; Wu, 2003, Methods Mol. Biol., 207, 197-212; International Publication No. WO 86/05807; International Publication No. WO 89/01036; and U.S. Pat. No. 5,122,464. Preferably, the constant regions of an antibody of the invention are human.

The present invention also encompasses fusion proteins comprising an antibody or antibody fragment that immunospecifically binds to integrin αvβ₃ and a heterologous polypeptide. Preferably, the heterologous polypeptide that the antibody or antibody fragment is fused to is useful for targeting the antibody or antibody fragment to platelets, monocytes, macrophages, endothelial cells, and/or B cells.

5.1.1.1. Methods of Identifying Antibodies Immunospecific for Integrin αᵥβ₃

The invention provides methods for identifying antibodies and antibody fragments that are immunospecific for integrin α_{V}β₃, particularly for antibodies and antibody fragments that specifically bind to the same epitope as VITAXIN® and/or LM609. Mutation of residues 171, 173 and/or 174 of the human β₃ chain have been found to disrupt binding of VITAXIN® and/or LM609 antibodies to the integrin α_{V}β₃ heterodimer. Although VITAXIN® and LM609 do not bind to mouse integrin α_{V}β₃, it has been found that VITAXIN® and LM609 do bind to a modified mouse integrin α_{V}β₃ in which the region of the mouse β chain that corresponds to amino acids 164-202 of the human β chain are replaced with amino acids 164-202 of the human β chain. In certain embodiments, amino acid substitutions are made in the subunits of integrin α_{V}β₃ for example to change the ligand specificity of the integrin α_{V}β₃ and/or disrupt the heterodimerization of the subunit chains. Preferably the integrin α_{V}β₃ is human. In specific embodiments, such amino acid substitutions disrupt the specific interaction of certain antagonists of integrin α_{V}β₃ with a particular integrin α_{V}β₃ epitope. In a preferred embodiment, the amino acid substitutions are made within regions of an integrin subunit that confers ligand binding specificity, preferably ligand binding specificity of LM609 and/or VITAXIN®, particularly residues 164-202 of human β₃. Alternatively, mouse β chain residues corresponding to residues 164-202 of the human β₃ chain are replaced with the residues 164-202 of the human β₃ chain. Such mouse-human chimeras can be used to screen for antagonists that bind to the region 164-202 of human β₃ but not to mouse integrin α_{V}β₃.

In preferred embodiments, the amino acid substitutions are made in the β₃ subunit. In certain embodiments, human β₃ residues are substituted with rat residues as described in Table 2. In one embodiment, the substitution of human residue Glu to rat residue Gln at position 171 (“Mutation A”) disrupts integrin α_{V}β₃ binding to LM609. This same change disrupts binding to VITAXIN®. In another embodiment, the substitution of human residue Leu and Glu to rat residues Ile, and Lys at positions 173 and 174, respectively (“Mutation B”) both disrupt binding to VITAXIN® and increase binding to an anti rat β₃ antibody. In yet another embodiment, the substitution of human residues Asp and Thr to rat residues Thr and Ser at positions 179 and 182 respectively (“Mutation C”) confer binding specificity to an anti-rat β₃ antibody. Mutations A and C combined (three substituted residues) confer binding specificity for the mouse-anti-rat β₃ antibody and disrupts binding to VITAXIN®. In a specific preferred embodiment, amino acids 171, 173 and 174 can be substituted to disrupt binding to VITAXIN®. In an alternate preferred embodiment, amino acids 171, 173, 174, 179 and 182 can be substituted to disrupt binding of integrin α_{V}β₃ to LM609 and humanized anti-integrin α_{V}β₃ antibodies such as VITAXIN®. Such substitutions preferred examples but not limiting. Such substituted subunits are merely exemplary and not limiting. Any integrin α_{V}β₃ regions identified to be responsible for antibody binding can be altered with substituted, deleted or inserted residues to characterize binding specificity of various antibodies and to screen for antibodies with the same a similar binding specificity.

Amino acid substituted subunits of integrin α_{V}β₃ can be used for screening antibodies with specific affinity for particular epitopes by identifying monoclonal antibodies that bind to wild type integrin α_{V}β₃ but not the altered form, or that bind mouse α_{V}β₃ integrins with a region substituted with the corresponding region from the human α_{V}β₃ but do not bind to wild type mouse integrin α_{V}β₃. In addition, the invention provides methods for identifying monoclonal antibodies that bind to the heterodimerized α_{V}β₃ but not the α_{V} or the β₃ chains when not included in a heterodimer. Such screening can be accomplished by any routine method for assaying antibody specificity known in the art, for example, using cell lines that do not express wild type integrin α_{V}β₃ to recombinantly express the mutant α_{V}β₃ or individual α_{V} or β₃ chains. The antibodies identified from such screening methods can be useful for the prevention, management and treatment of integrin α_{V}β₃-mediated diseases and disorders, including but not limited to inflammatory diseases, autoimmune diseases, bone metabolism related disorder, angiogenic related disorders, disorders related to aberrant expression and/or activity of α_{V}β₃, and cancer. It is also contemplated that such antibodies can be used in the methods and compositions contemplated by the present invention. Preferably, these antibodies are not LM609, VITAXIN® or an antibody or antibody binding fragment thereof having the CDRs (or one, two, three, four or five of the CDRs or CDR3 of the heavy chain) of LM609 or VITAXIN® with no more than one, no more than two, no more than five, no more than eight, or no more than ten amino acid substitutions, deletions or insertions.

TABLE 2

| Human Beta3 | Mutation A | Mutation B | | Mutation C | |
|---|---|---|---|---|---|
| mutants | (Glu-Gln) | (Leu-Ile), (Glu-Lys) | | (Asp-Thr), (Thr-Ser) | |
| A1(A, C) | E171Q | | | D179T | T182S |
| A6 | E171Q | | | | |
| B1 | | L173 I | E174K | | |
| C14 | | | | D179T | T182S |
| C16 | | | | D179T | T182S |
| ABC17 | E171Q | L173 I | E174K | D179T | T182S |

5.1.1.2. Antibodies Having Increased Half-Lives that Immunospecifically Bind to Integrin αᵥβ₃

The present invention provides for antibodies and antibody fragments that immunospecifically bind to integrin αᵥβ₃ which have a extended half-life in vivo. In particular, the present invention provides antibodies and antibody fragments that immunospecifically bind to integrin αᵥβ₃ which have a half-life in an animal, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies and single chain antibodies) or antibody fragments (e.g., Fab fragments) in vivo, for example, inert polymer molecules such as high molecular weight polyethyleneglycol (PEG) can be attached to the antibodies or antibody fragments with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies or antibody fragments can be tested for binding activity as well as for in vivo efficacy using methods known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication No. WO 98/23289; International Publication No. WO 97/34631; and U.S. Pat. No. 6,277,375, each of which is incorporated herein by reference in its entirety.

Further, antibodies or antibody fragments can be conjugated to albumin in order to make the antibody or antibody fragment more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

5.1.1.3. Antibody Conjugates

The present invention encompasses the use of antibodies or antibody fragments conjugated to one or more moieties, including but not limited to, peptides, polypeptides, proteins, fusion proteins, nucleic acid molecules, small molecules, mimetic agents, synthetic drugs, inorganic molecules, and organic molecules.

The present invention encompasses the use of antibodies or antibody fragments recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies or antibody fragments may be used to target heterologous polypeptides to particular cell types, either in vitro or in vivo, by fusing or conjugating the antibodies or antibody fragments to antibodies specific for particular cell surface receptors. Antibodies or antibody fragments fused or conjugated to heterologous polypeptides may also be used in in vitro immunoassays and purification methods using methods known in the art. See e.g., International Publication No. WO 93/21232; European Patent No. EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated herein by reference in their entireties.

The present invention further includes compositions comprising heterologous proteins, peptides or polypeptides fused or conjugated to antibody fragments. For example, the heterologous polypeptides may be fused or conjugated to an antigen-binding fragment of an antibody (e.g., a Fab fragment, Fd fragment, Fv fragment, F(ab)₂ fragment, a VH domain, a VL domain, a VH CDR, a VL CDR, or fragment thereof). Methods for fusing or conjugating polypeptides to antibody fragments are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references incorporated by reference in their entireties).

Additional fusion proteins, e.g., of VITAXIN® or other anti-integrin α_{V}β₃ antibodies or antibody fragments, may be generated through the techniques of gene-shuffling, motif-shuffling, exon-shuffling, and/or codon-shuffling (collectively referred to as “DNA shuffling”). DNA shuffling may be employed to alter the activities of antibodies of the invention (e.g., antibodies or antibody fragments with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33 Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or antibody fragments, or the encoded antibodies or antibody fragments, may be altered by being subjected to random mutagenesis by error-prone PCR, random nucleotide insertion or other methods prior to recombination. One or more portions of a polynucleotide encoding an antibody or antibody fragment, which portions immunospecifically bind to integrin α_{V}β₃ may be recombined with one or more components, motifs, sections, parts, domains, fragments, etc. of one or more heterologous molecules.

Moreover, the antibodies or antibody fragments can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies or antibody fragments of the present invention (including variants thereof) conjugated to a diagnostic or detectable agent. Such antibodies can be useful for monitoring or prognosing the onset, the development, progression and/or severity of an inflammatory disease, an autoimmune disease, a disorder associated with aberrant expression and/or activity of integrin αvβ3, a disorder associated with abnormal bone metabolism, a disorder associated with aberrant angiogenesis, or cancer, as part of a clinical testing procedure, such as determining the efficacy of a particular therapy. Such diagnosis and detection can be accomplished by coupling the antibody or antibody fragment to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I), carbon (¹⁴C), sulfur (³⁵S), tritium (³H), indium (¹¹⁵In, ¹¹³In, ¹¹²In, ¹¹¹In), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, non-radioactive paramagnetic metal ions, and molecules that are radiolabelled or conjugated to specific radioisotopes.

The present invention further encompasses uses of antibodies or antibody fragment conjugated to a therapeutic moiety. An antibody or antibody fragment may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic moieties include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), Auristatin molecules (e.g., auristatin PHE, bryostatin 1, solastatin 10, see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference), anti-mitotic agents (e.g., vincristine and vinblastine), hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)), and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459), farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305), topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate); HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

Further, an antibody may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), an anti-angiogenic agent, e.g., angiostatin, endostatin or a component of the coagulation pathway (e.g., tissue factor); or, a biological response modifier such as, for example, a lymphokine (e.g., interferon gamma (“IFN-γ”), interleukin-1 (“IL-1”), interleukin-2 (“IL-2”), interleukin-5 (“IL-5”), interleukin-6 (“IL-6”), interleukin-7 (“IL-7”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid. fibrinopeptides A and B from the α and β chains of fibrinogen, fibrin monomer).

Moreover, an antibody or antibody fragment can be conjugated to therapeutic moieties such as a radioactive metal ion, such as alpha-emitters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies or antibody fragment are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody or antibody fragment can be conjugated to a second antibody to form an antibody heteroconjugate as described by Segal in U.S. Pat. No. 4,676,980, which is incorporated herein by reference in its entirety.

Antibodies and antibody fragments may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The therapeutic moiety or drug conjugated to an antibody or antibody fragment that immunospecifically binds to integrin α_{V}β₃ should be chosen to achieve the desired prophylactic or therapeutic effect(s) for a particular disorder in a subject. A clinician or other medical personnel should consider the following when deciding on which therapeutic moiety or drug to conjugate to an antibody or antibody fragment that immunospecifically binds to integrin α_{V}β₃: the nature of the disease, the severity of the disease, and the condition of the subject.

5.2. Method of Preparing the Antibody Formulations

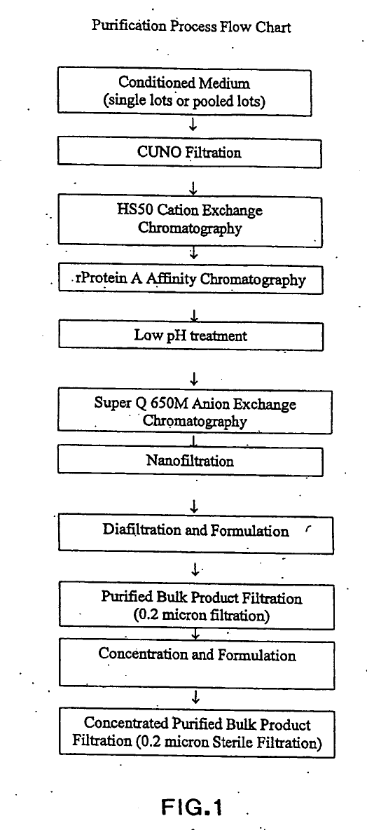

The present invention provides methods for preparing liquid formulations of antibodies or derivatives, analogues, or fragments thereof that immunospecifically bind to integrin α_{V}β₃ FIG. 1 is a schematic diagram showing the outline for preparing purified anti-integrin α_{V}β₃ antibodies. The methods for preparing liquid formulations of the present invention comprise: purifying the antibody or antibody fragment from conditioned medium (either single lots or pooled lots of medium) and concentrating a fraction of the purified antibody or antibody fragment to a final concentration of from about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 150 mg/ml, about 175 mg/ml, about 200 mg/ml, about 250 mg/ml, or about 300 mg/ml. Conditioned medium containing the antibody or antibody fragment that immunospecifically binds to integrin α_{V}β₃ is subjected to CUNO filtration and the filtered antibody is subjected to HS50 cation exchange chromatography. The fraction from the HS50 cation exchange chromatography is then subjected to rProtein A affinity chromatography followed by low pH treatment. Following low pH treatment, the antibody or antibody fragment fraction is subject to super Q 650 anion exchange chromatography and then nanofiltration. The fraction of the antibody or antibody fragment obtained after nanofiltration is then subjected to diafiltration to concentrate the antibody or antibody fragment fraction into the formulation buffer using the same membrane.

The formulation buffer of the present invention comprises histidine at a concentration ranging from about 1 mM to about 100 mM, about 5 mM to about 50 mM, about 10 mM to about 30 mM, or about 10 mM to about 25 mM. In a specific embodiment, the formulation buffer of the present invention comprises histidine at a concentration of about 10 mM, about 12 mM, about 15 mM, about 20 mM or about 25 mM. The formulations may further comprise glycine at a concentration of less than 150 mM, less than 100 mM, less than 75 mM, less than 50 mM, less than 10 mM, less than 3.0 mM, or less than 2.0 mM. The amount of glycine in the formulation should not cause a significant buffering in order to avoid antibody precipitation at its isoelectric point. The pH of the formulation may range from about 5.0 to about 7.0, preferably about 5.5 to about 6.5, more preferably about 5.8 to about 6.2, and most preferably about 6.0. To obtain an appropriate pH for a particular antibody, it is preferable that histidine (and glycine, if added) is first dissolved in water to obtain a buffer solution with higher pH than the desired pH and then the pH is brought down to the desired level by adding HCl. This way, the formation of inorganic salts (e.g., formation of NaCl when, for example, histidine hydrochloride is used as histidine and pH is raised to a desired level by adding NaOH) can be avoided.

The liquid formulations of the present invention can be prepared as unit dosage forms by preparing a vial containing an aliquot of the liquid formulation for a one-time use. For example, a unit dosage per vial may contain 1 ml, 2 ml, 3 ml, 4 ml, 5 ml, 6 ml, 7 ml, 8 ml, 9 ml, 10 ml, 15 ml, or 20 ml of different concentrations of an antibody or a fragment thereof that immunospecifically binds to integrin α_{V}β₃ ranging from about 15 mg/ml to about 300 mg/ml. If necessary, these preparations can be adjusted to a desired concentration by adding a sterile diluent to each vial.

The liquid formulations of the present invention may be sterilized by various sterilization methods, including sterile filtration, radiation, etc. In a most preferred embodiment, the difiltrated antibody formulation is filter-sterilized with a presterilized 0.2 micron filter. Sterilized liquid formulations of the present invention may be administered to a subject to prevent, treat or ameliorate a disease or disorder (e.g., an inflammatory disorder, an autoimmune disorder, a disorder associated with abnormal bone metabolism, a disorder associated with aberrant expression and/or activity of integrin αvβ₃, a disorder associated with aberrant angiogenesis or cancer) or one or more symptoms thereof.

Although the invention is directed to liquid non-lyophilized formulations, it should be noted for the purpose of equivalents that the formulations of the invention may be lyophilized if desired. Thus, the invention encompasses lyophilized forms of the formulations of the invention although such lyophilized formulations are not preferred.

5.3. Methods of Preparing Antibodies

The antibodies and antibody fragments that immunospecifically bind to an antigen can be produced by any method known in the art for the synthesis of antibodies, in particular, by chemical synthesis or preferably, by recombinant expression techniques.

Polyclonal antibodies immunospecific for an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981), and Harlow et al., Using Antibodies: A laboratory Manual, Cold Spring Harbor Laboratory Press (1999) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a RIMMS (repetitive immunization multiple sites) technique can be used to immunize an animal (Kilpatrack et al., 1997, Hybridoma 16:381-9, incorporated herein by reference in its entirety). The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/O1 134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, 5,969,108, 6,33,187, 5,824,520, and 5,702,892; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Completely human antibodies and humanized antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immuoglobulin. A humanized antibody comprises substantially all of at least one, and typically two, variable domains (Fab, Fab′, F(ab′)₂, Fabc, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and most preferably greater than 95%. Humanized antibody can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119-25 (2002), Caldas et al., Protein Eng. 13(5):353-60 (2000), Morea et al., Methods 20(3):267-79 (2000), Baca et al., J. Biol. Chem. 272(16):10678-84 (1997), Roguska et al., Protein Eng. 9(10):895-904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Single domain antibodies, for example, antibodies lacking the light chains, can be produced by methods well-known in the art. See Riechmann et al., 1999, J. Immuno. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety.

Further, the antibodies that immunospecifically bind to an antigen (e.g., integrin αvβ3) can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

5.3.1. Polynucleotide Sequences Encoding an Antibody

The invention provides polynucleotides comprising a nucleotide sequence encoding an antibody or antibody fragment that immunospecifically binds to an antigen. The invention also encompasses polynucleotides that hybridize under high stringency, intermediate or lower stringency hybridization conditions, e.g., as defined supra, to polynucleotides that encode an antibody of the invention.

The polynucleotides may be obtained, and the nucleotide sequence of the polynucleotides determined, by any method known in the art. The nucleotide sequence of antibodies or antibody fragments immunospecific for a desired antigen can be obtained, e.g., from the literature or a database such as GenBank. Since the amino acid sequences of VITAXIN® is known, nucleotide sequences encoding this antibody or a fragment thereof (e.g., a CDR) can be determined using methods well known in the art, i.e., nucleotide codons known to encode particular amino acids are assembled in such a way to generate a nucleic acid that encodes the antibody. Such a polynucleotide encoding the antibody or antibody fragment may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or antibody fragment may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody or antibody fragment is determined, the nucleotide sequence of the antibody or antibody fragment may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to a particular antigen. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

5.3.2. Recombinant Expression of an Antibody

Recombinant expression of an antibody of the invention, derivative, analog or fragment thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well-known in the art. See, e.g., U.S. Pat. No. 6,331,415, which is incorporated herein by reference in its entirety. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication No. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies which immunospecifically bind to integrin α_{V}β₃ is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoters, enhancers, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk−, hgprt− or aprt− cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

5.4. Methods of Monitoring the Stability and Aggregation of Antibody Formulations

There are various methods available for assessing the stability of protein formulations, including antibody formulations, based on the physical and chemical structures of the proteins as well as on their biological activities. For example, to study denaturation of proteins, methods such as charge-transfer absorption, thermal analysis, fluorescence spectroscopy, circular dichroism, NMR, and HPSEC, are available. See, for example, Wang et al., 1988, J. of Parenteral Science & Technology 42(Suppl):S4-S26.

The rCGE and HPSEC are the most common and simplest methods to assess the formation of protein aggregates, protein degradation, and protein fragmentation. Accordingly, the stability of the liquid formulations of the present invention may be assessed by these methods.

For example, the stability of the liquid formulations of the present invention may be evaluated by HPSEC or rCGE, wherein the percent area of the peaks represents the non-degraded antibody or non-degraded antibody fragments. In particular, approximately 250 μg of the antibody or antibody fragment that immunospecifically binds to integrin α_{V}β₃ (approximately 25 μl of a liquid formulation comprising 10 mg/ml said antibody or antibody fragment) is injected onto a TosoH Biosep TSK G3000SW_{XL} column (7.8 mm×30 cm) fitted with a TSK SW ×1 guard column (6.0 mm CX 4.0 cm). The antibody or antibody fragment is eluted isocratically with 0.1 M disodium phosphate containing 0.1 M sodium sulfate and 0.05% sodium azide, at a flow rate of 0.8 to 1.0 ml/min. Eluted protein is detected using UV absorbance at 280 nm. VITAXIN® reference standard is run in the assay as a control, and the results are reported as the area percent of the product monomer peak compared to all other peaks excluding the included volume peak observed at approximately 12 to 14 minutes. Peaks eluting earlier than the monomer peak are recorded as percent aggregate.

The liquid formulations of the present invention exhibit low to undetectable levels of aggregation as measured by HPSEC or rCGE, that is, no more than 5%, no more than 4%, no more than 3%, no more than 2%, no more than 1%, and most preferably no more than 0.5% aggregate by weight protein, and low to undetectable levels of fragmentation, that is, 80% or higher, 85% or higher, 90% or higher, 95% or higher, 98% or higher, or 99% or higher, or 99.5% or higher of the total peak area in the peak(s) representing intact antibodies or fragments thereof. In the case of SDS-PAGE, the density or the radioactivity of each band stained or labeled with radioisotope can be measured and the % density or % radioactivity of the band representing non-degraded antibodies or fragments thereof can be obtained.

The stability of the liquid formulations of the present invention can be also assessed by any assays which measures the biological activity of the antibody or fragments thereof in the formulation. The biological activities of antibodies or antibody fragments include, but are not limited to, antigen-binding activity, complement-activation activity, Fc-receptor binding activity, and so forth. Antigen-binding activity of the antibodies or antibody fragments can be measured by any method known to those skilled in the art, including but not limited to ELISA, radioimmunoassay, Western blot, and the like. Complement-activation activity can be measured by a C3a/C4a assay in the system where the antibody which immunospecifically binds to integrin α_{V}β₃ is reacted in the presence of the complement components with the cells expressing the integrin α_{V}β₃. Also see Harlow et al., Antibodies: A Laboratory Manual, (Cold Spring Harbor Laboratory Press, 2nd ed. 1988) (incorporated by reference herein in its entirety). An ELISA based assay, e.g., may be used to compare the ability of an antibody or antibody fragment thereof to immunospecifically bind to integrin α_{V}β₃ to a VITAXIN® reference standard. In this assay, referred to as the VnR Binding ELISA, plates are coated with integrin α_{V}β₃ isolated from human placenta and the binding signal of a set concentration of a VITAXIN® reference standard is compared to the binding signal of the same concentration of a test antibody or antibody fragment.

The purity of the liquid antibody formulations of the invention may be measured by any method well-known to one of skill in the art such as, e.g., HPSEC. The sterility of the liquid antibody formulations may be assessed as follows: sterile soybean-casein digest medium and fluid thioglycollate medium are inoculated with a test liquid antibody formulation by filtering the liquid antibody formulation through a sterile filter having a nominal porosity of 0.45 μm. When using the Sterisure™ or Steritest™ method, each filter device is aseptically filled with approximately 100 ml of sterile soybean-casein digest medium or fluid thioglycollate medium. When using the conventional method, the challenged filter is aseptically transferred to 100 ml of sterile soybean-casein digest medium or fluid thioglycollate medium. The media are incubated at appropriate temperatures and observed three times over a 14 day period for evidence of bacterial or fungal growth.

5.5. Prophylactic and Therapeutic Utility of the Antibody Formulations

The present invention is also directed to antibody-based therapies which involve administering to a subject, preferably a human, the liquid antibody formulations of the present invention for preventing; treating, managing or ameliorating an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer or a symptom thereof. The liquid formulations of the invention comprise an antibody or a fragment thereof at concentrations of from about 15 mg/ml to about 300 mg/ml in a solution containing histidine, which antibody or antibody fragment thereof immunospecifically binds to integrin α_{V}β₃. The liquid formulations of the invention may comprise a single antibody or fragment thereof that immunospecifically binds to integrin α_{V}β₃ (e.g., VITAXIN®). The liquid formulations of the invention may also comprise two or more antibodies or antibody fragments that immunospecifically bind to integrin α_{V}β₃. In a specific embodiment, one of the antibodies or antibody fragments included in such liquid formulations is VITAXIN® or a fragment thereof. In an alternative embodiment, one of the antibodies or antibody fragments included in such liquid formulations is not VITAXIN® or a fragment thereof. In yet another embodiment, the liquid formulations of the invention comprise an antibody or antibody fragment that immunospecifically binds to integrin α_{V}β₃, and the antibody or antibody fragment is also conjugated to another moiety, including but not limited to, a heterologous protein, peptide or polypeptide, another antibody or an antibody fragment, a marker sequence, a diagnostic agent, a therapeutic agent, a radioactive metal ion, and a solid support.

The liquid formulations of the present invention may be used locally or systemically in the body as a therapeutic. Particularly, the liquid formulations of the invention may be used in the prevention, treatment, management and amelioration of diseases or disorders associated with aberrant expression and/or aberrant activity of integrin α_{V}β₃. The formulations of the invention can be used to regulate the activity of cells expressing integrin α_{V}β₃. In a specific embodiment, the formulations of the invention are used to regulate various activities of a body, including but not limited to, angiogenesis, bone metabolism and immune functions. The formulations of the present invention may also be advantageously utilized in combination with one or more other therapies (e.g., one or more other prophylactic or therapeutic agents), preferably therapies useful in the treatment, prevention, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis, cancer or one or more symptoms thereof. When one or more other therapies (e.g, prophylactic or therapeutic agents) are used, they can be administered separately, in any appropriate form and by any suitable route.

A liquid formulation of the invention may be administered to a mammal, preferably a human, concurrently with one or more other therapies (e.g., one or more other prophylactic or therapeutic agents), preferably therapies useful for the prevention, treatment, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis, cancer or one or more symptoms thereof. The term “concurrently” is not limited to the administration of prophylactic or therapeutic agents/therapies at exactly the same time, but rather it is meant that a liquid formulation of the invention and the other agent/therapy are administered to a mammal in a sequence and within a time interval such that the antibody or antibody fragment that immunospecifically binds to integrin α_{V}β₃ contained in the liquid formulation can act together with the other agent/therapy to provide an increased benefit than if they were administered otherwise. For example, a liquid formulation of the invention and one or more other prophylactic or therapeutic agents useful for prevention, treatment, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer may be administered at the same time or sequentially in any order at different points in time; however, if not administered at the same time, they should be administered sufficiently close in time so as to provide the desired therapeutic or prophylactic effect.

In various embodiments, a liquid formulation of the invention and one or more other therapies (e.g., one or more other prophylactic or therapeutic agents), preferably therapies useful for prevention, treatment, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, a liquid formulation of the invention and one or more other therapies (e.g., one or more other prophylactic or therapeutic agents), preferably therapies useful for prevention, treatment, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer are administered within the same patient visit. In other embodiments, a liquid formulation of the invention and one or more other therapies (e.g., one or more other prophylactic or therapeutic agents), preferably therapies useful for prevention, treatment, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer are administered at about 2 to 4 days apart, at about 4 to 6 days apart, at about 1 week part, at about 1 to 2 weeks apart, or more than 2 weeks apart. In preferred embodiments, a liquid formulation of the invention and one or more other therapies (e.g., prophylactic or therapeutic agents), preferably therapies useful for prevention, treatment, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer are administered in a time frame where both agents are still active. One skilled in the art would be able to determine such a time frame by determining the half-life of the administered agents.

In certain embodiments, a liquid formulation of the invention and one or more other therapies (e.g., one or more other prophylactic or therapeutic agents), preferably therapies useful for prevention, treatment, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer are cyclically administered to a subject. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improves the efficacy of the treatment.

In certain embodiments, a liquid formulation of the invention and one or more other therapies (e.g., one or more other prophylactic or therapeutic agents), preferably therapies useful for prevention, treatment, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer are administered in a cycle of less than about 3 weeks, about once every two weeks, about once every 10 days or about once every week. One cycle can comprise the administration of a therapeutic or prophylactic agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, liquid formulation of the invention and one or more other therapies (e.g., prophylactic or therapeutic agents), preferably therapies useful for prevention, treatment, management or amelioration of an inflammatory disorder, an autoimmune disorder, a disorder associated with aberrant expression and/or activity of integrin αᵥβ₃, a disorder associated with abnormal bone metabolism, a disorder associated with aberrant angiogenesis or cancer are administered in metronomic dosing regimens, either by continuous infusion or frequent administration without extended rest periods. Such metronomic administration can involve dosing at constant intervals without rest periods. Typically the prophylactic or therapeutic agents, in particular cytotoxic agents, are used at lower doses. Such dosing regimens encompass the chronic daily administration of relatively low doses for extended periods of time. In preferred embodiments, the use of lower doses can minimize toxic side effects and eliminate rest periods. In certain embodiments, the prophylactic and therapeutic agents are delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months.

In one embodiment, a liquid formulation of the invention is administered in a dosing regimen that maintains the plasma concentration of the antibody or antibody fragment immunospecific for α_{V}β₃ at a desirable level (e.g., about 0.1 to about 100 μg/ml), which continuously blocks the integrin α_{V}β₃ activity. In a specific embodiment, the plasma concentration of the antibody or antibody fragment is maintained at 0.2 μg/ml, 0.5 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, μg/ml, 20 μg/ml, 25 μg/ml, 30 μg/ml, 35 μg/ml, 40 μg/ml, 45 μg/ml or 50 μg/ml. The plasma concentration that is desirable in a subject will vary depending on several factors, including but not limited to, the nature of the disease or disorder, the severity of the disease or disorder and the condition of the subject. Such dosing regimens are especially beneficial in prevention, treatment, management and amelioration of a chronic disease or disorder.

In one embodiment, a liquid formulation of the invention α_{V}β₃ is administered to a subject with a disease or disorder that associated with abnormal bone metabolism using a dosing regimen that maintains the plasma concentration of the an antibody or antibody fragment that immunospecifically binds to integrin α_{V}β₃ at a level that blocks at least 40%, preferably at least 50%, at least 55%, at least 60%, at least 65%, at least 70%, at least 75%, at least 80%, at least 85%, at least 90% or at least 95% of bone resorption. In a specific embodiment, the plasma concentration of the an antibody or antibody fragment that immunospecifically binds to integrin αvβ3 is maintained at about 0.1 μg/ml to about 100 μg/ml in a subject with a disease or disorder that associated with abnormal bone metabolism.

In some embodiments, a liquid formulation of the invention is administered intermittently to a subject, wherein the liquid formulation comprises an antibody or an antibody fragment conjugated to a moiety (e.g., a therapeutic agent or a toxin).

When used in combination with other therapies (e.g., prophylactic and/or therapeutic agents) useful for prevention, treatment, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer, the liquid formulations of the invention and the other therapy can act additively or, more preferably, synergistically. The invention contemplates administration of a liquid formulation of the invention in combination with other therapies (e.g., prophylactic or therapeutic agents) preferably therapies useful for prevention, treatment, management or amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis or cancer by the same or different routes of administration, e.g., oral and parenteral. In certain embodiments, when a liquid formulation of the invention is administered concurrently with one or more therapies (e.g., prophylactic or therapeutic agents) that potentially produce adverse side effects (including, but not limited to, toxicity), the therapies (e.g., prophylactic or therapeutic agents) can advantageously be administered at a dose that falls below the threshold that the adverse side effect is elicited.

5.5.1. Treatment for Disorders Associated with Aberrant Angiogenesis

The liquid formulations of the invention may be administered to a subject in need thereof to prevent, treat, manage, or ameliorate a disease or disorder associated with aberrant angiogenesis or one or more symptoms thereof. The liquid formulations of the invention may also be administered in combination with one or more therapies (e.g., prophylactic or therapeutic agents) to a subject in need thereof to prevent, treat, manage or ameliorate a disease or disorder associated with aberrant angiogenesis or one or more symptoms thereof. Non-limiting examples of such therapies include, but not limited to, anti-inflammatory agents (e.g., non-steroid anti-inflammatory drugs and steroid drugs), bisphosphonates, HG-CoA reductase inhibitors, immunomodulatory agents, and anti-angiogenic agents. In a specific embodiment, a subject administered a liquid formulation of the invention alone or in combination with another therapy (e.g., a prophylactic or therapeutic agent) is refractory to conventional therapies for a particular disorder associated with aberrant angiogenesis.

In a specific embodiment, the invention provides methods of preventing, managing, treating or ameliorating a disorder associated with aberrant angiogenesis or one or more symptoms thereof, said method comprising administering to a subject in need thereof a dose of a prophylactically or therapeutically effective amount of a liquid formulation of the invention. In another embodiment, the invention provides methods of preventing, managing, treating or ameliorating a disorder associated with aberrant angiogenesis or one or more symptoms thereof, said method comprising administering to a subject in need thereof a dose of a prophylactically or therapeutically effective amount of a liquid formulation of the invention and a dose prophylactically or therapeutically effective amount of one or more therapies (e.g., prophylactic or therapeutic agents) other than antibodies or antibody fragments that immunospecifically bind to integrin αᵥβ₃.

The liquid formulations of the invention may be used as first, second, third or fourth line of treatment for a disorder associated with aberrant angiogenesis. The invention provides methods for managing, treating or ameliorating a disorder associated with aberrant angiogenesis or one or more symptoms thereof in a subject refractory to conventional therapies for such a disease, said methods comprising administering to said subject a dose of a prophylactically or therapeutically effective amount of a liquid formulation of the invention. The invention also provides methods for managing, treating or ameliorating a disorder associated with aberrant angiogenesis or one or more symptoms thereof in a subject refractory to existing single agent therapies for such a disease, said methods comprising administering to said subject a dose of a prophylactically or therapeutically effective amount of a liquid formulation of the invention and a dose prophylactically or therapeutically effective amount of one or more therapies (e.g., prophylactic or therapeutic agents) other than antibodies or fragments thereof that immunospecifically bind to integrin αᵥβ₃. The invention also provides alternative methods for the management or treatment of a disorder associated with aberrant angiogenesis where conventional therapies have proven or may prove too toxic, i.e., results in unacceptable or unbearable side effects, for the subject being treated. Further, the invention provides methods for preventing the recurrence of a disorder associated with aberrant angiogenesis in patients that have been treated and have no disease activity by administering a liquid formulation. In a specific embodiment, a subject administered a liquid formulation of the invention alone or in combination with another therapy (e.g., a prophylactic or therapeutic agent) is refractory to conventional therapies for a particular disease or disorder associated with aberrant angiogenesis.

Diseases or disorders that are associated with aberrant angiogenesis include, but not limited to, neoplastic diseases (non-limiting examples are metastases of tumors and leukemia); diseases of ocular neovascularization (non-limiting examples are age-related macular degeneration, diabetic retinopathy, and retinopathy of prematurity, vascular restenosis); skin diseases (non-limiting examples are infantile hemangiomas, verruca vulgaris, psoriasis, basal cell and squamous cell carcinomas, cutaneous melanoma, Kaposi's sarcoma, neurofibromatosis, recessive dystrophic epidermolysis bullosa); arthritis (non-limiting examples are rheumatoid arthritis, ankylosing spondylitis, systemic lupus, psoriatic arthropathy, Reiter's syndrome, and Sjogren's syndrome); gynecologic diseases (non-limiting examples are endometriosis, preeclampsia during pregnancy, carcinoma of the ovary, endometrium and cervix); and cardiovascular diseases (non-limiting examples are formation of atherosclerotic plaques, atherosclerosis and coronary artery disease).

5.5.2. Treatment for Disorders Associated with Aberrant Bone Metabolism

The liquid formulations of the invention may be used to regulate bone metabolism or used to prevent, treat, manage, or ameliorate a disease or disorder associated with aberrant bone metabolism or one or more symptoms thereof. The liquid formulations of the invention may also be administered in combination with one or more therapies (e.g., prophylactic or therapeutic agents) to a subject in need thereof to prevent, manage, treat or ameliorate a disease or disorder associated with aberrant bone metabolism or one or more symptoms. Non-limiting examples of such therapies include, but are not limited to, those listed in section 5.5.2.1 infra. In a specific embodiment, the invention provides methods of preventing, managing, treating or ameliorating a disorder associated with abnormal bone metabolism or one or more symptoms thereof, said method comprising administering to a subject in need thereof a dose of a prophylactically or therapeutically effective amount of a liquid formulation of the invention. In another embodiment, the invention provides methods of preventing, managing, treating or ameliorating a disorder associated with abnormal bone metabolism or one or more symptoms thereof, said method comprising administering to a subject in need thereof a dose of a prophylactically or therapeutically effective amount of a liquid formulation of the invention and a dose prophylactically or therapeutically effective amount of one or more therapies (e.g., prophylactic or therapeutic agents) other than antibodies or antibody fragments that immunospecifically bind to integrin αᵥβ₃.

The liquid formulations of the invention may be used as first, second, third or fourth line of treatment for a disorder associated with abnormal bone metabolism. The invention provides methods for managing, treating or ameliorating a disorder associated with abnormal bone resorption or one or more symptoms thereof in a subject refractory to conventional therapies for such a disease, said methods comprising administering to said subject a dose of a prophylactically or therapeutically effective amount of a liquid formulation of the invention. The invention also provides methods for managing, treating or ameliorating a disorder associated with abnormal bone metabolism or one or more symptoms thereof in a subject refractory to existing single agent therapies for such a disease, said methods comprising administering to said subject a dose of a prophylactically or therapeutically effective amount of a liquid formulation of the invention and a dose prophylactically or therapeutically effective amount of one or more therapies (e.g., prophylactic or therapeutic agents) other than antibodies or fragments thereof that immunospecifically bind to integrin αᵥβ₃. The invention also provides alternative methods for the management or treatment of a disorder associated with abnormal bone metabolism where conventional therapies have proven or may prove too toxic, i.e., results in unacceptable or unbearable side effects, for the subject being treated. Further, the invention provides methods for preventing the recurrence of a disorder associated with abnormal bone metabolism in patients that have been treated and have no disease activity by administering a liquid formulation. In a specific embodiment, a subject administered a liquid formulation of the invention alone or in combination with another therapy (e.g., a prophylactic or therapeutic agent) is refractory to conventional therapies for a particular disease or disorder associated with aberrant bone metabolism. As used herein, the term “aberrant bone metabolism” is used interchangeably with “abnormal bone metabolism”, which refers to bone metabolism that deviates from its normal process, such as but not limited to, resorption of bone tissues and abnormal growth of the osteocytes.

Diseases or disorders that are associated with aberrant bone metabolism include, but not limited to, rickets and osteomalacia; hypercalcemia which can be caused by, but not limited to, primary hyperparathyroidism (e.g., solitary adenomas, multiple endocrine neoplasia), lithium therapy, familial hypocalciuric hypercalcemia, solid tumor with metastases (e.g., breast cancer), solid tumor with humoral mediation of hypercalcemia (e.g., lung or kidney cancer), hematologic malignancies (e.g., multiple myeloma, lymphoma, leukemia), vitamin D intoxication, sarcoidosis and other granulomatous diseases, idopathic hypercalcemia of infancy, hyperthyroidism, vitamin A intoxication, aluminum intoxication, milk-alkali syndrome, and renal failure; hypocalcemia which can be caused by, but not limited to, hereditary hypoparathyroidism, acquired hypoparathyroidism, chronic renal failure, vitamin D deficiency, tumor lysis, rhabdomyolysis, and osteitis fibrosa after parathyroidectomy; osteoporosis; diseases associated with an increased risk of generalized osteoporosis in adults including but not limited to, Turner syndrome, Klinefelter syndrome, anorexia nervosa, hypothalamic amenorrhea, hyperprolactinemia, primary or secondary hypogonadal state, Cushing's syndrome, hyperparathyroidism, thyrotoxicosis, insulin-dependent diabetes mellitus, acromegaly, adrenal insufficiency, malnutrition, parenteral nutrition, malabsorption syndromes, gastrectomy, severe liver disease, pernicious anemia, rheumatoid arthritis, ankylosing spondylitis, chronic otitis media (cholesteatoma-induced bone resorption), hypertrophic pulmonary osteoarthropathy (HPOA), Gorham-Stout disease, multiple myloma, lymphoma and leukemia, malignancy-associated parathyroid hormone related production, mastocytosis, hemophilia, thalassemia, osteogenesis imperfecta, Marfan syndrome, hemochromatosis, hypophosphatasia, glycogen storage diseases, homocystinuria, Ehlers-Danlos syndrome, porphyria, Menkes' syndrome, Epidermolysis bullosa, Chronic obstructive pulmonary disease, scoliosis, multiple sclerosis, sacoidosis and amyloidosis; drug related osteoporosis, which can be caused by, but not limited to, glucocorticoids, cyclosporine, cytotoxic drugs, anticonvulsants, excessive thyroxine, aluminum, gonadotropin-releasing hormone agonists, heparin and lithium; Paget's disease of bone; osteopetrosis (Albers-Schönberg bone disease); pyknodysostosis; osteomyelosclerosis; hereditary hyperphosphatasia; progressive diaphyseal dysplasia (Camurati-Engelmann disease); melorheostosis; osteopoikilosis; hyperostosis frontalis interna; fibrous dysplasia (Mccune-Albright syndrome); spndyloepiphyseal dysplasia; achondroplasia; enchondromatosis; and ostoechondromatosis.

The liquid formulation of the invention may also be administered to a subject in need thereof to prevent, treat, manage or ameliorate a periodontal disease or one or more symptoms thereof. Periodontal diseases include, but not limited to, gingivitis and periodontitis. The liquid formulations of the invention may also be administered in combination with one or more therapies (e.g., prophylactic or therapeutic agents) to a subject in need thereof to prevent, manage, treat or ameliorate a periodontal disease or one or more symptoms thereof. Non-limiting examples of such therapies include, but are not limited to, Mistoral II, folic acid, green tea extract, aloe vera gel, bee proplis, vitamin K1, vitamin E, chamomile, Coenzyme Q10, improvement of oral hygiene, antibiotics (e.g., PERIOSTAT™ (doxycycline hyclate)), CO₂ laser treatment, scaling, root planing, and surgery. In a specific embodiment, the invention provides methods of preventing, managing, treating or ameliorating a periodontal disease or one or more symptoms thereof, said method comprising administering to a subject in need thereof a dose of a prophylactically or therapeutically effective amount of a liquid formulation of the invention. In another embodiment, the invention provides methods of preventing, managing, treating or ameliorating a periodontal disease or one or more symptoms thereof, said method comprising administering to a subject in need thereof a dose of a prophylactically or therapeutically effective amount of a liquid formulation of the invention and a dose prophylactically or therapeutically effective amount of one or more therapies (e.g., prophylactic or therapeutic agents) other than antibodies or antibody fragments that immunospecifically bind to integrin αᵥβ₃.

The liquid formulations of the invention may be used as first, second, third or fourth line of treatment for a periodontal disease. The invention provides methods for managing, treating or ameliorating a periodontal disease or one or more symptoms thereof in a subject refractory to conventional therapies for such a disease, said methods comprising administering to said subject a dose of a prophylactically or therapeutically effective amount of a liquid formulation of the invention. The invention also provides methods for managing, treating or ameliorating a periodontal disease or one or more symptoms thereof in a subject refractory to existing single agent therapies for such a disease, said methods comprising administering to said subject a dose of a prophylactically or therapeutically effective amount of a liquid formulation of the invention and a dose prophylactically or therapeutically effective amount of one or more therapies (e.g., prophylactic or therapeutic agents) other than antibodies or antibody fragments that immunospecifically bind to integrin αᵥβ₃. The invention also provides alternative methods for the management or treatment of a periodontal disease where conventional therapies have proven or may prove too toxic, i.e., results in unacceptable or unbearable side effects, for the subject being treated. Further, the invention provides methods for preventing the recurrence of a periodontal disease in patients that have been treated and have no disease activity by administering a liquid formulation.

The liquid formulation of the invention may also be administered to a subject in need thereof to prevent, treat, manage or ameliorate aseptic loosening of a joint replacement (e.g., hip or knee replacement) or one or more symptoms thereof. The liquid formulations of the invention may also be administered in combination with one or more therapies (e.g., prophylactic or therapeutic agents) to a subject in need thereof to prevent, manage, treat or ameliorate aseptic loosening of a joint replacement or one or more symptoms thereof. Non-limiting examples of such therapies include, but are not limited to, anti-inflammatory agents, bisphosphonates, vitamin D compounds, anti-clotting agents, surgery and physical therapy. In a specific embodiment, the invention provides methods of preventing, managing, treating or ameliorating a aseptic loosening of a joint replacement or one or more symptoms thereof, said method comprising administering to a subject in need thereof a dose of a prophylactically or therapeutically effective amount of a liquid formulation of the invention. In another embodiment, the invention provides methods of preventing, managing, treating or ameliorating aseptic loosening of a joint replacement or one or more symptoms thereof, said method comprising administering to a subject in need thereof a dose of a prophylactically or therapeutically effective amount of a liquid formulation of the invention and a dose prophylactically or therapeutically effective amount of one or more therapies (e.g., prophylactic or therapeutic agents) other than antibodies or antibody fragments that immunospecifically bind to integrin αᵥβ₃.

The liquid formulations of the invention may be used as first, second, third or fourth line of treatment for aseptic loosening of a joint replacement. The invention provides methods for managing, treating or ameliorating aseptic loosening of a joint replacement or one or more symptoms thereof in a subject refractory to conventional therapies for such a disease, said methods comprising administering to said subject a dose of a prophylactically or therapeutically effective amount of a liquid formulation of the invention. The invention also provides methods for managing, treating or ameliorating aseptic loosening of a joint replacement or one or more symptoms thereof in a subject refractory to existing single agent therapies for such a disease, said methods comprising administering to said subject a dose of a prophylactically or therapeutically effective amount of a liquid formulation of the invention and a dose prophylactically or therapeutically effective amount of one or more therapies (e.g., prophylactic or therapeutic agents other than antibodies or antibody fragments that immunospecifically bind to integrin αᵥβ₃. The invention also provides alternative methods for the management or treatment of aseptic loosening of a joint replacement where conventional therapies have proven or may prove too toxic, i.e., results in unacceptable or unbearable side effects, for the subject being treated. Further, the invention provides methods for preventing the recurrence of aseptic loosening of a joint replacement in patients that have been treated and have no disease activity by administering a liquid formulation.

The liquid formulation of the invention may be administered to a subject in need thereof to prevent, treat, manage or ameliorate a disorder associated with abnormal bone resorption or one or more symptoms thereof. The liquid formulations of the invention may also be administered in combination with one or more therapies (e.g., prophylactic or therapeutic agents) to a subject in need thereof to prevent, manage, treat or ameliorate a disorder associated with abnormal bone resorption or one or more symptoms thereof. Non-limiting examples of such therapies include, but are not limited to, sex hormones (e.g., estrogen), bisphosphonates (e.g., alendronate, etidronate, clodronate, ibandronate, pamidronate, risedronate, tiludronate, and zoledronate), calcitonin, and exercise programs. In a specific embodiment, the invention provides methods of preventing, managing, treating or ameliorating a disorder associated with abnormal bone resorption or one or more symptoms thereof, said method comprising administering to a subject in need thereof a dose of a prophylactically or therapeutically effective amount of a liquid formulation of the invention. In another embodiment, the invention provides methods of preventing, managing, treating or ameliorating a disorder associated with abnormal bone resorption or one or more symptoms thereof, said method comprising administering to a subject in need thereof a dose of a prophylactically or therapeutically effective amount of a liquid formulation of the invention and a dose prophylactically or therapeutically effective amount of one or more therapies (e.g., prophylactic or therapeutic agents) other than antibodies or antibody fragments that immunospecifically bind to integrin αᵥβ₃.

The liquid formulations of the invention may be used as first, second, third or fourth line of treatment for a disorder associated with abnormal bone resorption. The invention provides methods for managing, treating or ameliorating a disorder associated with abnormal bone resorption or one or more symptoms thereof in a subject refractory to conventional therapies for such a disease, said methods comprising administering to said subject a dose of a prophylactically or therapeutically effective amount of a liquid formulation of the invention. The invention also provides methods for managing, treating or ameliorating a disorder associated with abnormal bone resorption or one or more symptoms thereof in a subject refractory to existing single agent therapies for such a disease, said methods comprising administering to said subject a dose of a prophylactically or therapeutically effective amount of a liquid formulation of the invention and a dose prophylactically or therapeutically effective amount of one or more therapies (e.g., prophylactic or therapeutic agents) other than antibodies or fragments thereof that immunospecifically bind to integrin αᵥβ₃. The invention also provides alternative methods for the management or treatment of a disorder associated with abnormal bone resorption where conventional therapies have proven or may prove too toxic, i.e., results in unacceptable or unbearable side effects, for the subject being treated. Further, the invention provides methods for preventing the recurrence of a disorder associated with abnormal bone resorption in patients that have been treated and have no disease activity by administering a liquid formulation.

Disorders associated with abnormal bone resorption include, but are not limited to, parathyroid-related disorders (non-limiting examples are primary hyperparathyroidism, lithium therapy and familial hypocalciuric hypercalcemia); malignancy-related disorders (non-limiting examples are solid tumor with metastases, solid tumor with humoral mediation of hypercalcemia, and hematologic malignancies); vitamin D-related disorders (non-limiting examples are vitamin D intoxication, sarcoidosis and other granulomatous diseases, idiopathic hypercalcemia of infancy); and other diseases or disorders associated with high bone turnover (non-limiting examples are hyperthyroidism, immobilization, thiazide, and vitamin A intoxication).

5.5.2.1. Agents for Use in Regulating Bone Metabolism

The present invention provides methods of preventing, treating, managing or ameliorating diseases or disorders associated with abnormal bone metabolism or one or more symptoms thereof, said methods comprising administering to a subject in need thereof a liquid formulation of the invention and one or more therapies (e.g., prophylactic or therapeutic agents) other than antibodies or antibody fragments thereof that immunospecifically bind to integrin α_{V}β₃. Therapeutic or prophylactic agents include, but are not limited to, peptides, polypeptides, proteins, fusion proteins, nucleic acid molecules, small molecules, mimetic agents, synthetic drugs, inorganic molecules, and organic molecules. Any agent or therapy which is know to be useful, or which has been used or is currently being used to regulate bone metabolism can be used in combination with a liquid formulation of the invention in accordance with the invention described herein. Examples of such agents or therapies include, but are not limited to, phosphate, aluminum hydroxide, aluminum carbonate gels, magnesium, vitamin D, calcitriol, vitamin D₂ (ergocalciferol), vitamin D₃ (cholecalciferol), calcium, lithium, glucocorticoids, biphosphonates or a pharmaceutically acceptable salt or ester thereof (non-limiting examples are alendronate, clodronate, etidronate, ibandronate, pamidronate, risedronate, tiludronate, and zoledronate), calcitonin, plicamycin (mithramycin), gallium nitrate, estrogens, progestins, estrogen antagonists (e.g., tamoxifen), estrogen receptor modulators, androgen receptor modulators, cytotoxic or antiproliferative agents, matrix metalloproteinase inhibitors, inhibitors of epidermal-derived, fibroblast-derived, or platelet-derived growth factors, inhibitors of VEGF, antibodies to a growth factor or to a growth factor receptor, inhibitors of Flk-1/KDR, Flt-1, Tck/Tie-2, or Tie-1, cathepsin K inhibitors, inhibitors of osteoclast proton ATPase, inhibitors of urokinase plasminogen activator (u-PA), tumor-specific antibody-interleukin-2 fusion proteins, inhibitors of HMG-CoA reductase (non-limiting examples are prenylation inhibitors (non-limiting examples are Lovastatin, Pravastatin, Fluvastatin, Statin, Simvastatin, cerivastatin, lescol, lupitor, rosuvastatin and Atorvastatin), farnesyl transferase inhibitor, geranylgeranyl transferase inhibitor or dual farnesyl/geranylgeranyl transferase inhibitors), parathyroid hormone or parathyroid hormone fragments (a non-limiting example is exogenous PTH analogue, 1-34 PTH), growth hormones, molecules disclosed in U.S. Pat. Nos. 6,472,402 and 6,482,411, renal dialysis, surgery, or a combination thereof.

Therapies for disorder associated with aberrant bone metabolism and their dosages, routs of administration and recommended usage are known in the are and have been described in such literatures as the Physician's Desk Reference (56th ed., 2002 and 57th ed., 2003).

5.5.3. Cancer Treatment

The liquid formulations of the invention may be administered to a subject in need thereof to prevent, treat, manage or ameliorate a cancer or one or more symptoms thereof. The liquid formulations of the invention may also be administered in combination with one or more other therapies, preferably therapies useful for the prevention, management or treatment of cancer (including, but not limited to the prophylactic or therapeutic agents listed in Section 5.5.3.1 hereinbelow) to a subject in need thereof to prevent, treat, manage or ameliorate a cancer or one or more symptoms thereof. In a specific embodiment, the invention provides a method of preventing, treating, managing or ameliorating cancer or one or more symptoms thereof, said method comprising administering to a subject in need thereof a dose of a prophylactically or therapeutically effective amount of a liquid formulation of the invention. In another embodiment, the invention provides a method of preventing, treating or ameliorating cancer or one or more symptoms thereof, said method comprising administering to a subject in need thereof a dose of a prophylactically or therapeutically effective amount of a liquid formulation of the invention and a dose of a prophylactically or therapeutically effective amount of one or more therapies (e.g., prophylactic or therapeutic agents other than antibodies or antibody fragments that immunospecifically bind to integrin α_{V}β₃.

The liquid formulations of the invention may be used as a first, second, third or fourth line cancer treatment. The invention provides methods for treating or ameliorating one or more symptoms of a cancer in a subject refractory to conventional therapies for such a cancer, said methods comprising administering to said subject a dose of a prophylactically or therapeutically effective amount of a liquid formulation of the invention. A cancer may be determined to be refractory to a therapy means when at least some significant portion of the cancer cells are not killed or their cell division arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased.

The invention provides methods for managing, treating or ameliorating cancer or one or more symptoms thereof in a subject refractory to existing single agent therapies for such a cancer, said methods comprising administering to said subject a dose of a prophylactically or therapeutically effective amount of a liquid formulation of the invention and a dose of a prophylactically or therapeutically effective amount of one or more therapies (e.g., prophylactic or therapeutic agents) other than antibodies or antibody fragments that immunospecifically bind to integrin α_{V}β₃. The invention also provides methods for managing, treating or ameliorating cancer by administering a liquid formulation of the invention in combination with any other treatment (e.g., radiation therapy, chemotherapy or surgery) to patients who have proven refractory to other treatments but are no longer on these treatments. The invention also provides methods for the management or treatment of a patient having cancer and immunosuppressed by reason of having previously undergone other cancer therapies. The invention also provides alternative methods for the management, treatment or amelioration of cancer or one or more symptoms thereof, where chemotherapy, radiation therapy, hormonal therapy, and/or biological therapy/immunotherapy has proven or may prove too toxic, i.e., results in unacceptable or unbearable side effects, for the subject being treated. Further, the invention provides methods for preventing the recurrence of cancer in patients that have been treated and have no disease activity by administering a liquid formulation of the invention.

Cancers that can be treated by the methods encompassed by the invention include, but are not limited to, neoplasms, tumors, metastases, or any disease or disorder characterized by uncontrolled cell growth. The cancer may be a primary or metastatic cancer. The cancer may or may not express integrin α_{V}β₃. In a preferred embodiment, the cancer that is being managed, treated or ameliorated in accordance with the methods of the invention is a cancer expressing integrin α_{V}β₃ that has metastasized to the bone. Specific examples of cancers that can be treated by the methods encompassed by the invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone cancer and connective tissue sarcomas such as but not limited to bone sarcoma, myeloma bone disease, osteosarcoma, chondrosarcoma, Ewing's sarcoma, Paget's disease of bone, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease), and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America). It is also contemplated that cancers caused by aberrations in apoptosis can also be treated by the methods and compositions of the invention. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes.

In a preferred embodiment, the cancer that is being prevented, managed, treated or ameliorated in accordance with the method of the invention is prostate cancer, breast cancer, bone cancer, melanoma, lung cancer and ovarian cancer. In another embodiment, the cancer that is being prevented, managed, treated or ameliorated in accordance with the methods of the invention are metastatic tumors including, but not limited to, tumors that have or may metastasize to the bone (non-limiting examples are prostate, breast and lung cancers that have metastasized or have the potential to metastasize to the bone), tumors that have or may metastasize to the lung, tumors that have or may metastasize to the brain, and tumors that have or may metastasize to other organs or tissues of a subject.

5.5.3.1. Anti-Cancer Therapies

The present invention provides methods of preventing, managing, treating or ameliorating cancer or one or more symptoms thereof, said methods comprising administering to a subject in need thereof a liquid formulation of the invention and one or more therapies (e.g. prophylactic or therapeutic agents) other than antibodies or antibody fragments that immunospecifically bind to integrin αᵥβ₃. Therapeutic or prophylactic agents include, but are not limited to, peptides, polypeptides, proteins, fusion proteins, nucleic acid molecules, small molecules, mimetic agents, synthetic drugs, inorganic molecules, and organic molecules. Any agent or therapy (e.g., chemotherapies, radiation therapies, hormonal therapies, and/or biological therapies/immunotherapies) which is known to be useful, or which has been used or is currently being used for the prevention, treatment, management or amelioration of cancer or one or more symptoms thereof can be used in combination with a liquid formulation of the invention in accordance with the invention described herein. Examples of such agents (i.e., anti-cancer agents) include, but are not limited to, angiogenesis inhibitors, topoisomerase inhibitors and immunomodulatory agents (such as chemotherapeutic agents and non-therapeutic immunomodulatory agents, including but not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., MEDI-507 (MedImmune, Inc., International Publication Nos. WO 02/098370 and WO 02/069904), anti-CD11a antibodies (e.g., Xanelim (Genentech)), and anti-B7 antibodies (e.g., IDEC-114) (IDEC)); anti-cytokine receptor antibodies (e.g., anti-IEFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies)); CTLA4-immunoglobulin; LFA-3TIP (Biogen, International Publication No. WO 93/08656 and U.S. Pat. No. 6,162,432); soluble cytokine receptors (e.g., the extracellular domain of a TNF-α receptor or a fragment thereof, the extracellular domain of an IL-11 receptor or a fragment thereof, and the extracellular domain of an IL-6 receptor or a fragment thereof); cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF); and anti-cytokine antibodies (e.g., anti-IL-2 antibodies, anti-IL-4 antibodies, anti-IL-6 antibodies, anti-IL-10 antibodies, anti-IL-12 antibodies, anti-IL-15 antibodies, anti-TNF-α antibodies, and anti-IFN-γ antibodies), and antibodies that immunospecifically bind to tumor-associated antigens (e.g., Herceptin®).

Angiogenesis inhibitors (i.e., anti-angiogenic agents) include, but are not limited to, angiostatin (plasminogen fragment); antiangiogenic antithrombin III; angiozyme; ABT-627; Bay 12-9566; Benefin; Bevacizumab; BMS-275291; cartilage-derived inhibitor (CDI); CAI; CD59 complement fragment; CEP-7055; Col 3; combretastatin A-4; endostatin (collagen XVIII fragment); fibronectin fragment; Gro-beta; Halofuginone; Heparinases; Heparin hexasaccharide fragment; HMV833; human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-methoxyestradiol; MMI 270 (CGS 27023A); MoAb IMC-1C11; Neovastat; NM-3; Panzem; PI-88; Placental ribonuclease inhibitor; plasminogen activator inhibitor; platelet factor-4 (PF4); Prinomastat; Prolactin 16 kD fragment; Proliferin-related protein (PRP); PTK 787/ZK 222594; retinoids; solimastat; squalamine; SS 3304; SU 5416; SU6668; SU11248; tetrahydrocortisol-S; tetrathiomolybdate; thalidomide; thrombospondin-1 (TSP-1); TNP-470; transforming growth factor-beta; vasculostatin; vasostatin (calreticulin fragment); ZD6126; ZD 6474; HMG-CoA reductase inhibitor (3-Hydroxy-3-Methyl-Glutaryl Coenzyme A reductase inhibitor) include, but not limited to, Lovastatin, Pravastatin, Fluvastatin, Statin, Simvastatin, and Atorvastatin; farnesyl transferase inhibitors (FTI); and bisphosphonates include, but are not limited to, alendronate, clodronate, etidronate, ibandronate, pamidronate, risedronate, tiludronate, and zoledronate. In a specific embodiment, anti-angiogenic agents do not include antibodies or fragments thereof that immunospecifically bind to integrin α_{V}β₃.

Specific examples of anti-cancer agents which can be used in accordance with the methods of the invention include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vitaxin; vorozole; zanoterone; zeniplatin; zilascorb; and zinostatin stimalamer.

The invention also encompasses administration of a liquid formulation of the invention in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radioactive source is placed inside the body close to cancer cells or a tumor mass.

In specific embodiments, patients with breast cancer are administered a prophylactically or therapeutically effective amount of a liquid formulation of the invention in combination with the administration of a prophylactically or therapeutically effective amount of one or more other agents useful for breast cancer therapy including but not limited to: doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), Herceptin®, tamoxifen, the combination of tamoxifen and cytotoxic chemotherapy. In certain embodiments, patients with metastatic breast cancer are administered a prophylactically or therapeutically effective amount of one or more liquid formulations of the invention in combination with the administration of an effective amount of taxanes such as docetaxel and paclitaxel. In other embodiments, a prophylactically or therapeutically effective amount of a liquid formulation of the invention is administered in combination with the administration of a prophylactically or therapeutically effective amount of taxanes plus standard doxorubicin and cyclophosphamide for adjuvant treatment of node-positive, localized breast cancer.

In specific embodiments, patients with prostate cancer are administered a prophylactically or therapeutically effective amount of a liquid formulation of the invention in combination with the administration of a prophylactically or therapeutically effective amount of one or more other agents useful for prostate cancer therapy including but not limited to: external-beam radiation therapy, interstitial implantation of radioisotopes (i.e., I¹²⁵, palladium, Iridium), leuprolide or other LHRH agonists, non-steroidal antiandrogens (flutamide, nilutamide, bicalutamide), steroidal antiandrogens (cyproterone acetate), the combination of leuprolide and flutamide, estrogens such as DES, chlorotrianisene, ethinyl estradiol, conjugated estrogens U.S.P., DES-diphosphate, radioisotopes, such as strontium-89, the combination of external-beam radiation therapy and strontium-89, second-line hormonal therapies such as aminoglutethimide, hydrocortisone, flutamide withdrawal, progesterone, and ketoconazole, low-dose prednisone, or other chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel. In specific embodiments, patients with ovarian cancer are administered a prophylactically or therapeutically effective amount of a liquid formulation of the invention in combination with a prophylactically or therapeutically effective amount of one or more other agents useful for ovarian cancer therapy including but not limited to: intraperitoneal radiation therapy, such as P³² therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. It is contemplated that a prophylactically or therapeutically effective amount of a liquid formulation of the invention is administered in combination with the administration Taxol for patients with platinum-refractory disease. Included is the treatment of patients with refractory ovarian cancer including administration of: ifosfamide in patients with disease that is platinum-refractory, hexamethylmelamine (HMM) as salvage chemotherapy after failure of cisplatin-based combination regimens, and tamoxifen in patients with detectable levels of cytoplasmic estrogen receptor on their tumors. In specific embodiments, patients with bone sarcomas are administered a prophylactically or therapeutically effective amount of a liquid formulation of the invention in combination with a prophylactically or therapeutically effective amount of one or more other agents useful for bone sarcoma therapy including but not limited to: doxorubicin, ifosfamide, cisplatin, high-dose methotrexate, cyclophosphamide, etoposide, vincristine, dactinomycin, and surgery.

In specific embodiments, patients with tumor metastatic to bone are administered a prophylactically or therapeutically effective amount of a liquid formulation of the invention in combination with a prophylactically or therapeutically effective amount of one or more other agents useful for bone metastatic tumor therapy including but not limited to: agents or therapies used in treatment of underlying malignancy (non-limiting examples are hormone inhibitors for prostate or breast cancer metastasized to bone and surgery), radiotherapy (non-limiting examples are strontium 89 and samarium 153, which are bone-seeking radionuclides that can exert antitumor effects and relieve symptoms), and bisphosphonates.

In more particular embodiments, the present invention also comprises the administration of a liquid formulation of the invention in combination with the administration of one or more therapies such as, but not limited to anti-cancer agents such as those disclosed in Table 3, preferably for the prevention, management, treatment or amelioration of breast, bone, ovary, and prostate cancers as described above.

TABLE 3

| Therapeutic | | | |
|---|---|---|---|
| Agent | Dose/Administration/Formulation | | |
| doxorubicin | Intravenous | 60-75 mg/m² on Day 1 | 21 day intervals |
| hydrochloride | | | |
| (Adriamycin | | | |
| RDF ® and | | | |
| Adriamycin | | | |
| PFS ®) | | | |
| epirubicin | Intravenous | 100-120 mg/m² on Day | 3-4 week cycles |
| hydrochloride | | 1 of each cycle or | |
| (Ellence ™) | | divided equally and | |
| | | given on Days 1-8 of | |
| | | the cycle | |
| fluorousacil | Intravenous | How supplied: | |
| | | 5 mL and 10 mL vials | |
| | | (containing 250 and 500 mg | |
| | | flourouracil | |
| | | respectively) | |
| docetaxel | Intravenous | 60-100 mg/m² over 1 | Once every 3 weeks |
| (Taxotere ®) | | hour | |
| paclitaxel | Intravenous | 175 mg/m² over 3 hours | Every 3 weeks for |
| (Taxol ®) | | | 4 courses (administered |
| | | | sequentially to |
| | | | doxorubicin-containing |
| | | | combination |
| | | | chemotherapy) |
| tamoxifen | Oral | 20-40 mg | Daily |
| citrate | (tablet) | Dosages greater than 20 mg | |
| (Nolvadex ®) | | should be given in | |
| | | divided doses (morning | |
| | | and evening) | |
| leucovorin | Intravenous or | How supplied: | Dosage is unclear from |
| calcium for | intramuscular | 350 mg vial | text. PDR 3610 |
| injection | injection | | |
| luprolide | Single | 1 mg (0.2 mL or 20 unit | Once a day |
| acetate | subcutaneous | mark) | |
| (Lupron ®) | injection | | |
| flutamide | Oral (capsule) | 250 mg | 3 times a day at 8 hour |
| (Eulexin ®) | | (capsules contain 125 mg | intervals (total daily |
| | | flutamide each) | dosage 750 mg) |
| nilutamide | Oral | 300 mg or 150 mg | 300 mg once a day for 30 |
| (Nilandron ®) | (tablet) | (tablets contain 50 or | days followed by 150 mg |
| | | 150 mg nilutamide | once a day |
| | | each) | |
| bicalutamide | Oral | 50 mg | Once a day |
| (Casodex ®) | (tablet) | (tablets contain 50 mg | |
| | | bicalutamide each) | |
| progesterone | Injection | USP in sesame oil 50 mg/mL | |
| ketoconazole | Cream | 2% cream applied once | |
| (Nizoral ®) | | or twice daily | |
| | | depending on symptoms | |
| prednisone | Oral | Initial dosage may vary | |
| | (tablet) | from 5 mg to 60 mg per | |
| | | day depending on the | |
| | | specific disease entity | |
| | | being treated. | |
| estramustine | Oral | 14 mg/kg of body | Daily given in 3 or 4 |
| phosphate | (capsule) | weight (i.e. one 140 mg | divided doses |
| sodium | | capsule for each 10 kg | |
| (Emcyt ®) | | or 22 lb of body weight) | |
| etoposide or | Intravenous | 5 mL of 20 mg/mL | |
| VP-16 | | solution (100 mg) | |
| dacarbazine | Intravenous | 2-4.5 mg/kg | Once a day for 10 days. |
| (DTIC-Dome ®) | | | May be repeated at 4 week |
| | | | intervals |
| polifeprosan 20 | wafer placed in | 8 wafers, each | |
| with carmustine | resection | containing 7.7 mg of | |
| implant | cavity | carmustine, for a total | |
| (BCNU) | | of 61.6 mg, if size and | |
| (nitrosourea) | | shape of resection | |
| (Gliadel ®) | | cavity allows | |
| cisplatin | Injection | [n/a in PDR 861] | |
| | | How supplied: | |
| | | solution of 1 mg/mL in | |
| | | multi-dose vials of | |
| | | 50 mL and 100 mL | |
| mitomycin | Injection | supplied in 5 mg and 20 mg | |
| | | vials (containing 5 mg | |
| | | and 20 mg | |
| | | mitomycin) | |
| gemcitabine | Intravenous | For NSCLC-2 | 4 week schedule- |
| HCl (Gemzar ®) | | schedules have been | Days 1, 8 and 15 of each |
| | | investigated and the | 28-day cycle. Cisplatin |
| | | optimum schedule has | intravenously at 100 mg/m² |
| | | not been determined | on day 1 after the |
| | | 4 week schedule- | infusion of Gemzar. |
| | | administration | 3 week schedule- |
| | | intravenously at 1000 mg/m² | Days 1 and 8 of each 21 |
| | | over 30 minutes | day cycle. Cisplatin at |
| | | on 3 week schedule- | dosage of 100 mg/m² |
| | | Gemzar administered | administered |
| | | intravenously at 1250 mg/m² | intravenously after |
| | | over 30 minutes | administration of Gemzar |
| | | | on day 1. |
| carboplatin | Intravenous | Single agent therapy: | Every 4 weeks |
| (Paraplatin ®) | | 360 mg/m² I.V. on day 1 | |
| | | (infusion lasting 15 | |
| | | minutes or longer) | |
| | | Other dosage | |
| | | calculations: | |
| | | Combination therapy | |
| | | with cyclophosphamide, | |
| | | Dose adjustment | |
| | | recommendations, | |
| | | Formula dosing, etc. | |
| ifosamide | Intravenous | 1.2 g/m² daily | 5 consecutive days |
| (Ifex ®) | | | Repeat every 3 weeks or |
| | | | after recovery from |
| | | | hematologic toxicity |
| topotecan | Intravenous | 1.5 mg/m² by | 5 consecutive days, |
| hydrochloride | | intravenous infusion | starting on day 1 of 21 day |
| (Hycamtin ®) | | over 30 minutes daily | course |
| Bisphosphonates | | | |
| Pamidronate | Intravenous | 60 mg or 90 mg single | |
| | | infusion over 4-24 | |
| | | hours to correct | |
| | | hypercalcemia in cancer | |
| | | patients | |
| Alendronate | Oral | 5 mg/d daily for 2 years | |
| | | and then 10 mg/d for 9 | |
| | | month to prevent or | |
| | | control bone resorption. | |
| Risedronate | Oral, take with | 5.0 mg to prevent or | |
| | 6-8 oz water. | control bone resorption. | |
| Lovastatin | Oral | 10-80 mg/day in single | |
| (Mevacor ™) | | or two divided dose. | |

Cancer therapies and their dosages, routes of administration and recommended usage are known in the art and have been described in such literature as the Physician's Desk Reference (56^{th} ed., 2002 and 57^{th} ed., 2003).

5.5.4. Inflammatory Disorder Treatment

The liquid formulations of the invention may be administered to a subject in need thereof to prevent, manage, treat or ameliorate an inflammatory disorder or one or more symptoms thereof. The liquid formulations of the invention may also be administered in combination with one or more other therapies, preferably therapies useful for the prevention, management, treatment or amelioration of an inflammatory disorder (including, but not limited to the prophylactic or therapeutic agents listed in Section 5.5.4.1 hereinbelow) to a subject in need thereof to prevent, manage, treat or ameliorate an inflammatory disorder or one or more symptoms thereof. In a specific embodiment, the invention provides a method of preventing, managing, treating or ameliorating an inflammatory disorder or one or more symptoms thereof, said method comprising administering to a subject in need thereof a dose of a prophylactically or therapeutically effective amount of a liquid formulation of the invention. In another embodiment, the invention provides a method of preventing, managing, treating or ameliorating an inflammatory disorder or one or more symptoms thereof, said method comprising administering to a subject in need thereof a dose of a prophylactically or therapeutically effective amount of a liquid formulation of the invention and a dose of a prophylactically or therapeutically effective amount of one or more therapies (e.g., prophylactic or therapeutic agents) other than antibodies or antibody fragments that immunospecifically bind to integrin α_{V}β₃.

The invention provides methods for managing, treating or ameliorating one or more symptoms of an inflammatory disorder in a subject refractory to conventional therapies (e.g., methotrexate and a TNF-α antagonist (e.g., REMICADE™ or ENBREL™)) for such an inflammatory disorder, said methods comprising administering to said subject a dose of a prophylactically or therapeutically effective amount of a liquid formulation of the invention. The invention also provides methods for managing, treating or ameliorating one or more symptoms of an inflammatory disorder in a subject refractory to existing single agent therapies for such an inflammatory disorder, said methods comprising administering to said subject a dose of a prophylactically or therapeutically effective amount of a liquid formulation of the invention and a dose of a prophylactically or therapeutically effective amount of one or more therapies (e.g., prophylactic or therapeutic agents) other than antibodies or antibody fragments that immunospecifically bind to integrin α_{V}β₃. The invention also provides methods for managing or treating an inflammatory disorder by administering a liquid formulation of the invention in combination with any other treatment to patients who have proven refractory to other treatments but are no longer on these treatments. The invention also provides alternative methods for the treatment of an inflammatory disorder where another therapy has proven or may prove too toxic, i.e., results in unacceptable or unbearable side effects, for the subject being treated. For example, the liquid formulations of the invention may be administered to a subject, wherein the subject is refractory to a TNF antagonist or methotrexate. Further, the invention provides methods for preventing the recurrence of an inflammatory disorder in patients that have been treated and have no disease activity by administering a liquid formulation of the invention.

Inflammatory disorders that can be treated by the methods encompassed by the invention include, but are not limited to, asthma, encephilitis, inflammatory bowel disease, chronic obstructive pulmonary disease (COPD), allergic disorders, septic shock, pulmonary fibrosis, undifferentiated spondyloarthropathy, undifferentiated arthropathy, arthritis, osteoarthritis, spondyloarthropathies (e.g., psoriatic arthritis, ankylosing spondylitis, Reiter's Syndrome (reactive arthritis), inflammatory osteolysis, Wilson's disease and chronic inflammation resulting from chronic viral or bacteria infections. As described herein in Section 3.1, some autoimmune disorders are associated with an inflammatory condition.

Anti-inflammatory therapies and their dosages, routes of administration and recommended usage are known in the art and have been described in such literature as the Physician's Desk Reference (56th ed., 2002 and 57th ed., 2003).

5.5.4.1. Anti-Inflammatory Therapies

The present invention provides methods of preventing, managing, treating or ameliorating an inflammatory disorder or one or more symptoms thereof, said methods comprising administering to a subject in need thereof a liquid formulation of the invention and one or more therapies (e.g., prophylactic or therapeutic agents other than antibodies or antibody fragments that immunospecifically bind to integrin αᵥβ₃. Any agent or therapy which is known to be useful, or which has been used or is currently being used for the prevention, management, treatment or amelioration of an inflammatory disorder or one or more symptoms thereof can be used in combination with a liquid formulation of the invention in accordance with the invention described herein. Examples of such agents include, but are not limited to, immunomodulatory agents, an anti-angiogenic agents, anti-inflammatory agents and TNF-α antagonists.

Specific examples of immunomodulatory agents which can be administered in combination with a liquid formulation of the invention to a subject with an inflammatory disorder include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., MEDI-507 (MedImmune, Inc., International Publication Nos. WO 02/098370 and WO 02/069904), anti-CD11a antibodies (e.g., Xanelim (Genentech)), and anti-B7 antibodies (e.g., IDEC-114) (IDEC)); anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies)); CTLA4-immunoglobulin; LFA-3TIP (Biogen, International Publication No. WO 93/08656 and U.S. Pat. No. 6,162,432); soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof); cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF); and anti-cytokine antibodies (e.g., anti-IL-2 antibodies, anti-IL-4 antibodies, anti-IL-6 antibodies, anti-IL-10 antibodies, anti-IL-12 antibodies, anti-IL-15 antibodies, anti-TNF-α antibodies, and anti-IFN-γ antibodies).

Non-limiting examples of anti-angiogenic agents which can be administered in combination with a liquid formulation of the invention to a subject with an inflammatory disorder include endostatin, angiostatin, apomigren, anti-angiogenic antithrombin III, the 29 kDa N-terminal and a 40 kDa C-terminal proteolytic fragments of fibronectin, a uPA receptor antagonist, the 16 kDa proteolytic fragment of prolactin, the 7.8 kDa proteolytic fragment of platelet factor-4, the anti-angiogenic 24 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, RGD and NGR containing peptides, the small anti-angiogenic peptides of laminin, fibronectin, procollagen and EGF, acid fibroblast growth factor (aFGF) antagonists, basic fibroblast growth factor (bFGF) antagonists, vascular endothelial growth factor (VEGF) antagonists, and VEGF receptor (VEGFR) antagonists (e.g., anti-VEGFR antibodies).

Non-limiting examples of TNF-α antagonists which can be administered in combination with a liquid formulation of the invention to a subject with an inflammatory disorder include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that blocks, reduces, inhibits or neutralizes the function, activity and/or expression of TNF-α. In various embodiments, a TNF-α antagonist reduces the function, activity and/or expression of TNF-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS). Examples of antibodies that immunospecifically bind to TNF-α include, but are not limited to, infliximab (REMICADE™; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379). The present invention also encompasses the use of antibodies that immunospecifically bind to TNF-α disclosed in the following U.S. patents in the compositions and methods of the invention: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety. Examples of soluble TNF-α receptors include, but are not limited to, sTNF-R1 (Amgen), etanercept (ENBREL™; Immunex) and its rat homolog RENBREL™, soluble inhibitors of TNF-α derived from TNFrI, TNFrII (Kohno et al., 1990, Proc. Natl. Acad. Sci. USA 87:8331-8335), and TNF-α Inh (Seckinger et al, 1990, Proc. Natl. Acad. Sci. USA 87:5188-5192).

Other TNF-α antagonists encompassed by the invention include, but are not limited to, IL-10, which is known to block TNF-α production via interferon γ-activated macrophages (Oswald et al. 1992, Proc. Natl. Acad. Sci. USA 89:8676-8680), TNFR-IgG (Ashkenazi et al., 1991, Proc. Natl. Acad. Sci. USA 88:10535-10539), the murine product TBP-1 (Serono/Yeda), the vaccine CytoTAb (Protherics), antisense molecule104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach.

Non-limiting examples of anti-inflammatory agents which can be administered in combination with a liquid formulation of the invention to a subject with an inflammatory disorder include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

In specific embodiments, patients with osteoarthritis are administered a prophylactically or therapeutically effective amount of a liquid formulation of the invention in combination with other agents or therapies useful for osteoarthritis prevention, treatment, management or amelioration including but not limited to: analgesics (non-limiting examples are acetaminophen, in a dose up to 4000 mg/d; phenacetin; and tramadol, in a daily dose in the range of 200 to 300 mg); NSAIDs (non-limiting examples include but not limited to, aspirin, diflunisal, diclofenac, etodolac, fenamates, fenoprofen, flurbiprofen, ibuprofen, indomethacin, ketoprofen, methylsalicylate, nebumetone, naproxin, oxaprazin, phenylbutazone, piroxicam, sulindac, and tolmetin. Low dose NSAIDs are preferred, e.g., ibuprofen at 1200 mg/d, naproxen at 500 mg/d. A gastroprotective agent, e.g., misoprostol, famotidine or omeprazole, is preferred to use concurrently with a NSAID); nonacetylated salicylates including but not limited to salsalate; cyclooxygenase (Cox)-2-specific inhibitors (CSIs), including but not limited to, celecoxib and rofecoxib; intra- or periarticular injection of a depot glucocorticoid preparation; intra-articular injection of hyaluronic acid; capsaicin cream; copious irrigation of the osteroarthritis knee to flush out fibrin, cartilage shards and other debris; and joint replacement surgery. The liquid formulations of the invention can also be used in combination with other nonpharmacologic measures in prevention, treatment, management and amelioration of osteoarthritis including but not limited to: reduction of joint loading (non-limiting examples are correction of poor posture, support for excessive lumbar lordosis, avoid excessive loading of the involved joint, avoid prolonged standing, kneeling and squatting); application of heat to the affected joint; aerobic exercise and other physical therapies.

In specific embodiments, patients with rheumatoid arthritis are administered a prophylactically or therapeutically effective amount of a liquid formulation of the invention in combination with other agents or therapies useful in prevention, treatment, management and amelioration of rheumatoid arthritis including but not limited to: NSAIDs (non-limiting examples include but not limited to, aspirin, diflunisal, diclofenac, etodolac, fenamates, fenoprofen, flurbiprofen, ibuprofen, indomethacin, ketoprofen, methylsalicylate, nebumetone, naproxin, oxaprazin, phenylbutazone, piroxicam, sulindac, and tolmetin); analgesics (non-limiting examples are acetaminophen, phenacetin and tramadol); CSIs including but not limited to, celecoxib and rofecoxib; glucocorticoids (preferably low-dose oral glucocorticoids, e.g., <7.5 mg/d prednisone, or monthly pulses with high-dose glucocorticoids, or intraarticular glucocorticoids); disease-modifying antirheumatic drugs (DMARDs) including but not limited to, methotrexate (preferably given intermittent low dose, e.g., 7.5-30 mg once weekly), gold compounds (e.g., gold salts), D-penicillamine, the antimalarials (e.g., chloroquine), and sulfasalazine; TNF-α neutralizing agents including but not limited to, etanercept and infliximab; immunosuppressive and cytotoxic agents (examples include but not limited to, azathioprine, leflunomide, cyclosporine, and cyclophosphamide), and surgery (examples include but not limited to, arthroplasties, total joint replacement, reconstructive hand surgery, open or arthroscopic synovectomy, and early tenosynovectomy of the wrist). The liquid formulations of the invention may also be used in combination with other measures in prevention, treatment, management and amelioration of the rheumatoid arthritis including but not limited to: rest, splinting to reduce unwanted motion of inflamed joint, exercise, used of a variety of orthotic and assistive devices, and other physical therapies. The liquid formulations of the invention may also be used in combination with some nontraditional approaches in prevention, treatment, management and amelioration of rheumatoid arthritis including but not limited to, diets (e.g., substituting omega-3 fatty acids such as eicosapentaenoic acid found in certain fish oils for dietary omega-6 essential fatty acids found in meat), vaccines, hormones and topical preparations.

In specific embodiments, patients with chronic obstructive pulmonary disease (COPD) are administered a prophylactically or therapeutically effective amount of a liquid formulation of the invention in combination with other agents or therapies useful in prevention, treatment, management and amelioration of COPD including but not limited to: bronchodilators including but not limited to, short- and long-acting β₂-adrenergic agonists (examples of short-acting β₂ agonist include but not limited to, albuterol, pirbuterol, terbutaline, and metaproterenol; examples of long-acting β₂ agonist include but not limited to, oral sustained-release albuterol and inhaled salmeterol), anticholinergics (examples include but not limited to ipratropium bromide), and theophylline and its derivatives (therapeutic range for theophylline is preferably 10-20 μg/mL); glucocorticoids; exogenous α₁AT (e.g., α₁AT derived from pooled human plasma administered intravenously in a weekly dose of 60 mg/kg); oxygen; lung transplantation; lung volume reduction surgery; endotracheal intubation, ventilation support; yearly influenza vaccine and pneumococcal vaccination with 23-valent polysaccharide; exercise; and smoking cessation.

In specific embodiments, patients with pulmonary fibrosis are administered a prophylactically or therapeutically effective amount of a liquid formulation of the invention in combination with an effective amount of one or more other agents useful for pulmonary fibrosis therapy including but not limited to: oxygen; corticosteroids (a non-limiting example is to administer daily prednisone beginning at 1-1.5 mg/kg/d (up to 100 mg/d) for six weeks and tapering slowly over 3-6 months to a minimum maintenance dose of 0.25 mg/kg/d); cytotoxic drugs (non-limiting examples are cyclophosphamide at 100-120 mg orally once daily, and azathioprine at 3 mg/kg up to 200 mg orally once daily); bronchodilators (non-limiting examples are short- and long-acting β₂-adrenergic agonists, anticholinergics, and theophylline and its derivatives); and antihistamines (non-limiting examples are diphenhydramine and doxylamine).

In specific embodiments, patients with asthma are administered a prophylactically or therapeutically effective amount of a liquid formulation of the invention in combination with an effective amount of one or more other agents useful for asthma therapy including but not limited to: adrenergic stimulants (examples include but not limited to, catecholamines, e.g., epinephrine, isoproterenol, and isoetharine; resorcinols, e.g., metaproterenol, terbutaline, and fenoterol; and saligenins, e.g., salbutamol. Inhalation is the preferred route of administration for adrenergic stimulants); methylxanthines including but not limited to theophylline and its various salts; anticholinergics including but not limited to, atropine sulfate, atropine methylnitrate, and ipratropium bromide; glucocorticoids (examples including but not limited to systemic or oral steroids, and inhaled glucocorticoids); mast cell stabilizing agents (examples include but not limited to, cromolyn sodium and nedocromil sodium); leukotriene modifiers (examples include but not limited to, Zileuton, zafirlukast and montelukast); immunosuppressant agents (examples include but not limited to, methotrexate and gold salts); and mucolytic agents (examples include but not limited to acetylcysteine).

In specific embodiments, patients with allergy are administered a prophylactically or therapeutically effective amount of a liquid formulation of the invention in combination with an effective amount of one or more other agents useful for allergy therapy including but not limited to: cromolyn; antimmediator drugs (examples include but not limited to antihistamines, see Table 4); sympathomimetic drugs (examples include but not limited to α-adrenergic and β-adrenergic drugs); theophylline and its derivatives; glucocorticoids; and immunotherapy (examples include but not limited to, repeated long-term injection of allergen, short course desensitization, and venom immunotherapy).

TABLE 4
H₁ Antihistamines

| Chemical class and | |
|---|---|
| representative drugs | Usual daily dosage |
| Ethanolamine | |
| Diphehydramine | 25-50 mg every 4-6 hours |
| Clemastine | 0.34-2.68 mg every 12 hours |
| Ethylenediamine | |
| Tripelennamine | 25-50 mg every 4-6 hours |
| Alkylamine | |
| Brompheniramine | 4 mg every 4-6 hours; or 8-12 mg of SR |
| | form every 8-12 hour |
| Chlorpheniramine | 4 mg every 4-6 hours; or 8-12 mg of SR |
| | form every 8-12 hour |
| Triprolidine (1.25 mg/5 ml) | 2.5 mg every 4-6 hours |
| Phenothiazine | |
| Promethazine | 25 mg at bedtime |
| Piperazine | |
| Hydroxyzine | 25 mg every 6-8 hours |
| Piperidines | |
| Astemizole (nonsedating) | 10 mg/d |
| Azatadine | 1-2 mg every 12 hours |
| Cetirzine | 10 mg/d |
| Cyproheptadine | 4 mg every 6-8 hour |
| Fexofenadine (nonsedating) | 60 mg every 12 hours |
| Loratidine (nonsedating) | 10 mg every 24 hours |

5.5.5. Autoimmune Disorder Treatment

The liquid formulations of the invention may be administered to a subject in need thereof to prevent, manage, treat or ameliorate an autoimmune disorder or one or more symptoms thereof. The liquid formulations of the invention may also be administered in combination with one or more other therapies, preferably therapies useful for the prevention, management or treatment of an autoimmune disorder (including, but not limited to the prophylactic or therapeutic agents listed in Section 5.5.5.1 hereinbelow) to a subject in need thereof to prevent, manage, treat or ameliorate an autoimmune disorder or one or more symptoms thereof. In a specific embodiment, the invention provides a method of preventing, managing, treating or ameliorating an autoimmune disorder or one or more symptoms thereof, said method comprising administering to a subject in need thereof a dose of a prophylactically or therapeutically effective amount of a liquid formulation of the invention. In another embodiment, the invention provides a method of preventing, managing, treating or ameliorating an autoimmune disorder or one or more symptoms thereof, said method comprising administering to a subject in need thereof a dose of a prophylactically or therapeutically effective amount of a liquid formulation of the invention and a dose of a prophylactically or therapeutically effective amount of one or more therapies (e.g., prophylactic or therapeutic agents) other than antibodies or antibody fragments that immunospecifically bind to integrin α_{V}β₃.

The invention provides methods for managing, treating or ameliorating an autoimmune disorder or one or more symptoms thereof in a subject refractory to conventional therapies for such an autoimmune disorder, said methods comprising administering to said subject a dose of a prophylactically or therapeutically effective amount of a liquid formulation of the invention. The invention also provides methods for managing, treating or ameliorating an autoimmune disorder or one or more symptoms thereof in a subject refractory to existing single agent therapies for such an autoimmune disorder, said methods comprising administering to said subject a dose of a prophylactically or therapeutically effective amount of a liquid formulation of the invention and a dose of a prophylactically or therapeutically effective amount of one or more therapies (e.g., prophylactic or therapeutic agents) other than antibodies or antibody fragments that immunospecifically bind to integrin α_{V}β₃. The invention also provides methods for managing, treating or ameliorating an autoimmune disorder or one or more symptoms thereof by administering a liquid formulation of the invention in combination with any other treatment to patients who have proven refractory to other treatments but are no longer on these treatments. The invention also provides alternative methods for the management or treatment of an autoimmune disorder where another therapy has proven or may prove too toxic, i.e., results in unacceptable or unbearable side effects, for the subject being treated. Particularly, the invention provides alternative methods for the management or treatment of an autoimmune disorder where the patient is refractory to other therapies. Further, the invention provides methods for preventing the recurrence of an autoimmune disorder in patients that have been treated and have no disease activity by administering a liquid formulation of the invention.

In autoimmune disorders, the immune system triggers an immune response when there are no foreign substances to fight and the body's normally protective immune system causes damage to its own tissues by mistakenly attacking self. There are many different autoimmune disorders which affect the body in different ways. For example, the brain is affected in individuals with multiple sclerosis, the gut is affected in individuals with Crohn's disease, and the synovium, bone and cartilage of various joints are affected in individuals with rheumatoid arthritis. As autoimmune disorders progress destruction of one or more types of body tissues, abnormal growth of an organ, or changes in organ function may result. The autoimmune disorder may affect only one organ or tissue type or may affect multiple organs and tissues. Organs and tissues commonly affected by autoimmune disorders include red blood cells, blood vessels, connective tissues, endocrine glands (e.g., the thyroid or pancreas), muscles, joints, and skin. Examples of autoimmune disorders that can be treated by the methods of the invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Ménière's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteritis/giant cell arteritis, ulcerative colitis, uveitis, vasculitides such as dermatitis herpetiformis vasculitis, vitiligo, and Wegener's granulomatosis.

Autoimmune therapies and their dosages, routes of administration and recommended usage are known in the art and have been described in such literature as the Physician's Desk Reference (56th ed., 2002 and 57th ed., 2003).

5.5.5.1. Other Therapies

The present invention provides methods of preventing, managing, treating or ameliorating an autoimmune disorder or one or more symptoms thereof, said methods comprising administering to a subject in need thereof a liquid formulation of the invention and one or more therapies (e.g., prophylactic or therapeutic agents) other than antibodies or antibody fragments that immunospecifically bind to integrin αᵥβ₃. Any agent or therapy which is known to be useful, or which has been used or is currently being used for the prevention, management, treatment or amelioration of an autoimmune disorder or one or more symptoms thereof can be used in combination with a liquid formulation of the invention in accordance with the invention described herein. Examples of such agents include, but are not limited to, immunomodulatory agents, anti-inflammatory agents and TNF-α antagonists. Specific examples of immunomodulatory agents, anti-inflammatory agents and TNF-α antagonists which can be used in combination with a liquid formulation of the invention for the prevention, management, treatment or amelioration of an autoimmune disorder are disclosed herein above.

In specific embodiments, patients with multiple sclerosis (MS) are administered a prophylactically or therapeutically effective amount of a liquid formulation of the invention in combination with other agents or therapies useful in prevention, treatment, management and amelioration of MS including but not limited to: IFN-β1b (Betaseron) (e.g., 8.0 million international unites (M11) is administered by subcutaneous injection every other day); IFN-β1a (Avonex) (e.g., 6.0 MIU is administered by intramuscular injection once every week); glatiramer acetate (Copaxone) (e.g., 20 mg is administered by subcutaneous injection every day); mitoxantrone (e.g., 12 mg/m² is administered by intravenous infusion every third month); azathioprine (e.g., 2-3 mg/kg body weight is administered orally each day); methotrexate (e.g., 7.5 mg is administered orally once each week); cyclophosphamide; intravenous immunoglobulin (e.g., 0.15-0.2 g/kg body weight administered monthly for up to 2 years); glucocorticoids; methylprednisolone (e.g., administered in bimonthly cycles at high doses); 2-chlorodeoxyadenosine (cladribine); baclofen (e.g., 15 to 80 mg/d in divided doses, or orally in higher doses up to 240 mg/d, or intrathecally via an indwelling catheter); cyclobenzaprine hydrochloride (e.g., 5-10 mg bid or tid); clonazepam (e.g., 0.5 to 1.0 mg tid, including bedtime dose); clonidine hydrochloride (e.g., 0.1 to 0.2 mg tid, including a bedtime dose); carbamazepine (e.g., 100-1200 mg/d in divided, escalating doses); gabapentin (e.g., 300-3600 mg/d); dilantin (e.g., 300-400 mg/d); amitriptyline (e.g., 25-150 mg/d); baclofen (e.g., 10-80 mg/d); primidone (e.g., 125-250 mg bid or tid); ondansetron (e.g., 4 to 8 mg bid or tid); isoniazid (e.g., up to 1200 mg in divided doses); oxybutynin (e.g., 5 mg bid or tid); tolterodine (e.g., 1-2 mg bid); propantheline (e.g., 7.5 to 15 mg qid); bethanecol (e.g., 10-50 mg tid or qid); terazosin hydrochloride (e.g., 1-5 mg at bedtime); sildenafil citrate (e.g., 50-100 mg po prn); amantading (e.g., 100 mg bid); pemoline (e.g., 37.5 mg bid); high dose vitamins; calcium orotate; gancyclovir; antibiotic; and plasma exchange.

In specific embodiments, patients with psoriasis are administered a prophylactically or therapeutically effective amount of a liquid formulation of the invention in combination with other agents or therapies useful in prevention, treatment, management and amelioration of psoriasis including but not limited to: topical steroid cream or ointment; tar (examples including but not limited to, Estar, Psorigel, Fototar cream, and LCD 10% in Nutraderm lotion or mixed directly with triamcinolone 0.1% cream); occlusion; topical vitamin D analogue (a non-limiting example is calcipotriene ointment); ultraviolet light; PUVA (psoralen plus ultraviolet A); methotrexate (e.g., up to 25 mg once weekly or in divided doses every 12 hours for three doses once a week); synthetic retinoid (a non-limiting examples is etretinate, e.g., in dosage of 0.5-1 mg/kg/d); immunomodulatory therapy (a non-limiting example is cyclosporine); sulfasalazine (e.g., in dosages of 1 g three times daily).

In specific embodiments, patients with Crohn's disease are administered a prophylactically or therapeutically effective amount of a liquid formulation of the invention in combination with other agents or therapies useful in prevention, treatment, management and amelioration of Crohn's disease including but not limited to: antidiarrheals (non-limiting examples are loperamide 2-4 mg up to 4 times a day, diphenoxylate with atropine 1 tablet up to 4 times a day, tincture of opium 8-15 drops up to 4 times a day, cholestyramine 2-4 g or colestipol 5 g once or twice daily); antispasmodics (non-limiting examples are propantheline 15 mg, dicyclomine 10-20 mg, or hyoscyamine 0.125 mg given before meals); 5-aminosalicylic acid agents (non-limiting examples are sulfasalazine 1.5-2 g twice daily, mesalamine (Asacol) and its slow release form (Pentasa), especially at high dosages, e.g., Pentasa 1 g four times daily and Asacol 0.8-1.2 g four times daily); corticosteroids; immunomodulatory drugs (non-limiting examples are azathioprine (1-2 mg/kg), mercaptopurine (50-100 mg), cyclosporine, and methotrexate); antibiotics; TNF inhibitors including but not limited to Inflixmab; immunosuppressive agents including but not limited to, tacrolimus, mycophenolate mofetil, and thalidomide; anti-inflammatory cytokines including but not limited to IL-10 and IL-11; nutritional therapies; enteral therapy with elemental diets (e.g., Vivonex for 4 weeks); and total parenteral nutrition.

In specific embodiments, patients with lupus erythematosus are administered a prophylactically or therapeutically effective amount of a liquid formulation of the invention in combination with other agents or therapies useful in prevention, treatment, management and amelioration of lupus erythematosus including but not limited to: antimalarials (including but not limited to, hydroxychloroquine); glucocorticoids (e.g., low dose, high dose, or high-dose intravenous pulse therapy can be used); immunosuppressive agents (including but not limited to, cyclophosphamide, chlorambucil, and azanthioprine); cytotoxic agents (including but not limited to methotrexate and mycophenolate mofetil); androgenic steroids (including but not limited to danazol); and anticoagulants (including but not limited to warfarin).

5.6. Methods of Administering the Antibody Formulations

The invention provides methods of treatment, management, prophylaxis, and amelioration of an inflammatory disorder, an autoimmune disorder, a disorder associated with aberrant expression and/or activity of integrin α_{V}β₃, a disorder associated with abnormal bone metabolism, a disorder associated with aberrant angiogenesis, cancer or one or more symptoms thereof by administrating to a subject of an effective amount of liquid formulations of the invention. Various delivery systems are known and can be used to administer a liquid formulation of the present invention or a prophylactic or therapeutic agent. Methods of administering antibody liquid formulations of the present invention or a therapy (e.g., a prophylactic or therapeutic agent) include, but are not limited to, parenteral administration (e.g., intradermal, intramuscular, intraperitoneal, intravenous and subcutaneous), epidural administration, topical administration, and mucosal administration (e.g., intranasal and oral routes). In a specific embodiment, liquid formulations of the present invention are administered intramuscularly, intravenously, or subcutaneously. In a preferred embodiment, the liquid formulations of the invention are administered subcutaneously. The formulations may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In a specific embodiment, the liquid formulations of the invention are administered intratumorally or at the site of inflammation.

Generally, the antibodies or antibody fragments that immunospecifically bind to integrin α_{V}β₃ contained in the liquid formulations of the invention are derived from a subject that is of the same species origin or species reactivity as recipient of the liquid formulations of the invention. Thus, in a preferred embodiment, liquid formulations of the invention comprising human or humanized antibodies that immunospecifically bind to integrin α_{V}β₃ contained in the liquid formulations of the invention are administered to a human patient for therapy or prophylaxis.

The invention also provides that a liquid formulation of the present invention is packaged in a hermetically sealed container such as an ampoule or sachette indicating the quantity of antibody or antibody fragment. Preferably, the liquid formulations of the present invention are in a hermetically sealed container indicating the quantity and concentration of the antibody or antibody fragment. Preferably, the liquid formulation of the present invention is supplied in a hermetically sealed container and comprises at least 15 mg/ml, 20 mg/ml, 30 mg/ml, 40 mg/ml, 50 mg/ml, 60 mg/ml, 70 mg/ml, 80 mg/ml, 90 mg/ml, 100 mg/ml, 150 mg/ml, 175 mg/ml, 200 mg/ml, 250 mg/ml, or 300 mg/ml of an antibody or fragment thereof that immunospecifically binds to integrin α_{V}β₃, in a quantity of 1 ml, 2 ml, 3 ml, 4 ml, 5 ml, 6 ml, 7 ml, 8 ml, 9 ml, 10 ml, 15 ml, or 20 ml and, most preferably, 1.2 ml. In a specific embodiment of the invention, a liquid formulation of the invention is supplied in a hermetically sealed container and comprises at least 15 mg/ml, at least 20 mg/ml, at least 25 mg/ml, at least 50 mg/ml, at least 100 mg/ml, at least 150 mg/ml, at least 175 mg/ml, at least 200 mg/ml, at least 250 mg/ml or at least 300 mg/ml of an antibody or fragment thereof that immunospecifically binds to integrin α_{V}β₃ (e.g., VITAXIN® or an antigen-binding fragment thereof) for intravenous injections, and at least 15 mg/ml, 20 mg/ml, 50 mg/ml, 80 mg/ml 100 mg/ml, 150 mg/ml, 175 mg/ml, 200 mg/ml, 250 mg/ml or 300 mg/ml an antibody or fragment thereof that immunospecifically binds to integrin α_{V}β₃ (e.g., VITAXIN® or a fragment thereof) for repeated subcutaneous administration.

The amount of a liquid formulation of the present invention which will be effective in the treatment, management, prevention or amelioration of an inflammatory disorder, an autoimmune disorder, a disease associated with aberrant expression and/or activity of integrin α_{V}β₃, a disease or disorder associated with aberrant bone metabolism, a disease or disorder associated with aberrant angiogenesis, cancer or one or more symptoms thereof can be determined by standard clinical techniques well-known in the art or described herein. The precise dose to be employed in the formulation will also depend on the route of administration, and the seriousness of the inflammatory disorder, autoimmune disorder or cancer, and should be decided according to the judgment of the practitioner and each patient's circumstances. Effective doses may be extrapolated from dose-response curves derived from in vitro or animal model test systems.

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 15 mg/kg and 50 mg/kg of the patient's body weight, more preferably 5 to 25 mg/kg or 5 to 15 mg/kg of the patient's body weight. In a specific embodiment, the dosage administered to a patient is 5 mg/kg, 8 mg/kg, 10 mg/kg, 15 mg/kg, 20 mg/kg, 25 mg/kg, 30 mg/kg or 50 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage, volume and frequency of administration of liquid formulations of the present invention may be reduced by increasing the concentration of an antibody or a fragment thereof in the formulations, increasing affinity and/or avidity of the antibody or a fragment thereof, and/or increasing the half-life of the antibody or a fragment thereof.

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In a specific embodiment, 0.1 to 20 mg/kg/week, preferably 1 to 15 mg/kg/week, more preferably 2 to 8 mg/week, even more preferably 3 to 7 mg/kg/week, and most preferably 4 to 6 mg/kg/week of an antibody or fragment thereof that immunospecifically binds to integrin α_{V}β₃ (e.g., VITAXIN® or a fragment thereof) in a liquid formulation of the invention is administered to a subject with an inflammatory disorder, an autoimmune disorder or cancer. In another embodiment, a subject is administered one or more doses of a prophylactically or therapeutically effective amount of a liquid formulation of the invention, wherein the prophylactically or therapeutically effective amount is not the same for each dose.

In one embodiment, a liquid formulation of the invention is administered in a dosing regimen that maintains the plasma concentration of the antibody immunospecific for α_{V}β₃ at a desirable level (e.g., about 0.1 to about 100 μg/ml), which continuously blocks the integrin α_{V}β₃ activity. In a specific embodiment, the plasma concentration of the antibody is maintained at 0.2 μg/ml, 0.5 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 15 μg/ml, 20 μg/ml, 25 μg/ml, 30 μg/ml, 35 μg/ml, 40 μg/ml, 45 μg/ml or 50 μg/ml. The plasma concentration that is desirable in a subject will vary depending on several factors, including but not limited to, the nature of the disease or disorder, the severity of the disease or disorder and the condition of the subject. Such dosing regimens are especially beneficial in prevention, treatment, management and amelioration of a chronic disease or disorder.

In one embodiment, a liquid formulation of the invention is administered to a subject with a disease or disorder that associated with abnormal bone metabolism using a dosing regimen that maintains the plasma concentration of the antibody or antibody fragment that immunospecifically binds to integrin α_{V}β₃ at a level that blocks at least 40%, preferably at least 50%, at least 55%, at least 60%, at least 65%, at least 70%, at least 75%, at least 80%, at least 85%, at least 90% or at least 95% of bone resorption. In a specific embodiment, the plasma concentration of the antibody or antibody fragment that immunospecifically binds to integrin α_{V}β₃ is maintained at about 0.1 μg/ml to about 100 μg/ml in a subject with a disease or disorder that associated with abnormal bone metabolism.

In specific embodiments, a liquid formulation of the invention comprising a conjugated antibody or antibody fragment immunospecific for α_{V}β₃ is administered intermittently. As used herein, “a conjugated antibody or antibody fragment” refers to an antibody or antibody fragment that is conjugated or fused to another moiety, including but not limited to, a heterologous peptide, polypeptide, another antibody or antibody fragment, a marker sequence, a diagnostic agent, a therapeutic moiety, a therapeutic drug, a radioactive metal ion, a polymer, albumin, and a solid support.

In another embodiment, a subject, preferably a human, is administered one or more doses of a prophylactically or therapeutically effective amount of an antibody or antibody fragment that immunospecifically binds to integrin α_{V}β₃ (e.g., VITAXIN® or a fragment thereof) in a liquid formulation of the invention, wherein the dose of a prophylactically or therapeutically effective amount of the antibody or antibody fragment in the liquid formulation of the invention administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

In another embodiment, a subject, preferably a human, is administered one or more doses of a prophylactically or therapeutically effective amount of an antibody or antibody fragment that immunospecifically binds to integrin α_{V}β₃ (e.g., VITAXIN® or a fragment thereof) in a liquid formulation of the invention, wherein the dose of a prophylactically or therapeutically effective amount of the antibody or antibody fragment in the liquid formulation of the invention administered to said subject is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

The dosages of prophylactic or therapeutically agents are described in the Physicians' Desk Reference (56th ed., 2002 and 57th ed., 2003).

5.7. Biological Assays

Several aspects of the liquid formulations of the invention are preferably tested in vitro, in a cell culture system, and in an animal model organism, such as a rodent animal model system, for the desired therapeutic activity prior to use in humans. For example, assays which can be used to determine whether administration of a specific liquid formulation of the invention is indicated, include cell culture assays in which a patient tissue sample is grown in culture, and exposed to or otherwise contacted with a liquid formulation of the invention, and the effect of such composition upon the tissue sample is observed. The tissue sample can be obtained by biopsy from the patient. This test allows the identification of the therapeutically most effective prophylactic or therapeutic molecule(s) for each individual patient. In various specific embodiments, in vitro assays can be carried out with representative cells of cell types involved in an autoimmune disorder, an inflammatory disorder, a disorder associated with aberrant expression and/or activity of integrin αᵥβ₃, a disorder associated with abnormal bone metabolism, a disorder associated with aberrant angiogenesis, or cancer (e.g., endothelial cells, activated T cells, osteoclasts and B cells), to determine if a liquid formulation of the invention has a desired effect upon such cell types. A lower level of proliferation or survival of the contacted cells indicates that the liquid formulation of the invention is effective to treat the condition in the patient. Alternatively, instead of culturing cells from a patient, a liquid formulation of the invention may be screened using cells of a tumor or malignant cell line or an endothelial cell line. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos and myc) or cell cycle markers; cell viability can be assessed by trypan blue staining, differentiation can be assessed visually based on changes in morphology, etc.

The binding specificity, affinity and functional activity of the antibody or antibody fragment in the liquid formulations of the invention can be characterized in various in vitro binding and cell adhesion assays known in the art, including but limited to, those that are disclosed in International Publication Nos. WO 00/78815 and WO 02/070007, U.S. Pat. No. 6,248,326, U.S. Pat. No. 6,472,403, Pecheur et al., The FASEB J. 16(10): 1266-8 (2002), Ahmed et al., The Journal of Histochemistry & Cytochemistry 50:1371-1379 (2002), all of which are incorporated herein by reference.

The binding specificity of the antibody or antibody fragment in the liquid formulations of the invention can be assessed by measuring binding to αᵥβ₃ and its crossreactivity to other αᵥ- or β₃-containing integrins. Specifically, binding specificity can be assessed by measuring binding to α_{11b}β₃, the major integrin expressed on platelets, and to αᵥβ₅, an integrin found prevalent on endothelial cells and connective tissue cell types. Briefly, to determine crossreactivity, integrins are coated onto an ELISA plate and a series of antibody dilutions are measured for antibody binding activity against αᵥβ₃ and the other integrins. The integrins αᵥβ₃ and αᵥβ₅ can be isolated by know techniques in the art, e.g., by affinity chromatography as described in Cheresh, Proc. Natl. Acad. Sci. USA 84:6471-6475 (1987), and Cheresh and Spiro, J. Biol. Chem. 262:17703-17711 (1987). In a specific embodiment, an anti-αᵥβ₃ antibody affinity column is used to isolate αᵥβ₃ from an octylglucoside human placental lysate, whereas an anti-αᵥ affinity column is used to isolate αᵥβ₅ from the αᵥβ₃-depleted column flow through. Antibody binding activity is assessed by ELISA using a goat anti-human IgG-alkaline phosphatase conjugate. A purified human IgG₁ antibody can be used as a control.

In another embodiment, the binding affinity and specificity are assessed in a competitive binding assay with the parental anti-integrin αᵥβ₃ antibody or antibody fragment against integrin αᵥβ₃. Competitive binding is measured in an ELISA assay. Binding of the antibody is determined in the presence of increasing concentrations of antibody competitor. Alternatively, the control competitor antibody is again a human IgG₁.

In another embodiment, binding affinity and specificity are assessed by measuring the inhibitory activity of the antibody or antibody fragment on integrin αᵥβ₃ binding to fibrinogen. Briefly, integrin αᵥβ₃ is plated onto ELISA plates. Inhibitory activity of the antibody or antibody fragment is determined by measuring the amount of bound biotinylated fibrinogen in the presence of increasing concentrations of antibody or control antibody. Streptavidin alkaline phosphatase is used to detect the bound fibrinogen.

In another embodiment, the specificity of the antibody or antibody fragment binding is assessed by the inhibition of integrin αᵥβ₃ binding in cell adhesion assays. Endothelial cell adhesion events are an important component in the angiogenic process and inhibition of αᵥβ₃ is known to reduce the neovascularization of tumors and thereby reduce the rate of tumor growth. The inhibition of αᵥβ₃-mediated cell attachment by anti-integrin αᵥβ₃ antibody in these assays is indicative of the inhibitory activity expected when this antibody is used in situ or in vivo. Briefly, integrin αᵥβ₃-positive M21 melanoma cells grown in RPMI containing 10% FBS are used for these cell binding assays. Cells are released from the culture dish by trypsinization and re-suspended in adhesion buffer at a concentration of 4×10⁵ cells/ml. The antibody and the control antibody are diluted to the desired concentration in 250 μl adhesion buffer (10 mM Hepes, 2 mM MgCl₂, 2 mM CaCl₂, 0.2 mM MnCl₂, and 1% BSA in Hepes buffered saline at pH 7.4) and added to wells of a 48-well plate precoated with fibrinogen. Each well is coated with 200 μl fibrinogen at a concentration of 10 μg/ml for 1 hour at 37° C. For the assay, an equal volume of cells (250 μl) containing the antibody or isotype matched control antibody is added to each of the wells, mixed by gentle shaking and incubated for 20 minutes at 37° C. Unbound cells are removed by washing with adhesion buffer until no cells remained in control wells coated with BSA alone. Bound cells are visualized by staining with crystal violet which is subsequently extracted with 100 μl acetic acid (10%) and quantitated by determining the absorbance of the solubilized dye at 560 nm.

In another embodiment, the inhibitory activity of an antibody or antibody fragment that immunospecifically binds to integrin αᵥβ₃ is also tested in an endothelial cell migration assay. In this regard, the Transwell cell migration assay is used to assess the ability of Vitaxin to inhibit endothelial cell migration (Choi et al., J. Vascular Surg., 19:125-134 (1994) and Leavesly et al., J. Cell Biol., 121:163-170 (1993). Briefly, human umbilical vein endothelial cells in log phase and at low passage number are harvested by gentle trypsinization, wash and resuspend at a concentration of 2×10⁶ cells/ml in 37° C. HBS containing 1% BSA (20 mM Hepes, 150 mM NaCl, 1.8 mM MgCl₂, 1.8 mM CaCl₂, 5 mM KCl, and 5 mM glucose, pH 7.4). Antibodies are diluted to 10 μl/ml from stock solutions. Antibodies are added to cells in a 1:1 dilution (final concentration of antibodies=5 μg/ml; final concentration of cells=1×10⁶ cells/ml) and incubated on ice for 10-30 minutes. The cell/antibody suspensions (200 μl to each compartment) are then added to the upper compartments of a Transwell cell culture chamber, the lower compartments of which had been coated with 0.5 ml of 10 μg/ml vitronectin (in HBS). Vitronectin serves as the chemoattractant for the endothelial cells. The chambers are placed at 37° C. for 4 hours to allow cell migrate to occur. Visualization of cell migration is performed by first removing the remaining cells in the upper compartment with a cotton swab. Cells that had migrated to the lower side of insert are stained with crystal violet for 30 minutes, followed by solubilization in acetic acid and the absorbance of the dye is measure at a wavelength of 550 nm. The amount of absorbance is directly proportional to the number of cells that have migrated from the upper to the lower chamber.

In a preferred embodiment, BIAcore kinetic analysis is used to determine the binding on and off rates of antibodies or antibody fragments to integrin αᵥβ₃. BIAcore kinetic analysis comprises analyzing the binding and dissociation of integrin αᵥβ₃ from chips with immobilized antibodies or fragments thereof on their surface.

Additional examples of in vitro assays, e.g., Western blotting analysis, flow cytometric analysis, cell adhesion assay to cortical bone and extracellular matrix proteins, cell migration assay, cell invasion assay, and cell proliferation assay, can be found in Pecheur et al., The FASEB J. 16(10): 1266-8 (2002), of which the entire text is incorporated herein by reference.

The liquid formulations of the invention can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, the liquid formulations of the invention are tested in a mouse model system. Such model systems are widely used and well-known to the skilled artisan such as the SCID mouse model or transgenic mice where a mouse integrin α_{V}β₃ is replaced with the human integrin α_{V}β₃, nude mice with human xenografts, animal models wherein an antibody or fragment thereof that immunospecifically binds to integrin α_{V}β₃ recognizes the same target as VITAXIN,® such as hamsters, rabbits, etc. known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties. The liquid formulations of the invention can be administered repeatedly. Several aspects of the procedure may vary.

Various animal models known in the art that are relevant to a targeted disease or disorder, e.g., inflammatory diseases, autoimmune diseases, diseases or disorders associated with aberrant bone metabolism and/or aberrant angiogenesis, or cancer can be used, including but not limited to, those that are disclosed in International Publication Nos. WO 00/78815, U.S. Pat. No. 6,248,326, U.S. Pat. No. 6,472,403, Pecheur et al., The FASEB J. 16(10): 1266-8 (2002), Ahmed et al., The Journal of Histochemistry & Cytochemistry 50:1371-1379 (2002), all of which are incorporated herein by reference.

In one embodiment, inhibition of tumor growth by a liquid formulation of the invention is tested in two animal models. The first model measures angiogenesis in the chick chorioallantoic membrane (CAM). This assay is a well recognized model for in vivo angiogenesis because the neovascularization of whole tissue is occurring. Specifically, the assay measures growth factor induced angiogenesis of chicken CAM vessels growing toward the growth factor-impregnated filter disk or into the tissue grown on the CAM. Inhibition of neovascularization is based on the amount and extent of new vessel growth or on the growth inhibition of tissue on the CAM. The assay has been described in detail by others and has been used to measure neovascularization as well as the neovascularization of tumor tissue (Ausprunk et al., Am. J. Pathol., 79:597-618 (1975); Ossonski et al., Cancer Res., 40:2300-2309 (1980); Brooks et al., Science, 264:569-571 (1994a) and Brooks et al., Cell, 79:1157-1164 (1994b). Briefly, for growth factor induced angiogenesis filter disks are punched from #1 Whatman Qualitative Circles using a skin biopsy punch. Disks are first sterilized by exposure to UV light and then saturated with varying concentrations of TNF-α of HBSS as a negative control (for at least 1 hour) under sterile conditions. Angiogenesis is induced by placing the saturated filter disks on the CAMs. Inhibition of angiogenesis is performed by treating the embryos with various amounts of Vitaxin and controls (antibody or purified human IgG₁). The treatments are performed by intravenous injection approximately 24 hours after disk placement. After 48 hours, CAMs are dissected and angiogenesis is scored on a scale of 1-4. HBSS saturated filter disks are used as the negative control, representing angiogenesis that may occur in response to tissue injury in preparing CAMs, and, values for these CAMs are subtracted out as background. Purified human IgG₁ is used as the negative control for injections since Vitaxin is of the human IgG₁ subclass.

In addition to the above described CAM assay using growth factor-induced neovascularization, additional assays can be performed utilizing tumor-induced neovascularization. For these assays, angiogenesis is induced by transplanting of αᵥβ₃-negative tumor fragments into the CAMs. The use of αᵥβ₃-negative tumor fragments ensures that any inhibition of tumor growth is due to the inhibition of αᵥβ₃-mediated neovascularization by CAM-derived endothelial cells and not to adhesion events mediated by αᵥβ₃ present on the tumor cells. Inhibition of tumor growth is assessed by placing a single cell suspension of FG (8×10⁶ cells, pancreatic carcinoma) and Hep-3 cells (5×10⁵ cells, laryngeal carcinoma) onto CAMs in 30 μl. One week later, tumors are removed and cut into approximately 50 mg fragments at which time they are placed onto new CAMs. After 24 hours of this second placement, embryos are injected intravenously with Vitaxin or human IgG₁ as a negative control. The tumors are allowed to grow for about 7 days following which they are removed and weighed.

In a second animal model, the inhibition of Vx2 carcinoma cells in rabbits is used as a measure of inhibitory effect on tumors of a liquid formulation of the invention. The Vx2 carcinoma is a transplantable carcinoma derived from a Shope virus-induced papilloma. It was first described in 1940 and has since been used extensively in studies on tumor invasion, tumor-host interactions and angiogenesis. The Vx2 carcinoma is fibrotic in nature, highly aggressive, and exhibits features of an anaplastic type carcinoma. Propagation of Vx2 tumor is accomplished through serial transplantation in donor rabbits. Following subcutaneous transplantation, it has been reported that after an initial inflammatory reaction, host repair mechanisms set in between days 2 and 4. This repair mechanism is characterized by the formation of new connective tissue and the production of new capillaries. The newly formed capillaries are restricted to the repair zone at day 4, however, by day 8 they have extended to the outer region of the tumor. These characteristics and the pharmacokinetics of a liquid formulation of the invention in rabbits can be used to determine initial doses and scheduling of treatments for these experiments.

Growth of Vx2 tumors in the above animal model is used to study the effect of a liquid formulation of the invention after early administration on primary tumor growth in rabbits implanted subcutaneously with Vx2 carcinoma. Briefly, Vx2 tumors (50 mg) are transplanted into the inner thigh of rabbits through an incision between the skin and muscle. Measurements of the primary tumor are taken throughout the experiment through day 25.

In another embodiment, BALB/c nu/nu mice are used as animal models to study different diseases, especially those associated with aberrant bone metabolism and/or aberrant angiogenesis. Different cell lines (e.g., CHO, or a type of cancer cells such as breast cancer cells) expressing αᵥβ₃ in various forms can be injected intravenously into the nude mice. See Pecheur et al., supra. For example, CHO cells are transfected with various cDNA constructs of αᵥβ₃ (e.g., wild-type, mutated forms) and injected intravenously into nude mice. The effects of αᵥβ₃ (with various level of activity because of the mutations) and anti-αᵥβ₃ antibodies on bone metastases can be assessed by, e.g., radiograph, histological examination of bone tissue or statistical analysis.

In another embodiment, animals (healthy or previously constructed animal models) in space environment (e.g., space shuttle) can be used to assess the antibodies of the invention. Since astronauts in long space flights have been shown to lose bone density in a way that is similar to osteoporosis patient, but ten times faster than in people who have the advantage of Earth's gravity (see BioWorld Today, 14:13, Jan. 21, 2003), animals in space environment are ideal osteoporosis model for determining the effects of antibodies or antibody fragments of the invention on osteoporosis or other diseases related to aberrant bone metabolism and/or aberrant angiogenesis.

In another embodiment, SCID mice with subcutaneously implanted human bone fragments (SCID-human-bone model) are used as an animal model to assess the effects of the antibodies or antibody fragments of the invention on diseases associated with aberrant bone metabolism and/or aberrant angiogenesis. For examples, cancer cells (e.g., human prostate cancer cells) are injected directly into human bone fragments in the animal model. At the same time, antibody treatment is initiated. The effects of the antibody or antibody fragment of the invention on bone metastases or angiogenesis can be assessed by comparing to a control group. See Nemeth et al., Clinical & Experimental Metastasis, 19 (Supp. 1):47 (2002).

The anti-inflammatory activity of the liquid formulations of the invention can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993). Experimental and spontaneous animal models of inflammatory arthritis and autoimmune rheumatic diseases can also be used to assess the anti-inflammatory activity of the liquid formulation of the invention.

The principle animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993), incorporated herein by reference in its entirety.

The anti-inflammatory activity of the liquid formulations of the invention can be assessed using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al., “Carrageenan-Induced Arthritis in the Rat,” Inflammation, 24(2): 141-155, (2000). Also commonly used are zymosan-induced inflammation animal models as known and described in the art.

The anti-inflammatory activity of the liquid formulations of the invention can also be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for the anti-inflammatory activity of most NSAIDs, and is considered predictive of human efficacy. The anti-inflammatory activity of the liquid formulation of the invention is expressed as the percent inhibition of the increase in hind paw weight of the test group relative to the vehicle dosed control group.

In a specific embodiment of the invention where the experimental animal model used is adjuvant-induced arthritis rat model, body weight can be measured relative to a control group to determine the anti-inflammatory activity of the liquid formulations of the invention. Alternatively, the efficacy of the liquid formulations of the invention can be assessed using assays that determine bone loss. Animal models such as ovariectomy-induced bone resorption mice, rat and rabbit models are known in the art for obtaining dynamic parameters for bone formation. Using methods such as those described by Yositake et al. or Yamamoto et al., bone volume is measured in vivo by microcomputed tomography analysis and bone histomorphometry analysis. Yoshitake et al., “Osteopontin-Deficient Mice Are Resistant to Ovariectomy-Induced Bone Resorption,” Proc. Natl. Acad. Sci. 96:8156-8160, (1999); Yamamoto et al., “The Integrin Ligand Echistatin Prevents Bone Loss in Ovariectomized Mice and Rats,” Endocrinology 139(3):1411-1419, (1998), both incorporated herein by reference in their entirety.

Additionally, animal models for inflammatory bowel disease can also be used to assess the efficacy of the liquid formulations of the invention (Kim et a I., 1992, Scand. J. Gastroentrol. 27:529-537; Strober, 1985, Dig. Dis. Sci. 30(12 Suppl):3S-10S). Ulcerative cholitis and Crohn's disease are human inflammatory bowel diseases that can be induced in animals. Sulfated polysaccharides including, but not limited to amylopectin, carrageen, amylopectin sulfate, and dextran sulfate or chemical irritants including but not limited to trinitrobenzenesulphonic acid (TNBS) and acetic acid can be administered to animals orally to induce inflammatory bowel diseases.

Animal models for asthma can also be used to assess the efficacy of the liquid formulations of the invention. An example of one such model is the murine adoptive transfer model in which aeroallergen provocation of TH1 or TH2 recipient mice results in TH effector cell migration to the airways and is associated with an intense neutrophilic (TH1) and eosinophilic (TH2) lung mucosal inflammatory response (Cohn et al., 1997, J. Exp. Med. 1861737-1747).

Animal models for autoimmune disorders can also be used to assess the efficacy of the liquid formulations of the invention. Animal models for autoimmune disorders such as type 1 diabetes, thyroid autoimmunity, systemic lupus eruthematosus, and glomerulonephritis have been developed (Flanders et al., 1999, Autoimmunity 29:235-246; Krogh et al., 1999, Biochimie 81:511-515; Foster, 1999, Semin. Nephrol. 19:12-24).

Further, any assays known to those skilled in the art can be used to evaluate the prophylactic and/or therapeutic utility of the liquid formulations of the inventions disclosed herein for autoimmune disorders, inflammatory diseases, diseases or disorders associated with aberrant bone metabolism or aberrant angiogenesis, and/or cancers. Assays known in the art (e.g., assays described above) can also be used to evaluate the prophylactic and/or therapeutic utility of the liquid formulations of the invention in combination with one or more other therapies.

The effect of the liquid formulations of the invention on peripheral blood lymphocyte counts can be monitored/assessed using standard techniques known to one of skill in the art. Peripheral blood lymphocytes counts in a subject can be determined by, e.g., obtaining a sample of peripheral blood from said subject, separating the lymphocytes from other components of peripheral blood such as plasma using, e.g., Ficoll-Hypaque (Pharmacia) gradient centrifugation, and counting the lymphocytes using trypan blue. Peripheral blood T-cell counts in subject can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen such as CD3, CD4, and CD8 which is conjugated to FITC or phycoerythrin, and measuring the number of T-cells by FACS.

The toxicity and/or efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The liquid formulations of the invention that exhibit large therapeutic indices are preferred. While liquid formulations of the invention that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

5.8. Use of Liquid Formulations in the Analysis of Integrin α_{V}β₃ Expression

A liquid formulation of the invention may be used to visualize the expression of integrin α_{V}β₃ in cells or cell lines, and in tissue sections and biopsies. In certain embodiments, the analysis of tissue samples and biopsies requires the use of frozen tissues. In preferred embodiments, the tissue samples and biopsies are prepared using standard methods for processing and paraffin embedding of tissue while allowing immunohistochemical staining of integrin α_{V}β₃ in the resulting paraffin embedded tissue. Given the invention, such methods can facilitate the analysis of integrin α_{V}β₃ expression in tissue samples from clinical trials, animal models, and biopsies.

A liquid formulation of the invention may be used to evaluate the metastatic potential of a cancer (e.g., lung cancer, breast cancer, prostate cancer, or ovarian cancer) by determining the expression and/or activity level of integrin α_{V}β₃ in cells or cell lines, and in tissue sections and biopsies.

The liquid formulations of the invention comprising labeled antibodies, fragments, derivatives and analogs thereof, that immunospecifically bind to integrin αᵥβ₃ can be used for diagnostic purposes to detect, diagnose, or monitor various diseases and disorders, including but not limited to, inflammatory diseases, autoimmune diseases, diseases associated with aberrant bone metabolism, diseases associated with aberrant angiogenesis and cancer. Such diagnostic techniques are known in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Other diagnostic techniques that are know in the art include, but are not limited to, those disclosed in International Publication No. WO 01/58483, U.S. Pat. No. 6,248,326, Pecheur et al., The FASEB J. 16(10):1266-8 (2002), Ahmed et al., The Journal of Histochemistry & Cytochemistry 50:1371-1379 (2002), all of which are incorporated herein by reference. In a preferred embodiment, antibodies or antibody fragments which immunospecifically bind to integrin αᵥβ₃ are used for diagnostic purposes to detect, diagnosis, or monitor a disease or disorder. The detection or diagnosis of a disease or disorder can be conducted utilizing an effective amount (i.e., an amount effective to be able to detect the expression of integrin αᵥβ₃) of a liquid formulation of the invention in an in vitro and/or in vivo assay using techniques well-known to one of skilled in the art. In a preferred embodiment, a disease or disorder is detected in the subject, preferably a mammalian subject and most preferably a human subject utilizing an effective amount of a liquid formulation of the invention in a standard imaging technique known to one of skilled in the art.

The invention provides methods of detecting or diagnosing a disease or disorder, said methods comprising: a) administering to a subject an effective amount of a liquid formulation of the invention comprising a labeled antibody or antibody fragment that immunospecifically binds to integrin αᵥβ₃; b) waiting for a time interval following the administering for permitting the labeled antibody or antibody fragment to preferentially concentrate or localize at any desired site, e.g., cancerous site, in the subject (and for unbound labeled antibody or antibody fragment to be cleared to background level); c) determining background level; and d) detecting the labeled molecule in the subject, such that detection of labeled antibody or antibody fragment above the background level indicates the presence of the disease.

The invention provides methods of detecting or diagnosing a disease or disorder, said methods comprising: a) administering to a subject an effective amount of a liquid formulation of the invention comprising an antibody or antibody fragment that immunospecifically binds to integrin αᵥβ₃; b) administering to said subject a second labeled antibody or antibody fragment that recognizing the antibody or antibody fragment of the liquid formulation of the invention; c) waiting for a time interval following the administering for permitting the labeled antibody or antibody fragment to preferentially concentrate or localize at any desired site, e.g., cancerous site, in the subject (and for unbound labeled antibody or antibody fragment to be cleared to background level); d) determining background level; and e) detecting the labeled antibody or antibody fragment in the subject, such that detection of labeled antibody or antibody fragment above the background level indicates the presence of the disease.

The tissue analyzed in accordance with methods of the invention in some embodiments are tissues from cancer patients obtained during surgery. See Ahmed et al., The Journal of Histochemistry & Cytochemistry 50:1371-1379 (2002). For example, the tissues from patient with ovary cancer presented for surgery are divided and frozen in cylinders of frozen section embedding medium (OCT) by immersion in isopentane cooled in dry ice. Frozen sections of the tissue are cut at 5 μm thickness and, if not used immediately stored at −20° C. For staining, sections are fixed in cold acetone for 15 minutes and held in Tris buffer (100 mM, pH 7.6). Endogenous peroxidase activity is removed using 3% H₂O₂ in methanol and endogenous biotin activity is blocked using a sequence of diluted egg white (5% in distilled water) and skim milk powder (5% in distilled water), all for 10 minutes. The sections are incubated for 1 hour with αᵥβ₃ Mab in Tris buffer (100 mM, pH 7.6). Antibody binding is amplified using biotin and streptavidin HRP for 15 minutes each and the complex is visualized using diaminobenzidine (DAB). Nuclei are lightly stained with Mayer's hematoxylin and the sections mounted and cover-slipped. An isotype IgG1, suitably diluted, is substituted for the antibody as a negative control. Sections are assessed microscopically for positive DAB staining by trained pathologists, and the degree of staining of αᵥβ₃ expression is scored in a blind fashion.

The invention provides methods for the diagnosis or detection of a disease or disorder in a subject, said methods comprising imaging said subject at a time interval after administering to said subject an effective amount of a liquid formulation of the invention comprising a labeled antibody or antibody fragment that immunospecifically binds to integrin αᵥβ₃, said time interval being sufficient to permit the labeled antibody or antibody fragment to preferentially concentrate at a specific site, e.g., a cancerous site, in said subject, wherein detection of the labeled antibody or antibody fragment localized at the site in the subject indicates the presence of the disease or disorder.

In some embodiments, monitoring of a disease or disorder is carried out by repeating the method for diagnosing the disease or disorder, for example, one month after initial diagnosis, six month after initial diagnosis, and one year after initial diagnosis. In specific embodiments of the invention, the density of a tumor facilitates the detection of said tumor using anti-integrin αᵥβ₃ antibodies in accordance with the method of the invention.

Presence of labeled antibody or antibody fragment can be detected in the patient using methods known in the art for in vivo scanning. These methods depend upon the type of label used. Skilled artisans will be able to determine the appropriate method for detecting a particular label. Methods and devices that may be used in the diagnostic methods of the invention include but are not limited to: computed tomography (CT), whole body scan such as position emission tomography (PET), magnetic resonance imaging (MRI), and sonography. In a specific embodiment, the antibody or antibody fragment is labeled with a radioisotope and is detected in the patient using a radiation responsive surgical instrument (Thurston et al., U.S. Pat. No. 5,441,050). In another embodiment, the antibody or antibody fragment is labeled with a fluorescent compound and is detected in the patient using a fluorescence responsive scanning instrument. In another embodiment, the antibody or antibody fragment is labeled with a positron emitting metal and is detected in the patient using positron emission tomography. In yet another embodiment, the antibody or antibody fragment is labeled with a paramagnetic label and is detected in a patient using magnetic resonance imaging (MRI).

5.9. Kits

The invention provides a pharmaceutical pack or kit comprising one or more containers filled with a liquid formulation of the invention. In a specific embodiment, the liquid formulations of the invention comprise antibodies or antibody fragments recombinantly fused or chemically conjugated to another moiety, including but not limited to, a heterologous protein, a heterologous polypeptide, a heterologous peptide, a large molecule, a small molecule, a marker sequence, a diagnostic or detectable agent, a therapeutic moiety, a drug moiety, a radioactive metal ion, a second antibody, and a solid support. The invention also provides a pharmaceutical pack or kit comprising in one or more first containers a liquid formulation of the invention and in one or more second containers one or more other prophylactic or therapeutic agents useful for the prevention, management or treatment of an inflammatory disorder, a disorder associated with bone metabolism, a disorder associated with aberrant angiogenesis, a disorder associated with aberrant expression and/or activity of integrin αᵥβ₃, an autoimmune disorder or a cancer. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises a liquid formulation of the invention, in one or more containers. In another embodiment, a kit comprises a liquid formulation of the invention, in one or more containers, and one or more other prophylactic or therapeutic agents useful for the prevention, management or treatment of an inflammatory disorder, a disorder associated with bone metabolism, a disorder associated with aberrant angiogenesis, a disorder associated with aberrant expression and/or activity of integrin αᵥβ₃, an autoimmune disorder or a cancer, in one or more other containers. In a specific embodiment, the antibody or antibody fragment included in said liquid formulations is VITAXIN® or an antigen-binding fragment. In an alternative embodiment, the antibody or antibody fragment included in said liquid formulations is not VITAXIN® or an antigen-binding fragment thereof. Preferably, the kit further comprises instructions for preventing, treating, managing or ameliorating a disorder (e.g., using the liquid formulations of the invention alone or in combination with another prophylactic or therapeutic agent), as well as side effects and dosage information for method of administration.

### EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

1.-55. (canceled)

56. An antibody formulation comprising in an aqueous carrier, histidine, polysorbate, and 50 mg/ml or higher of an antibody or antibody fragment that immunospecifically binds to integrin αvβ3, wherein there is no need to reconstitute the formulation prior to administration and wherein said formulation does not include glycine.

57. The formulation of claim 56, wherein said polysorbate is at a concentration of 0.005%.

58. The formulation of claim 56, wherein said antibody formulation is substantially free of inorganic salt.

59. The formulation of claim 56, wherein said histidine is at concentration in the range from about 5 to about 25 mM.

60. The formulation of claim 56, wherein the formulation has a pH in the range between 5.0 and 7.0.

61. The formulation of claim 56, wherein said antibody or antibody fragment has one or more complementary determining regions (CDRs) of LM609.

62. The formulation of claim 56, wherein said antibody or antibody fragment has one or more CDRs of VITAXIN®.

63. The formulation of claim 56, wherein said antibody or antibody fragment has an affinity constant (Ka) of at least 10⁵ M⁻¹.

64. The formulation of claim 56, wherein said antibody or antibody fragment has a dissociation constant (Kd) of less than 5×10⁻⁷ M.

65. The formulation of claim 56, wherein said antibody or antibody fragment comprise a VH CDR1 having an amino acid sequence of SEQ ID NO:1.

66. The formulation of claim 65, wherein said antibody or antibody fragment further comprise a VH CDR2 having an amino acid sequence of SEQ ID NO:2.

67. The formulation of claim 66, wherein said antibody or antibody fragment further comprises a VL CDR2 having an amino acid sequence of SEQ ID NO:5.

68. The formulation of claim 56, wherein the antibody or antibody fragment is stable during storage at 40° C. for at least 15 days as determined by high performance size exclusion chromatography (HPSEC).

69. The formulation of claim 56, wherein the antibody or antibody fragment is stable during storage at about ambient temperature for at least 6 months as determined by HPSEC.

70. The formulation of claim 56, wherein the antibody or antibody fragment is stable during storage at 4° C. for at least 1.5 years as determined by HPSEC.

71. The formulation of claim 68, wherein less than 5% of the antibody or antibody fragment forms an aggregate during the storage as measured by HPSEC.

72. The formulation of claim 69, wherein less than 5% of the antibody or antibody fragment forms an aggregate during the storage as measured by HPSEC.

73. The formulation of claim 70, wherein less than 5% of the antibody or antibody fragment forms an aggregate during the storage as measured by HPSEC.

74. The formulation of claim 68, wherein the antibody or the fragment thereof retains at least 80% of binding ability to integrin αvβ3 compared to a reference antibody representing the antibody prior to the storage.

75. The formulation of claim 69, wherein the antibody or the fragment thereof retains at least 80% of binding ability to integrin αvβ3 compared to a reference antibody representing the antibody prior to the storage.

76. The formulation of claim 70, wherein the antibody or the fragment thereof retains at least 80% of binding ability to integrin αvβ3 compared to a reference antibody representing the antibody prior to the storage.
